(19) United States
(12) Patent Application Publication
(10) Pub. No.: US 2023/0003721 A1
(43) Pub. Date: Jan. 5, 2023

(54) MITOTHERAPEUTICS FOR THE TREATMENT OF BRAIN DISORDERS

(71) Applicants: Ronald L. DAVIS, Palm Beach, FL (US); Ze LIU, Jupiter, FL (US); Boglarka H. VARKUTI, Balatonalmadi (HU); Miklos KEPIRO, Siklos (HU); Courtney M. MACMULLEN, Palm City, FL (US); The University of Florida Research Foundation, Inc., Gainesville, FL (US)

(72) Inventors: Ronald L. DAVIS, West Palm Beach, FL (US); Ze LIU, Jupiter, FL (US); Boglarka H. VARKUTI, Balatonalmadi (HU); Miklos KEPIRO, Siklos (HU); Courtney M. MACMULLEN, Palm City, FL (US)

(21) Appl. No.: 17/756,842

(22) PCT Filed: Nov. 24, 2020
(86) PCT No.: PCT/US2020/062052

§ 371 Date: Jun. 3, 2022

Related U.S. Application Data
(60) Provisional application No. 62/943,005, filed on Dec. 3, 2019.
(60) Provisional application No. 62/977,931, filed on Feb. 18, 2020.

(51) Int. Cl. G01N 33/50; A61P 35/00; A61K 31/353; A61K 31/138; A61K 31/451; A61K 31/403; A61K 31/454; A61K 31/245; A61K 31/12; A61K 31/475; A61K 31/137; A61K 31/495

(52) U.S. Cl. CPC G01N 33/5058; G01N 33/5079; A61P 35/00; A61K 31/353; A61K 31/138; A61K 31/451; A61K 31/403; A61K 31/454; A61K 31/245; A61K 31/12; A61K 31/475; A61K 31/137; A61K 31/495

(57) ABSTRACT

Described herein is a multiplexed and high content screening assay using primary neurons for identifying small molecule modulators of neuronal mitochondrial mitostasis (MnMs). Also described is a high throughput screening assay using primary neurons for identifying small molecules that increase mitochondrial function, identified by measuring the electrochemical potential across the inner mitochondrial membrane and ATP generation. Most MnMs that increased mitochondrial content, length and/or health also increased mitochondrial function without altering neurite outgrowth. Some MnMs protect mitochondria in primary neurons from Aβ(1-42) toxicity, glutamate toxicity, increased oxidative stress and the toxic cellular environment associated with Alzheimer's disease. Some MnMs target mitochondria directly. An MnM also increases the synaptic activity of hippocampal neurons and is potent in vivo, increasing the respiration rate of brain mitochondria after administering the compound to mice. The MnMs were demonstrated to protect the mitochondrial population in neurons in an in vivo model of Alzheimer's Disease. Also described is a method for treating a patient suffering from a disorder characterized by dysfunction of neuronal mitostasis, comprising administering to the patient a therapeutically effective amount of a compound (MnM), or a pharmaceutically acceptable salt thereof.

### RELATED APPLICATIONS

This application claims the benefit of priority to U.S. Provisional Patent Application No. 62/943,005 filed on Dec. 3, 2019, and U.S. Provisional Patent Application No. 62/977,931 filed on Feb. 18, 2020, which applications are incorporated as if fully set forth herein.

### STATEMENT OF GOVERNMENT SUPPORT

This invention was made with government support under grants No. 5R01MH109957 and No. 3R01MH10997-S1 awarded by the National Institutes of Health. The U.S. government has certain rights in the invention.

### BACKGROUND

The unique architecture of post-mitotic neurons with their elaborate dendritic trees and far-reaching axons imposes extraordinary demands on the mitochondrial system for satisfying the neuron's need for energy, calcium buffering, neurotransmitter metabolism, and other physiological processes [1]. These demands are largely met by neuronal mitostasis, which includes the combined processes that maintain mitochondrial number and quality in the various compartments of the neuron across its lifetime. These processes include the regulation of the cell's content of mitochondria through a balance between biogenesis and turnover, the length of each mitochondrion from a balance between fusion and fission, and other processes such as transport across the length of axons and dendrites. Biogenesis is largely controlled by the peroxisome proliferator-activated receptor gamma coactivator 1-alpha (PGC1α), which interacts with multiple transcription factors and nuclear hormone receptors to regulate ˜1500 genes in the nucleus that code for mitochondrial proteins and other factors that coordinate mitochondrial biogenesis and oxidative phosphorylation [2]. The dynamic processes of fission, mediated by dynamin related protein 1 (DRP1), and fusion, mediated by mitofusins and other proteins, provide for quality control over the system. Mitochondria can mix their contents upon fusion, spreading metabolites, enzymes, and other constituents throughout the fused mitochondrion. Damaged parts of a mitochondrion can be segregated into a daughter mitochondrion destined for destruction through fission. However, excessive fission leads to mitochondrial fragmentation and cell death. Increases in mitochondrial length, or elongation, caused by enhanced fusion or decreased fission can be beneficial in some situations, increasing the efficiency of the mitochondrial system and offering protection during stressed conditions [3, 4]. Importantly, dysfunctions of neuronal mitostasis are found in most neuropsychiatric disorders including Alzheimer's disease, Parkinson's disease, amyotrophic lateral sclerosis, mood disorders, schizophrenia and others [5-10]. Mitochondrial failure leads to increased oxidative stress and damage to lipids, proteins and nucleic acids from the generated reactive oxygen species [11]. Toxic Aβ species are reported to accumulate intraneuronally, and within mitochondria, during the development of Alzheimer's disease, impairing energy metabolism, increasing ROS and decreasing ATP production [12].

Surprisingly, very little effort has been made in identifying new compounds that modulate neuronal mitostasis. A few small molecule screens for enhanced mitochondrial function or dynamics have been performed using yeast, human umbilical vein endothelial cells, or other mammalian cell lines [13-15].

Brain and other CNS disorders, and particularly neurodegenerative disorders such as Alzheimer's disease (AD), motor neuron disease or amyotrophic lateral sclerosis (ALS), and Parkinson's disease (PD), cause the failure of many different cellular and molecular systems utilized by the brain for normal function. This has made the development of therapeutics particularly challenging. One of the major systems that fails in nearly all brain disorders is the mitochondrial system in neurons. This system is incredibly important for the function and health of neurons for many different reasons, not the least of which is as a source of energy through the mitochondrial generation of ATP.

Neurodegenerative disorders, for instance, cause three fundamental impairments in the mitochondrial system: (1) There exists increased fragmentation of mitochondria in the disease state due to an imbalance between mitochondrial fission and fusion events. The 6,000-10,000 mitochondria that exist in neurons and supply energy at diverse locations in the cell are in constant flux with each often dividing into two mitochondria. This is balanced by fusion events between mitochondria. These fission:fusion events are placed under the rubric of “mitochondrial dynamics.” (2) Mitochondria produce their energy through the function of the electron transport chain. The function of the electron transport chain is reduced in neurodegenerative disorders leading to reduced synthesis of ATP. (3) The number of mitochondria per neuron is reduced. This may be due to a defect in the biogenesis of mitochondria in the disease and/or due to increased turnover. Neuronal mitochondrial are in a constant state of flux with new mitochondria being born and others that become defective over time being degraded through distinct cellular processes such as mitophagy. These processes, mitochondrial biogenesis and mitophagy are also included in the rubric of mitochondrial dynamics. These observations indicate that neurodegeneration and other brain disorder is associated with dysfunction of the overall mitochondrial system, including both functional aspects of mitochondria and the processes of mitochondrial dynamics.

Although many different systems fail in neurodegeneration, additional systems could fail due to the failure of the mitochondrial system. Thus, dysfunction of the neuronal mitochondrial system can be an upstream failure in the cascade of events leading to eventual neuronal cell death: (1) Mitochondria in neurons are known to become damaged across time due to aging—aging is the primary risk factor for neurodegenerative disorders such as AD; (2) Increased oxidative stress occurs in neurodegenerative disorders—Damaged mitochondria are the major source of reactive oxygen species; (3) Hippocampal neurons are reported to undergo changed in activity levels in neurodegenerative disorders—Mitochondria are reported to establish the set point for activity levels; (4) Chronic neuroinflammation is a major hallmark of neurodegenerative disorders—Oxidized mitochondrial DNA, released from damaged mitochondria, is known to the agonist for the activation of the molecular “inflammasome”; (5) Synapses are lost in neurodegeneration—Mitochondrial function is required for the integrity of synapses and their function, likely through the generation of ATP energy required for synapse function and integrity. Thus, mitochondrial dysfunction ties together many of the major systems failures in AD.

The severe mitochondrial pathology associated with neurodegeneration is recognized and advanced by many investigators as a potential target for the development of therapeutics. Yet, the diversity of the compounds in the pipeline is very limited: most are Type2 diabetes drugs selected to increase brain glucose metabolism. In essence, most therapeutics being developed are unitary in their actions. Notably absent are drugs that have positive effects on the important aspects of mitochondrial dynamics and function that become dysfunction from the insults of neurodegeneration.

### SUMMARY

Drugs that address the deficiencies above can offer broad protection against the three fundamental insults to the mitochondrial system. These are reduced mitochondrial content, fragmentation, and impaired bioenergetic functions, respectively.

The present disclosure satisfies these deficiencies and others by providing, in an embodiment, an in vitro method for determining whether a test agent could be useful as a mitotherapeutic in the treatment of a patient suffering from a neurological or psychiatric disorder. The method comprises:
(a) contacting a test population of brain cells with a mitochondrial reporter for a time sufficient to label mitochondria in live neurons;
(b) incubating the cells with the test agent;
(c) imaging the cells to obtain a visual image of labeled mitochondria;
(d) determining mitochondrial parameters by inspection of the visual image, in comparison to an image of a control population of brain cells not incubated with the test agent, wherein the mitochondrial parameters are selected from:
   concentration of cellular mitochondria;
   mitochondrial length; and
   mitochondrial circularity; and
(e) correlating the presence of one or more results with a conclusion that the test agent is useful as a mitotherapeutic, where the results are selected from:
   an increase in concentration of cellular mitochondria;
   increase in mitochondrial length; and
   decrease in mitochondrial circularity.

In another embodiment, the present disclosure provides an in vitro method for determining whether a test agent is likely toxic to cellular mitochondria. The method comprises:
(a) contacting a test population of cells with a mitochondrial reporter for a time sufficient to label mitochondria in live cells;
(b) incubating the cells with the test agent;
(c) imaging the cells to obtain a visual image of labeled mitochondria;
(d) determining mitochondrial parameters by inspection of the visual image, in comparison to an image of a control population of cells not incubated with the test agent, wherein the mitochondrial parameters are selected from:
   concentration of cellular mitochondria;
   mitochondrial length; and mitochondrial circularity; and
(e) correlating the presence of one or more results with a conclusion that the test agent is likely toxic to mitochondria, wherein the results are selected from:
   a decrease in concentration of mitochondria;
   decrease in mitochondrial length; and
   increase in mitochondrial circularity.

In another embodiment, the present disclosure provides an in vitro method for determining whether a test agent modulates ATP generation from cellular mitochondria. The method comprises:
(a) contacting a test population of cells with a mitochondrial reporter for a time sufficient to label mitochondria in live cells;
(b) incubating the test population of cells with the test agent;
(c) measuring a reporter signal from labeled mitochondria in the test population of cells;
(d) correlating an increase, no change, or decrease in reporter signal from (c), relative to a reporter signal from a control population of cells not incubated with the test agent, to a determination that the test agent enhances, exerts no effect upon, or impairs, respectively, ATP generation from mitochondria in the test population of cells.

In various embodiments, the present disclosure also provides a method for treating a patient suffering from a disorder characterized by dysfunction of neuronal mitostasis or dysfunction of ATP generation. The method comprises administering to the patient a therapeutically effective amount of a compound, or a pharmaceutically acceptable salt thereof, selected from the following table:

cyclizine
alverine citrate
nifedipine
haloperidol
apomorphine hydrochloride
dextromethorphan hydrobromide
orphenadrine citrate
canagliflozin
pargyline hydrochloride
dipyridamole
dyclonine hydrochloride
domperidone
pyrilamine maleate
nefopam
capecitabine
yohimbine hydrochloride
xylazine
budesonide
isotretinon
esomeprazole potassium
tolnaftate
probucol
memantine hydrochloride
lamotrigine
halothane
solifenacin succinate
pyronaridine tetraphosphate
oxelaidin citrate
cloperastine hydrochloride
triclabendazole
clemizole hydrochloride
carbaril
pridinol methanesulfonate
hydroquinidine
pimethixene maleate
genistein
drofenine hydrochloride
clorgiline hydrochloride
exalamide
sulbentine
naftopidil
cholest-5-en-3-one
resveratrol 4′-methyl ether
1r,2s-phenylpropylamine
7-hydroxy-2′-methoxyisoflavone
catechin tetramethylether
2′,4-dihydroxychalcone
2′,4′-dihydroxychalcone
avocatin a
daidzein
10-hydroxycamptothecin
3,4′-dihydroxyflavone
harmine
6-hydroxyflavone
3,5-dihydroxyflavone
1,3-dideacetyl-7-deacetoxy-7-oxokhivorin
phloretin
aleuretic acid
2′,4′-dihydroxychalcone 4′-glucoside
3,7-dihydroxyflavone
dihydrofissinolide
s-isocorydine (+)
levomilnacipran hydrochloride
isocotoin
rhamnetin
euparin
4′-hydroxychalcone

In additional embodiments, the present disclosure also provides a method for treating a patient suffering from a disorder characterized by dysfunction of neuronal mitostasis or dysfunction of ATP generation. The method comprises administering to the patient a therapeutically effective amount of a compound, or a pharmaceutically acceptable salt thereof, selected from the following table:

azelastine hydrochloride
pizotyline malate
doxepin hydrochloride
trimipramine maleate
orphenadrine citrate
nortriptyline hydrochloride
cyclizine
ketotifen fumarate
chlorprothixene hydrochloride
pimethixene maleate
dimenhydrinate
cyclobenzaprine hydrochloride
clemizole hydrochloride
trimeprazine tartrate
promazine hydrochloride
clozapine
thonzylamine hydrochloride
chloropyramine hydrochloride
trihexyphenidyl hydrochloride
procyclidine hydrochloride
pridinol methanesulfonate
drofenine hydrochloride
clidinium bromide
piperidolate hydrochloride
pipenzolate bromide
adiphenine hydrochloride
dyclonine hydrochloride
dibucaine hydrochloride
benoxinate hydrochloride
hycanthone
tolperisone hydrochloride
proparacaine hydrochloride
5alpha-cholestan-3beta-ol-6-one
cyclopamine
androsterone
5,4′-dimethoxy-7-hydroxyisoflavone
phenyl aminosalicylate
genistein
apigenin dimethyl ether
ketanserin tartrate
ritanserin
domperidone
pimozide
carvedilol phosphate
naftopidil
carvedilol
acetophenazine maleate
piperacetazine
thiothixene
dihydrofissinolide
1,7-dideacetoxy-1,7-dioxo-3-deacetylkhivorin
carapin-8(9)-ene
yohimbine hydrochloride
rauwolscine hydrochloride
reserpine
dexpropranolol hydrochloride [R(+)]
propranolol hydrochloride (+/−)
lidocaine hydrochloride
bupivacaine hydrochloride
toremifene citrate
clomiphene citrate
estradiol methyl ether
estrone acetate
celecoxib
diperodon hydrochloride
vinpocetine
nefazodone hydrochloride
butacaine sulfate
nebivolol hydrochloride
lobeline hydrochloride
indole-3-carbinol
trimebutine maleate
tepoxalin
meprylcaine hydrochloride
nimodipine
penfluridol
bisphenol a
nafronyl oxalate
doxazosin mesylate
benzonatate
tigecycline
ajmaline
pyrimethamine
exalamide
hydroxyzine pamoate
mefloquine
tiletamine hydrochloride
ambroxol hydrochloride
colistin sulfate
heteropeucenin, methyl ether
propafenone hydrochloride
quinine ethyl carbonate
aripiprazole
fulvestrant
bussein
canagliflozin
alverine citrate
doxorubicin
sclareol
imidazol-4-ylacetic acid sodium salt
oxiconazole nitrate
naftifine hydrochloride
sertraline hydrochloride
1-hydroxy-3,6,7-trimethoxy-2,8-diprenylxanthone
dehydroabietamide
eugenol
penbutolol sulfate
paroxetine hydrochloride
butyl paraben
triclabendazole
clemastine fumarate
levomilnacipran hydrochloride
sodium nitroprusside
medroxyprogesterone acetate
estradiol cypionate
avanafil
terconazole
oxelaidin citrate
estriol benzyl ether
larixol acetate
amitriptyline hydrochloride
imipramine hydrochloride
benazepril hydrochloride
fluoxetine hydrochloride
orlistat
bifonazole
felodipine
ancitabine hydrochloride
phytol
4′-methoxychalcone
desoxycorticosterone acetate
sparteine sulfate
chlorpromazine
nicardipine hydrochloride
pramoxine hydrochloride

### DESCRIPTION OF THE DRAWINGS

FIG. 1. Assaying Mitochondrial Morphology in Primary Neurons. (A) Mt mice, work flow and timeline of the screen. Mitochondria were visualized in primary mouse neurons by the Cre-dependent expression of mitochondria-targeted GFP (Mt-GFP). The image shows the transduced neurons in the field expressing GFP in somatic and neuritic mitochondria (maximum-projection image of confocal Z-stacks, 3 slices, z=0.7 μm with a 60× objective). (B) Classification of axonal and dendritic mitochondria. Representative images of a neuron (left panel) expressing Cyto-tdTomato and Mt-GFP and zoomed-in segments of a dendrite and two axons. The frequency distributions of mitochondrial length, located either in dendrites or axons, are plotted in the right panel. Best fit lognormal distributions (black lines) show that 15% of the mitochondrial lengths can be found in both axons and dendrites (overlap). nₐₓₒₙₐₗ=517, n_{dendritic}=525. Scalebars=25 μm (left image) and 5 μm (middle and right images). (C) Image analysis. Maximum z-projection of the green channel (Mt-GFP) containing mitochondria and widefield image of neurites (Cyto-tdTomato). After image preprocessing (somatic mitochondria removal from both channels, background subtraction and median filtering of mitochondria, tubeness filtering of neurites), axonal and dendritic mitochondria and neurites were segmented (axonal: 0.5-1.4 μm, dendritic: >2.4 inn) by top-hat filtering followed by adaptive thresholding.

FIG. 2. Hit Parameters for the Mitochondrial Dynamics Screen (A) Representative images showing the effects of FCCP on mitochondrial morphology. 12.5 μM FCCP for 24 h induced fragmentation of dendritic mitochondria compared to DMSO treatment. Scalebar=20 μm. B-E) Mitochondrial morphology of DMSO- and FCCP-treated neurons. Frequency distribution of the area occupied by individual dendritic (B) and axonal (C) mitochondria. FCCP treatment reduced the average area of dendritic mitochondria (mean_{DMSO}=1.44 μm², mean_{FCCP}=1.06 μm²) and decreased the number of dendritic mitochondria (n_{DMSO}=28718, n_{FCCP}=3713). FCCP increased the number of axonal mitochondria (n_{DMSO}=33457, n_{FCCP}=51767) while it did not change the average area of axonal mitochondria (mean_{DMSO}=0.39 mean_{FCCP}=0.39 μm²). Average well CA differed significantly between DMSO and FCCP treatment for dendritic (mean_{DMSO,Dend CA}=1292±213, mean_{FCCP,Dend CA}=123±44) (B, right panel) and axonal mitochondria (mean_{DMSO,Ax CA}=404±79, mean_{FCCP,Ax CA}=629±83 (C, right panel). FCCP treatment produced a significant decrease in dendritic length (D), with average median_{FCCP}=2.77±0.07 μm compared to median_{DMSO}=3.35±0.08 inn. (D, right panel). FCCP treatment also produced a significant increase in the median axonal mitochondrial circularity compared to DMSO treatment (average median_{DMSO}=0.83±0.007 and median_{FCCP}=0.94±0.004, E, right panel), also illustrated by their frequency distribution (E, left panel). (Bar graphs represent mean±s.d., n_{wells}=8/treatment. Unpaired t-tests, ****p<0.0001, n_{DMSO,axonal}=33457 and n_{FCCP, axonal}=51767, n_{DMSO, dendritic}=28718 and n_{FCCP, dendritic}=3713, 32 fields, 8 wells.

FIG. 3. Small Molecule Modulators Increasing Mitochondrial Elongation, Content and Health. (A-C) Primary screen. Average robust Z-scores (N=4 replicate plates) (left panels) and the cumulative distributions (right panels) for median dendritic mitochondrial length (A), dendritic mitochondrial CA (B) and axonal mitochondrial circularity (C). Populations followed a normal distribution (R²_{Elongation}=0.986, R²_{MT Content}=0.987, R²_{Health}=0.987, cumulative gaussian fits—blue lines, right panels). Hits: robust Z-scores >2.5 (A, B) or <2.5 (C). Toxic: median axonal circularity >3 robust Z-scores and median dendritic length <−3 robust Z-scores. D-F) Rescreen of primary hits. Relative frequency distributions of average robust Z-scores (N=10 replicate plates) for median dendritic mitochondrial length (D), dendritic mitochondrial CA (E) and axonal mitochondrial circularity (F). Gaussian fits to frequency histograms of compound (blue, n=149 compounds) and DMSO treated (grey, n=42 wells) neurons yielded population means: M_{DMSO}=−0.05 and M_{Compounds}=1.07 for median dendritic mitochondrial length (D), M_{DMSO}=−0.015 and M_{Compounds}=1.36 for dendritic mitochondrial content (E), and M_{DMSO}=0.046 and M_{Compounds}=−1.01 for median axonal mitochondrial circularity (F). G) Elongation hits increase dendritic mitochondrial length. Relative frequency distributions of dendritic mitochondrial length (left panel) and the average of their median values (right panel, medians_{DMSO}=3.39±0.08 μm, medians_{Hit}=3.62±0.14 μm). H) Mitochondrial content hits increase dendritic mitochondrial CA. Frequency distribution histograms of individual mitochondrial areas (left panel) and average CA values (right panel, mean_{DMSO}=936±183 μm², mean_{Hit}=1467±397 μm²). I) Health hits reduce axonal mitochondrial circularity. Frequency distribution histograms of axonal mitochondrial circularities (left panel) and averages of their median values (right panel, medians_{DMSO}=0.85±0.0185, medians_{Hit}=0.82±0.01). G-I bargraphs are mean±s.d. of 10 well values from the 10 replicate plates (n_{compound}=1 well/plate, n_{DMSO}=42 wells/plate), frequency distribution data were collected from 4 fields/treatment, total mitochondria n_{DMSO,Elongation}=1741, n_{Hit,Elongation}=3074, n_{DMSO,Hit Content}=1365, n_{Hit,Mt Content}=2782, n_{DMSO,Health}=4262, n_{Hit,Health}=4779. Unpaired t-test ** p<0.01, *** p<0.001. J-K) Representative images of compounds that increase elongation, mitochondrial content or improve health. Z-projections of fluorescent images of primary neurons expressing Mt-GFP were collected using a 60× objective. Scalebars=5 μm (J); 20 μm (K). L) Hit enrichment during the screen and hit distribution among mitochondrial parameters. The primary screen of 2400 compounds produced 149 potential hits (6.2%), of which 67 were confirmed in the rescreen (45%). Thirty-five of the confirmed hits were hits for more than one parameter.

FIG. 4. Mitochondrial Dynamics and Function. (A) Uncorrelated mitochondrial content and neurite sprouting. Rescreen data of dendritic mitochondrial CA and neurite area of the compounds that significantly increased mitochondrial content (>2 robust Z-scores, dashed line). There was no correlation between increased neurite area and increased dendritic mitochondrial CA (Pearson's r=0.13, right panel). Eleven compounds that increased mitochondrial content (gray) increased neurite sprouting (light gray) by >2 robust Z-scores. (B) No correlation between dendritic and axonal mitochondrial content. There was no correlation between axonal and dendritic CA (Pearson r=0.22). (C) Coupling of dendritic mitochondrial content and length. Dendritic mitochondrial length correlated with dendritic mitochondrial content (left panel, Pearson r=0.76) and dendritic mitochondrial count (right panel, Pearson r=0.6) after compound treatment. The correlation lines are bounded by a 95% confidence interval (gray shaded area). (D) Representative images showing the coupling of mitochondrial content and elongation. Mt-GFP expressing neurons were treated with a hit compound (12.5 μM) that increased both content and elongation by 12 h after treatment. White arrows indicate the sites of mitochondrial growth. Scalebar is 20 μm. (E) Axonal mitochondrial length is uncoupled with content and count. There was no correlation between axonal mitochondrial length and axonal CA (left panel, Pearson r=−0.22) or count (right panel, Pearson r=−0.23). (F) Mitochondrial dynamics hits increase mitochondrial function. TMRM signal and ATP production of the rescreened mitochondrial dynamics hits (confirmed hits, n=67) was measured and a functional increase was determined as robust Z-scores >2 for TMRM signal or ATP production. Sixty-one increased (91%) one or both functional readouts. (G) Mitochondrial dynamics hit parameters differentiate compounds with increased mitochondrial function. Decreased axonal circularity, increased dendritic length together with increased dendritic CA identified a population of compounds showing increased mitochondrial function measured by ATP production, TMRM signal or both (functional increase, darker points, >2 robust Z-scores, average from 4 replicate plates) among the rescreened primary hits (n=149). (H) Distribution of the 67 rescreened compounds across content, health, elongation and function. Venn diagram of compounds that significantly increased ATP production or TMRM signal (n=61), mitochondrial content (n=53), elongation (n=50), and/or health (n=42). Significance threshold: 2 robust Z-scores.

FIG. 5. MnMs Protect from Oligomeric Aβ(1-42), Peroxide and Glutamate Induced Mitochondrial Damage in Primary Neurons. (A) Effects of the selected MnMs. Parameters were measured 24 h after treatment with 12.5 μM of compounds. data are mean robust Z-scores from FIG. 4 (B) Effects of selected MnMs in the presence of disease-related insults. Representative images of mitochondrial fragmentation induced by 10 μM Aβ(1-42), 75 μM peroxide (PO) or 25 μM glutamate (GLUT) in Mt-GFP expressing neurons, and the mitochondrial protection by 12.5 μM compounds after 48 h of co-treatment (top left, scalebar=20 μm). After co-treatment, axonal mitochondrial circularity (health), axonal mitochondrial average area, dendritic mitochondrial length (elongation) and live-dead cell ratio (survival) were either protected (within 2 Z-scores of vehicle treated control), improved (>2 Z-scores for axonal mitochondrial area, elongation and survival and <−2 Z-scores for health) or were damaged (<−2 Z-scores for axonal mitochondrial area, elongation and survival and >2 Z-scores for health) compared to the vehicle treated control (top right and bottom panels). Compounds fully protective (all parameters are protected or improved) against an insult are bolded. Color bar represents absolute Z-score values either towards improvement (top three gray scale shades) or damage (bottom three gray scale shades) of the parameters. Data are mean Z-scores (n=12-18 wells, 4 fields/well, 2 independent experiments). (C) Selection of the top 7 MnMs. Summary of the steps for the selection of the top 7 compounds.

FIG. 6. MnMs Enhance ATP Production from Isolated Mitochondria, Potentiate Basal Synaptic Activity, and Increase Respiration of Mitochondria In Vivo. (A) Kinetics of ATP production of isolated mitochondria. Baseline-corrected average curves of the ATP production of isolated forebrain mitochondria of newborn C57BL/6J mice starting 20 min after the addition of vehicle (DMSO), 12 μM of FCCP and the 7 top MnMs. Data are mean±s.d. of 4 runs from 1 experiment. Single exponential fits yielded amplitudes of 22498±358 (DMSO, R²=0.9), 17958±216 (FCCP, R²=0.98), 28383±278 (alverine, R²=0.96), 28882±673 (dyclonine, R²=0.91), 49917±838 (naftopidil, R²=0.98), 22764±315 (orphenadrine, R²=0.91), 9853±132 (2′,4′-dihydroxychalcone, R²=0.92), 6121±99 (4′-hydroxychalcone, R²=0.9), 10059±166 (rhamnetin, R²=0.9). (B) ATP production of isolated mitochondria of the top 7 MnMs. Average ATP production of forebrain mitochondria of newborn mice in the presence of DMSO, 12.5 μM FCCP or 12.5 μM of the top 7 MnMs, normalized to average DMSO treatment. Data are mean±s.e.m. of 3 independent experiments (4 runs/experiment), comparison to DMSO by one-way ANOVA with Dunnett's post hoc, * p<0.05, ** p<0.01, *** p<0.001, **** p<0.0001. (C, D) Synaptic activity of hippocampal neurons. Example traces (C) and quantified sEPSC amplitude/frequency (D) of cultured hippocampal neurons at DIV8 after 24 hr of incubation with DMSO, 12.5 μM dyclonine, alverine or naftopidil using whole-cell patch-clamping techniques. Data are mean±s.e.m. (n=33-36 cells) compared to DMSO by one-way ANOVA with Dunnett's post hoc, * p<0.05, ** p<0.01, *** p<0.001, **** p<0.0001. (E, F) Mitochondrial respiration in primary neurons exposed to dyclonine for 24 hr. Prior to oxygen consumption rate (OCR) measurements, DIV13 neurons were treated for 24 hr with DMSO (0.1%, black) or dyclonine (10 μM, gray). OCR rates were measured at baseline respiration (Base) and after addition of mitochondrial substrates Pyruvate (P) and Malate (M) or Succinate (Succ) along with ADP to the permeabilized (Saponin, Sa) neurons (1′ dashed line, 9) Oligomycin addition (2^{nd} dashed line) blocked proton re-entry through ATP synthase, slowing ETC function to levels necessary to maintain Δψₘ (S4ₒ; o=oligomycin induced). Addition of FCCP (3^{rd} dashed line) short-circuited proton influx to bypass ATP synthase, revealing maximal, uncoupled OCR (S3_{U}; u=uncoupler induced). Addition of Rotenone (R)/Antimycin A (AA), Complex I and Complex III inhibitors, respectively, were added to block oxidative phosphorylation and measure non-mitochondrial respiration (4^{th} dashed line). (G, H) Respiration of isolated mitochondria from dyclonine treated mice. OCR of whole brain mitochondria of 9-month old C57BL/6J mice kept on either dyclonine-supplemented (25 mg/kg) or standard water for 7 months was measured in the presence of complex I (G) or complex II (H) substrates preincubated with mitochondria, yielding similar results as in E and F. Bar graphs (right panels of E-H) compare the OCR value of the first measured point in each condition between the water- and dyclonine-treated groups (mean±s.e.m., n=11-12 wells). Total protein was used to normalize OCR into pmol O₂/min/ug total protein. Data in panels E-H were analyzed using by two-way ANOVA with repeated measures. * p<0.05, ** p<0.01, *** p<0.001, **** p<0.0001.

FIG. 7. Optimizing the Screen (A) AAV9 vector containing the Cre-recombinase gene. Plasmid map of the vector containing the CaMKII promoter and the iCRE sequence fused with tdTomato via the T2 peptide sequence. The expression of td-Tomato from the virus in the cytoplasm of neurons was negligible and not used for the experiments shown. (B) Time course of Mt-GFP expression in primary neurons. Representative images of a neuron expressing Mt-GFP (upper panels) on different days of culture in vitro (DIV). Normalized fluorescence of Mt-GFP intensity over time showed that on DIV14 the signal was 1.6±0.16 times more intense than the background, which plateaued at 2.3 defined by the sigmoidal fit (green line). (C) Correct mitochondrial targeting of the Mt-GFP reporter. Mt-GFP expressing neurons were stained with the mitochondrial dye TMRM (left panels) or MitoTracker Orange (MTO, image not shown). The overlapping Mt-GFP and TMRM signals (white box) are separated and enlarged in the trio of images to the right. Quantification of the overlap was performed by manually measuring the area of individual Mt-GFP expressing mitochondria (n_{TMRM}=61, n_{MTO}=70) and the area of the corresponding TMRM or MitoTracker Orange signal using ImageJ, validated by linear fits to the data (R²_{TMRM}=0.94, R²_{MTO}=0.95). (D) Candidate screen for positive controls. Twenty-three compounds at 4 concentrations were added with a pintool to 384-well plates on DIV13 and imaged on DIV14. After image and data analysis, none of the compounds increased mitochondrial content (dendritic CA), elongation (dendritic length median) or improved health (axonal circularity median) by more than 2.5 Robust Z-scores compared to DMSO treated neurons. Data are means of 4 well replicates per concentration, 4 fields/well.

FIG. 8. Mitotoxicity and Neurotoxicity with FCCP treatment. (A) Mitotoxicity. Mitochondrial health was assessed by measuring axonal mitochondrial circularity with increasing concentrations of FCCP treatment (0-12.5 μM) on DIV13 and collecting data 24 h later. The correlation between axonal mitochondrial circularity and FCCP concentration is linear (R²=0.99). Data are mean±s.e.m. (12 wells, 4 fields/well) fit with a linear function. (B) Neurotoxicity. The fraction of dead cells was measured 24 h after treatment of neurons with increasing concentrations of FCCP (0-12.5 μM) on DIV13. The correlation between dead cells and FCCP concentration shows that there is an initial lag before cells begin to die with FCCP treatment. Data are mean±s.e.m. (6 wells, 6 fields/well) fit with a sigmoidal function constrained at a ceiling of 1 as a theoretical limit for the dead cell fraction. (C) Mitotoxicity and neurotoxicity. The fraction of dead neurons plotted against axonal circularity of mitochondria with increasing concentrations of FCCP treatment. The correlation depicts an initial lag, with mitochondrial damage occurring before neurons start to die. Data are mean values from A and B fit with a sigmoidal function.

FIG. 9. False Positive Hit Rate in the Screen. (A) Example plate heatmap. Neurons were cultured for two weeks and imaged on DIV14 without compound treatment. Robust Z-scores of well values for mitochondrial content and elongation showed 4 false positive hits on the plate (above 2.5 robust Z-scores). Greater edge effects were observed on this plate for the health parameter yielding 10 false positive hits (2.6%, below −2.5 robust Z-score). During the screen, columns 1, 2, 23 and 24 were omitted from analysis, and rows A, B, O and P were analyzed as a separate group. (B) Averaged plate heatmaps. Averaging the robust Z-scores of each well at the same position of 4 the replicate plates used during the screen resulted in no false positive hits for any of the hit parameters.

FIG. 10. Structural Clustering of the MnMs. Tanimoto similarities between the Morgan fingerprints of the rescreened hits were calculated and the molecular structures were clustered by the WPGMA (Weighted Pair Group Method with Arithmetic Mean) method. Compounds that were hits for a parameter are differently colored in the same dendograms mitochondrial content—blue (A), elongation-magenta (B), health—green (C). Dendrograms show that hit compounds for the 3 parameters do not form distinct structural clusters.

FIG. 11. Dose-Response of Aβ(1-42) Oligomers, Peroxide and Glutamate Treatment on Neuronal Mitochondrial Features. A-C) Dose response of Aβ(1-42) treatment. Sigmoidal dose response curves fit to increasing concentrations (0-15 μM) of Aβ(1-42) showed that oligomer treatment led to smaller (A) and more circular (B) axonal mitochondria. Aβ(1-42) treatment also decreased the average length of dendritic mitochondria (C). Data are means±s.e.m. (10 wells, 4 fields per well, 2 independent experiments) collected 48 h after Aβ(1-42) oligomer treatment on DIV13. D-F) Dose response of peroxide treatment. Sigmoidal dose response curves fit to increasing concentrations (0-800 μM) of peroxide showed that peroxide treatment was very similar to Aβ(1-42) treatment, leading to smaller (D) and more circular (E) axonal mitochondria, and shorter dendritic mitochondria (F). Data are means±s.e.m. (12 wells, 4 fields per well, 2 independent experiments) collected 48 h after peroxide treatment on DIV13. G-I) Dose response of glutamate treatment. Sigmoidal dose response curves fit to increasing concentrations (0-200 μM) of glutamate showed that glutamate treatment was very similar to peroxide treatment, leading to smaller (G) and more circular axonal mitochondria (H), and shorter dendritic mitochondria (I). Data are means±s.e.m. (12 wells, 4 fields per well, 2 independent experiments) collected 48 h after peroxide treatment on DIV13. J) Glutamate induced neuronal death. Live cell ratio (live cells/live+dead cells) was determined at increasing concentrations of peroxide or glutamate treatment of neurons on DIV13 after incubation for 48 h. Peroxide (75 μM) and glutamate (25 μM) treatment caused 46% and 42% death among neurons compared to control (live cell ratio decreased from 0.57 to 0.24 and 0.26 for peroxide and glutamate treatment, respectively). Data are means±s.e.m. (6 wells, 8 fields per well, 2 independent experiments) collected 48 h after peroxide or glutamate treatment on DIV13. There was no detectable death of neurons with 10 μM Aβ(1-42) treatment for 48 h.

FIG. 12. Scheme of field outlier removal and using neutral wells for robust Z-score calculation.

FIG. 13. Development of the high-throughput TMRM and ATP assays. (A) TMRM fluorescence. The excitation and emission spectra of TMRM. (A′) TMRM fluorescence as a function of concentration in primary neuronal cultures. DIV14 neurons were loaded with TMRM with serial concentrations from 10 to 160 nM. After 6 h of incubation, neurons were washed briefly, and the intra-mitochondrial fluorescence was measured. TMRM fluorescence increased linearly with increased concentration below 40 nM in the medium. High concentrations within mitochondria triggered self-quenching effects and a deviation from linearity. The mitochondrial TMRM fluorescence across TMRM concentration was fit to a hyperbolic function. Bar plots represent the mean±the SEM. (B) Quenching and non-quenching concentrations of TMRM in primary neuronal cultures. DIV14 neurons were loaded with TMRM at 10, 20, 50 and 150 nM for 3 h and then treated for 5-10 m with DMSO (0.1%), the ionophore FCCP (5 and 10 μM) or the ATP synthase inhibitor oligomycin (5 and 10 μM). Cells were washed and the intracellular TMRM intensity was measured. A ΔΨₘ hyperpolarization by oligomycin triggered further accumulation of mitochondrial TMRM, thus a further self-quenching effect and decreased fluorescence at high concentrations (150 nM), but increased fluorescence at non-quenching concentrations (10 and 20 nM). In contrast, FCCP collapses ΔΨₘ and releases mitochondrial TMRM, producing a transient increase of fluorescence at quenching concentrations (150 nM) and a decrease in fluorescence at non-quenching concentrations (10 and 20 nM). Bar plots represent the mean of well fluorescence intensity ±the SEM normalized to DMSO-treated wells (n=12). One-way ANOVA followed by Dunn's post hoc for each concentration of TMRM. * P<0.05, ** P<0.01 and *** P<0.001 compared to each DMSO control. (C) TMRM assay wash steps do not affect the integrity of the cultured neurons. Representative images of TMRM labeled primary neurons before and 0, 15, and 30 m after washing steps, showing that the wash steps neither disturb the cell layer nor alter the morphology of neurons and mitochondria. Scale bar=20 μm. (D) TMRM wash steps are required for an accurate determination of mitochondrial associated TMRM fluorescence. The raw (left) and normalized (right; normalized to each of the DMSO controls) TMRM signal in untreated, DMSO- and FCCP-treated wells, pre- and post-washing steps. Wash steps remove about 40% of the total fluorescence due to the removal of free TMRM in the medium, indicating that approximately 60% of the TMRM is sequestered in mitochondria. FCCP collapses ΔΨₘ, strongly reducing the mitochondrial signal in the washed wells but not in those measured before wash. Bar plots represent the mean±the SEM. (E) Distribution of TMRM fluorescence in neurons treated with positive hits (red), DMSO (black) and the negative control, FCCP (gray). DIV13 neurons were incubated with DMSO (0.1%), FCCP (12.5 μM) and the selected hits, dyclonine (12.5 μM) or doxepin (12.5 μM), for 24 h and the ΔΨₘ measured by whole well TMRM fluorescence. The results are presented as % TMRM fluorescence relative to the in-plate DMSO control which was normalized to 100%. n=128 wells for each group; *P<0.05, **P<0.01, ***P<0.001, ****P<0.0001 compared with DMSO-treated controls, one-way ANOVA followed by Dunn's post-hoc. (F) Plate edge effects. In a pilot experiment using two plates with wells treated only with the carrier DMSO, we observed significant edge effects on TMRM fluorescence, with elevated signal in the outer two rows or columns but relatively homogenous signals across the population of inner wells. Heat maps of TMRM fluorescence from columns 2-23 of DMSO treated plates at 4 and 24 h of incubation. The fluorescence is the average per well across two independent plates. Primary neurons in 384-well plates were cultured to DIV13 and incubated with TMRM (10 nM) and DMSO (final concentration 0.1%) in all wells. The TMRM intensity was assayed in a plate reader at 4 and 24 h after the necessary washing steps. No TMRM dye was added in columns 1 and 24 (not illustrated). The auto-fluorescence of the neurons and plastics in the plate bottom in these wells was measured as background and subtracted from the wells to which TMRM was added. The black box represents the “inner wells” used for the screen. (G) TMRM plate row and column effects. The average whole well TMRM fluorescence across rows and columns at 4 and 24 h. Significant edge-effects were observed across rows A-P, with the outer two rows exhibiting significantly increased TMRM fluorescence relative to inner rows. Data plotted as mean±std. To compensate for the significant edge effects, data generated from the outer rows (rows A,B,O,P) and inner wells (rows C-N, columns 3-22) were analyzed separately for the primary screen as described below. The outer rows A, B, O and P were not used for the subsequent rescreen and dose response experiments. Bar plots represent the mean±the SEM. (H, I) TMRM assay variability across plates. The MicroSource Spectrum library compounds were pintooled from source plate rows A-P, columns 3-22, and DMSO from a source plate rows A-P, columns 2 and 23. The basal TMRM fluorescence was measured from 32 DMSO (0.1%) control wells in columns 2 and 23 on each plate to monitor variability across the screen. There was no significant difference in average TMRM fluorescence of the DMSO-treated wells at 4 (H) or 24 h (I) of incubation across the four replicates for each of the eight source plates (Spec 1-8). In addition, no significant signal drift was observed from the first to the last plate throughout the primary screen indicating consistency in the primary neuron cultures and assay. Points represent individual wells and the bars represent the mean±std. The red horizontal line indicates the basal TMRM fluorescence at 4 (H) or 24 h (I) of incubation, measured from DMSO-treated wells and calculated as the mean across all plates used in the primary screen. This mean showed a ˜10% reduction between 4 and 24 h of incubation. (H′-I′) Representative correlation plot of TMRM signals for compound-treated wells of replicates plates 1 (P1) and 2 (P2) for Spectrum source plate 4. The table inset shows the high correlation (R values >0.92) for all inter se comparisons among replicate plates. (H″, I″) The Coefficient of Variation (CV) of four replicates for each of the 2400 compound-treated wells was individually plotted to illustrate variation across replicates. The CV was centered around 3% and was generally <8% for almost all nontoxic compounds. Higher CV values were observed in wells treated with toxic compounds (red). (J,K) Population control for data normalization of TMRM fluorescence. We used a “non-DMSO-control” statistical approach to normalize raw TMRM fluorescence into Z scores and Effect-Size (fold change) for hit identification, based on the fact that the majority of small molecules in HTS assays are absent of biological effects, thus serving as “within plate controls” (Brideau et al., 2003; Malo et al., 2006). Distribution of TMRM fluorescence from 240 inner wells treated with compounds from source plate 4 at 4 (J) and 24 h (K) showing that most of the wells fall into a normal distribution and can be considered to consist of inactive compounds. Some compounds in the inner wells dramatically reduced the TMRM signal and were classified as toxic compounds; others were identified as auto-fluorescent. The wells containing these compounds were not included in the “inactive” population. The plots in (J′, K′) expand the red-highlighted region in (J, K). A bimodal distribution (red line), most obvious in the 24 h data set was observed, identifying putative hits in the shoulder peak. The TMRM fluorescence of “inactive” compounds within the dominant peak showed a similar plate mean (μₚₗₐₜₑ) and standard derivation (σₚₗₐₜₑ) calculated from inner DMSO-wells in parallel DMSO-only treated plates. This “inactive” population from each plate was used for data normalization. Statistically, this subpopulation was defined by using Tukey's fences rule based on the interquartile range (J′, K′). The black dotted line shows the normal distribution of the “inactive” subpopulation with TMRM readouts in the range of [Q1-(Q3-Q1/ITQ), Q3+(Q3-Q1)]. Compounds with activity outside of this range were identified as outliers; Q1 and Q3 represent the first (lower) and third (upper) quartiles respectively. A modified box-and-whiskers plot is illustrated below each distribution. Boxes represent the interquartile range, lines within boxes are medians, whiskers represent values of 1.0×(interquartile range/ITQ: Q3-Q1) from the first (Q1) and third (Q3) quartiles. (L-N) TMRM assay Z-scores and effect size. The plate mean (μₚₗₐₜₑ) and standard deviation (σₚₗₐₜₑ) were calculated using the subpopulation trimmed of outliers to normalize compound performance as Z-scores and Effect Size. Z-scores were calculated by subtracting the μₚₗₐₜₑ, then dividing by the σₚₗₐₜₑ ((value_{compound}−μₚₗₐₜₑ)/σₚₗₐₜₑ). Effect size was calculated by subtracting the μₚₗₐₜₑ, then dividing by the μₚₗₐₜₑ to obtain fold change ((value_{compound}−μₚₗₐₜₑ)/μₚₗₐₜₑ) Putative hits from the primary screen were identified as enhancing the well fluorescence above the μₚₗₐₜₑ by 3σₚₗₐₜₑ for inner wells, and by 2.5σₚₗₐₜₑ for outer wells (FIG. 2E-F). (O) Schematic illustration of the luminescence-based ATP assay in the 384-well plate format. (P-P″) Testing the assay using a DMSO source plate. 100 nl DMSO (final concentration ˜0.1%) was pintooled into the inner wells of a 384 well microplate. Cellular ATP was measured using the CellTiter-Glo® luminescence assay (Promega) and the well luminescence measured. The signal of DMSO-treated wells was aggregated by column (P′) or by row (P″) and plotted. No significant difference in the average luminescence of DMSO wells across the inner columns or rows (one-way ANOVA) was found. Bar plots represent the mean±the SEM. (Q) Orthogonal screen of putative TMRM hits measuring ATP production. 16 putative hits with effects at 4 h (red), and 135 putative hits with effects at 24 h (pink) from the primary screen were tested for ATP production alongside 134 randomly selected non-hit compounds (black) and 40 DMSO controls. The main plot shows the correlation of the effect size of TMRM fluorescence and ATP production in wells after 4 h of incubation for these compounds. The black line represents the fit using a linear regression model using all plotted compounds (R²=0.16, p<0.0001). The compound-induced effect size on ATP production (right histogram) at 4 h was normalized to the mean value of the 40 “within-plate” DMSO controls as (value_{compound}−μ_{DMSO})/μ_{DMSO}. The population of 24 h TMRM hits was significantly different from the non-hit population for ATP effect size (right histogram). The 24 and 4 h TMRM hit populations were significantly different from the non-hit population for TMRM fluorescence effect size (bottom histogram). The 4 h TMRM hits showed a large variation in their effect size on ATP levels (note the distribution of 4 h TMRM points), suggesting that acute increases in ΔΨₘ only weakly correlate with effects on ATP generation. One-way ANOVA followed by Dunn's post hoc to compare among groups, confidence levels were set at *P<0.05, **P<0.01, ***P<0.001.

FIG. 14A to FIG. 14E′. Dose response results for functional modulators of neuronal mitochondria. Dose response assays of compounds in clusters with structural similarity. The 2D-structures of the compounds are displayed with the common core scaffold highlighted in red. We used 15 concentrations from 6 nM to 100 uM for dose response assays. Points represent the mean±SEM of 6 independent experiments, with each compound tested in duplicate in each experiment. EC₅₀ values were calculated from the fitted logistic curve. A summary of the dose response curves for compounds tested for both TMRM fluorescence and ATP generation is illustrated at the left for each group for visualizing and comparing the magnitude of TMRM and ATP effect size.

FIG. 15. Additional dose response results for functional modulators of neuronal mitochondria. Dose response assays of compounds in clusters with structural similarity. The 2D-structures of the compounds are displayed with the common core scaffold highlighted in red. We used 15 concentrations from 6 nM to 100 uM for dose response assays. Points represent the mean±SEM of 6 independent experiments, with each compound tested in duplicate in each experiment. EC₅₀ values were calculated from the fitted logistic curve. A summary of the dose response curves for compounds tested for both TMRM fluorescence and ATP generation is illustrated at the left for each group for visualizing and comparing the magnitude of TMRM and ATP effect size.

FIG. 16. TMRM and ATP assays and screens for small molecule modulators of neuronal mitochondrial function. (A-A″) Primary mouse cortical neurons loaded with TMRM and imaged 24 h later. The two left-most images (20×) show the cell density at DIV14 (A) and fluorescent mitochondria in cell bodies and neurites (A′); the image (60×) at the right (A″) illustrates neuronal mitochondria at a higher power including the intense, peri-nuclear mitochondria. n=nucleus. Scale bar=400 μm for A-A′ and 20 μm for A″. (B) High-throughput screening pipeline for small molecule enhancers of neuronal mitochondrial function. Primary screen: Primary neurons were isolated, plated and cultured to DIV13, and loaded with TMRM before compounds were added with a pin-tool. At 4 and 24 h after compound addition the neurons were washed and TMRM fluorescence was read. Orthogonal screen: TMRM hits selected from the primary screen were rescreened and tested for their ability to enhance neuronal ATP production in an orthogonal screen. Functional Analysis: TMRM/ATP positive hits were clustered by structural similarity. Representative hits from main structural clusters were further assayed for dose response, effects on oxygen consumption (OCR), mitochondria morphology, neurite sprouting and protection to neurons against insults associated with neurodegenerative diseases. (C-D) Scatter plots of normalized Z-scores (Z) of TMRM fluorescence, (C′, D′) as zoomed in (orange shading in C,D) to eliminate most toxic and auto-fluorescent reads, and (C″, D″) effect size of TMRM signals at 4 (C-C″) and 24 h incubation (D-D″). Z-score=(value_{compound}−ₚₗₐₜₑ)/σₚₗₐₜₑ). Effect size=(value_{compound}−μₚₗₐₜₑ)/μₚₗₐₜₑ). Z-score and Effect-size (E) values for each compound were calculated per-plate using population statistics from four replicate plates. We used the population of “inactive” compound-treated wells as the control; see FIGS. 13J and 13K for details on the “non-DMSO-control” statistical approach. Results are plotted as means±SEM from the quadruplicates. Putative hits that increased intracellular TMRM intensity above the plate mean (μₚₗₐₜₑ) by 3σ for the inner wells, and by 2.5σ for the outer wells were selected for re-screen. Distribution histograms of Z-score and Effect size are shown at the right side of panels C′, C″, D′, D″. Arrowheads indicate the subpopulation identified as putative hits. (E, F) Orthogonal screen of putative TMRM hits measuring ATP production. 135 putative hits with effects at 24 h (red) from the primary screen were re-tested alongside 134 randomly selected non-hit compounds (black) and 40 DMSO controls (gray). The compound effect at 24 h incubation on TMRM fluorescence and ATP production was normalized to DMSO controls on the same plate in the rescreen. Data were calculated as (value_{compound}−μ_{DMSO})/μ_{DMSO}. The 135 putative 24 h hits (red) plus 134 non-hits (black) are shown in the plot. Each point represents the mean from four replicate wells. (E) Correlation plot of 24 h TMRM fluorescence from the primary screen and rescreen. Linear regression fitted to values generated from the plotted 269 compounds (black line, R²=0.63). The distribution of normalized TMRM effect-size in wells treated with putative hits (red), non-hits (black) and DMSO (gray) is shown along the abscissa. One-way ANOVA followed by Dunn's post hoc was used for comparisons with the DMSO control and non-hit groups. ****P<0.0001. (F) Correlation plot of compound effects on ATP production and TMRM fluorescence. The distribution of compound effect size on TMRM fluorescence and ATP production after 24 h of incubation is shown in the histograms across the abscissa and ordinate, respectively. The putative 24 h TMRM hits are labeled in red, non-hits in black and DMSO-treated wells in gray. Linear regression fitted to values generated from the plotted 269 compounds (black line, R²=0.46). One-way ANOVA followed by Dunn's post hoc was used for comparing with the DMSO control and non-hit groups. ****P<0.0001. Compounds were selected as TMRM/ATP hits if the effect size for ATP generation was greater than 15% (>3σ_{DMSO}, dashed lines in E and F) in the orthogonal screen in addition to being confirmed as TMRM hits.

FIG. 17. Hits from the small molecule screen for neuronal modulators of mitochondrial function. Weighted Pair Group Method with Arithmetic Mean (WPGMA) clustering of the 112 confirmed TMRM/ATP 24 h-hits into nine groups based on the Euclidean distance of Tanimoto similarities of their Morgan fingerprints with a cut-off at value 1.45 (red line). Representative chemical scaffolds for some clusters are high-lighted in red. Shading: orange=local/topical anesthetics; green=isoflavones; grey=COX inhibitors; brown=alpha/beta blockers; pink=antipsychotics and tricyclic antidepressants; purple=anti-cholinergics; yellow=anti-dopaminergic/serotonergic compounds.

FIG. 18. Effects of compounds on mitochondrial function. (A-B′) Mitochondrial respiration in primary neurons exposed to compounds identified as mitochondrial functional enhancers from the screen. Prior to OCR measurements, DIV13 neurons were treated for 24 h with DMSO (0.1%) or the compounds (10 μM). Neurons were gently permeabilized using 25 μg/ml saponin (Sa). OCR measured from permeabilized neurons was displayed in the “middle point” mode showing a single OCR rate for each measurement period. Two measurements were performed under each condition (Base=baseline respiration, S3=ADP/substrate-stimulated State 3 respiration, S4ₒ=oligomycin induced State 4 respiration) except for S3. (the FCCP-stimulated uncoupled State 3 respiration) which consisted of only one measurement. The right bar graph in each panel compares the averaged OCR from the two measurements in each condition among the DMSO- and compound-treated groups. The statistics shown were generated using two-way repeated measure ANOVA with the single control and the experimental groups. Sa=saponin, ADP=adenosine diphosphate, P=pyruvate, M=Malate, Succ=succinate, Oligo=oligomycin, FCCP=carbonyl cyanide-p-trifluoromethoxyphenylhydrazone, AA=antimycin, R=rotenone). Note: We recently reported the experimental results for dyclonine (Varkuti et al., 2020), performed in parallel with yohimbine and doxepin as reported here. Thus, the DMSO data shown is replicated from Varkuti et at, 2020. Statistical significance data was obtained from the DMSO control and all three experimental groups (yohimbine, doxepin, dyclonine). (C-D′) Respiration of mitochondria isolated from brain neurons. Brain mitochondria were isolated from C57BL/6J animals administered either compound-supplemented (5 mg/kg yohimbine) or standard water for 7 months starting at 2 months of age. State 3 respiration was stimulated by addition of ADP (1 mM). Complex I substrates pyruvate and malate (P/M) or the Complex II substrate succinate (Succ) were pre-included in the assay buffer at 10 mM before the measurement of baseline respiration to maintain the healthy state of isolated mitochondria. OCR measured from isolated brain mitochondria is displayed in the “point-to-point mode” showing a series of OCR rates across each measurement period. The right bar graph in each panel compared the OCR value of the first measured point in each condition between the water- and compound-treated groups. Total protein was determined using the BCA assay and used to normalize OCR into pmol O₂/min/ug total protein. Results in all panels are plotted as the mean±SEM (n=11-12). Data were analyzed by two-way repeated measure ANOVA followed by Bonferroni's multiple comparison tests. *P<0.05, **P<0.01, ***P<0.001, compared with corresponding controls.

FIG. 19. Effects of compounds on mitochondrial morphology and neurite sprouting. (A-A′″, B) Morphological changes in primary neuronal mitochondria due to treatment with mitochondrial functional modulators. Using fluorescent reporters expressed in the mitochondria (mito-GFP, 2nd column) and cytosol (cytosolic-tdTomato, 3rd column), the circularity and length of mitochondria and the area of neurites in images were quantified after segmentation (e.g. extraction of neuritic area with ImageJ, 4th column). (A-A′″) DIV13 neurons were treated with selected TMRM/ATP hit compounds or DMSO vehicle control and imaged after 24 or 48 h. (B) Neuritic mitochondria, which vary in length, were classed into axonal mitochondria (0.5 μm length 1.4 μm) and dendritic mitochondria (length 2.4 μm) based on our prior studies (Varkuti et al, 2020). The circularity of axonal mitochondria and the length of dendritic mitochondria were measured using the GFP signal and the neurite area from segmenting tdTomato fluorescence. These parameters were normalized as robust Z-scores relative to in-plate, DMSO control wells. Data are presented as means±SEM (n=12 wells).

FIG. 20. Neuronal mitochondrial modulators offer protection against insults associated with neurodegenerative diseases. (A-A″) Mitochondrial modulators protect against the decline in ΔΨₘ caused by increased oxidative stress. Primary forebrain neurons dissociated from C57BL/6J mice were plated into wells of four replicate, 384-well plates at the same density. At DIV13, TMRM was loaded at 10 nM along with 55 μM Luperox and a collection of mitochondrial modulators. Following 24 h of treatment, the fluorescence was measured and normalized as % TMRM fluorescence relative to the within-plate DMSO controls. (A) The dose response effect of t-butyl hydrogen peroxide, a relatively stable peroxide used to increase oxidative stress in cultured neurons. DIV14 neuronal cultures in 384-well plates were treated with sixteen different hydrogen peroxide concentrations from 10 nM to 400 uM, alongside paired DMSO controls and whole well TMRM fluorescence was assayed 24 h later. Points represent the mean±SEM of 3 independent plates, each concentration tested in duplicate/plate. 24 h treatment with Luperox at 50 μM significantly reduced the whole well TMRM fluorescence by ˜40% in DIV14 neuronal cultures. Nearly complete cell death, shown as no intracellular TMRM accumulation was observed in cells treated with Luperox at 100 μM or greater. (A′) Heat map and summarized bar graph (A″) of TMRM fluorescence from untreated and hydrogen peroxide-treated neuronal cultures in response to DMSO (0.1%, control) and mitochondrial functional modulators from five structural clusters. The normalized TMRM fluorescence in the heatmap is the average per well across four replicates. Compounds that provided full neuro-protective effects are those that elevated the ΔΨₘ depression observed in peroxide challenged neurons to the level observed in neurons untreated with Luperox (red dashed line). Compounds at the concentration used that provided full protection to neurons included yohimbine, genistein, dyclonine, benoxinate, doxepin and pimozide. Results are presented as mean±SEM. n=12 wells. (B-B″) Mitochondrial modulators protect against the decline in ΔΨₘ in primary neurons cultures made from 3×TG mice. Primary forebrain neurons dissociated from C57BL/6J (B6) and 3×TG mice were plated into wells of the upper and lower halves of four replicate, 384-well plates at the same density. At DIV22, TMRM was loaded at 10 nM and the modulators were added. Following 24 h treatment, the well fluorescence was measured and normalized to total protein content to correct for potential differences in cell and/or neurite density between B6 and 3×TG neuronal cultures. (B) A significant reduction (˜20%) in average normalized TMRM fluorescence was detected in the 3×TG cultures compared to the co-plated B6 neurons when treated only with 0.1% DMSO carrier. (B′) Heat maps and summarized bar graphs (B″) of TMRM fluorescence from both B6 and 3×TG neuronal cultures in response to DMSO (0.1%, control) and the battery of mitochondrial functional modulators. The normalized TMRM fluorescence in the heatmap is the average per well across four replicates. All compounds provided full neuroprotective effects, indicated as those that elevated the ΔΨₘ depression observed in 3×TG neuronal cultures to the level observed in B6 neurons treated with DMSO (red dashed line). Results are presented as mean±SEM (n=24 wells). Data (A″, B″) were analyzed by one-way ANOVA followed by Dunnett's multiple comparison tests. ***P<0.001 (red), comparing drug-treated wells in the presence of the insult with control wells without the insult. **P<0.01, ****P<0.0001 (black), comparing drug-treated wells to control wells in the presence of the insult.

FIG. 21. Graphs showing in vivo effects of Rhamnetin, in comparison to DMSO control, on axonal mitochondria in a Drosophila fly model of Alzheimer's disease.

### DETAILED DESCRIPTION

### Definitions

In this description, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a “therapeutically effective amount” with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent. A therapeutically effective amount also is the minimum amount necessary to achieve one or more of the mitoprotective results as described herein.

Generally, the initial therapeutically effective amount of a compound described herein or a pharmaceutically acceptable salt thereof that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of the compound or a pharmaceutically acceptable salt thereof. In another embodiment, such dosage forms contain from about 50 mg to about 500 mg of the compound or a pharmaceutically acceptable salt thereof. In yet another embodiment, such dosage forms contain from about 25 mg to about 200 mg of the compound or a pharmaceutically acceptable salt thereof. In still another embodiment, such dosage forms contain from about 10 mg to about 100 mg of the compound or a pharmaceutically acceptable salt thereof. In a further embodiment, such dosage forms contain from about 5 mg to about 50 mg of the compound or a pharmaceutically acceptable salt thereof. In any of the foregoing embodiments the dosage form can be administered once a day or twice per day.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human) In one embodiment, a patient is a human, such as a human infant, child, adolescent or adult. In the present disclosure, the terms “patient” and “subject” are used interchangeably.

In various embodiments, the present disclosure relates to an assay platform for multiplexed, high throughput and phenotypic screening of small molecules for modulatory effects on neuronal mitostasis, focusing on the neuron's content of mitochondria, mitochondrial elongation and health. The latter parameter, health, can be assayed using an imaging function of “circularity.” When mitochondria become defective, they assume a small and rounded morphology. In some examples, we used the platform to screen 2400 compounds and identified 67 Modulators of neuronal Mitostasis, abbreviated as “MnMs,” that modulate mitochondrial content, elongation, and health. Interestingly, more than 90% of the MnMs also increased mitochondrial function as measured by the potential across the inner mitochondrial membrane and ATP generation by the cells, suggesting an extremely high correlation between mitochondrial morphology and function. Importantly, some of the MnMs proved to be protective to cultured neurons against insults that are associated with neuropsychiatric disorders, including increased oxidative stress, excess glutamate or Aβ(1-42) induced mito- and neurotoxicity. Using isolated mitochondria, we found that some MnMs which fully protected damaged mitochondria from these insults, target mitochondria directly. One promising compound, dyclonine, was explored in more detail and shown to enhance synaptic activity in cultured hippocampal neurons and increase respiration in brain mitochondria isolated from mice treated with the compound.

The present disclosure further relates to a high-throughput assay and screen using cultured primary neurons to identify compounds that increase mitochondrial function. As illustrated in various embodiments herein described, the assay identified multiple structural and functional clusters of compounds that enhance the function of neuronal mitochondria. The most surprising observation made concerns the large number and diversity of the compounds identified that potentiate mitochondrial function. For example, the modulators identified in the assay include the hormone derivatives yohimbine and genistein, which provide beneficial effects on mitochondrial function and overall neuronal health. These compounds are exemplary positive controls for use in high-throughput screens that assay mitochondria function. Additional classes of hits identified by the assay include topical/local anesthetics as represented by dyclonine, classic tricyclic antidepressants as represented by doxepin, and the anti-psychotics as represented by pimozide and penfluridol. These results offer evidence that these molecules and classes can offer protection against diseases that are associated with neuronal mitochondrial dysfunction. The present disclosure, as illustrated by various embodiments, establishes methods of screening large libraries of compounds for their effects on mitochondrial function directly in neurons, providing a more biologically relevant HTS platform for use in brain disease research.

Thus, in one embodiment is an in vitro method for determining whether a test agent could be useful as a mitotherapeutic in the treatment of a patient suffering from a neurological or psychiatric disorder. The method comprises (a) contacting a test population of brain cells with a mitochondrial reporter for a time sufficient to label mitochondria in live neurons.

Mitochondrial dyes, labels, probes, reporters, and stains are terms that are used interchangeably in this disclosure. Typical mitochondrial dyes, such as fluorescent dyes, are known in the art. For example, a useful mitochondrial reporter is tetramethylrhodamine methyl ester perchlorate (TMRM). Additional mitochondrial reporters include tetramethylrhodamine ethyl ester perchlorate (TMRE), 6-amino-9-(2-methoxycarbonylphenyl)xanthen-3-ylidene]azanium chloride (Rh123), 5,5,6,6′-tetrachloro-1,1′,3,3′ tetraethylbenzimi-dazoylcarbocyanine iodide (JC-1) and 3,3′-dihexyloxacarbocyanine iodide (DiOC₆). Sufficient times for labeling mitochondria, as can be assessed by straightforward imaging as described herein, can vary from one hour to 72 h. After labeling, according to some embodiments, the cells are isolated from the mitochondrial dye so as to not contaminate a further imaging step as described herein.

In this embodiment and others, the method next comprises (b) incubating the cells with the test agent. Automated high-throughput screening methodologies are useful, for example, in assaying a plurality of test agents. A test agent can be any substance, typically an agent known or suspected to be biologically active, such as any of the small molecule compounds described herein.

The methods disclosed herein further comprise (c) imaging the incubated cells to obtain a visual image of labeled mitochondria. Many imaging techniques and instrumentation, suitably matched to the emission profile of a chosen mitochondrial reporter, are known in the art. One example, per one embodiment, is automated confocal imaging as described herein.

The method further comprises (d) determining mitochondrial parameters by inspection of the visual image, in comparison to an image of a control population of brain cells not incubated with the test agent. Mitochondrial parameters are readily observable or derived quantities facilitated by the imaging. In various embodiments, these are selected from the (1) concentration of cellular mitochondria, (2) mitochondrial length; and, as a measure of mitochondrial health, (3) mitochondrial circularity.

As used herein, the quantity “circularity” can be determined in various ways, such as by visual comparison of mitochondria shapes between the image of labeled mitochondria and control image “Circularity” also can be quantified, in various embodiments, by dividing 4π (area of an individual mitochondria) by (perimeter of the individual mitochondria)². Mitochondrial length and circularity, if quantified, can be independently expressed as their mean and median values. The examples described herein illustrate further embodiments of quantification calculations.

The method further comprises the step of (e) correlating the presence of one or more results with a conclusion that the test agent is useful as a mitotherapeutic. With reference to the mitochondrial parameters described herein, the results are selected from (1) an increase in concentration of cellular mitochondria; (2) an increase in mitochondrial length; and (3) decrease in mitochondrial circularity. In some embodiments, any two of these results are observed. In other embodiments, all three results are observed.

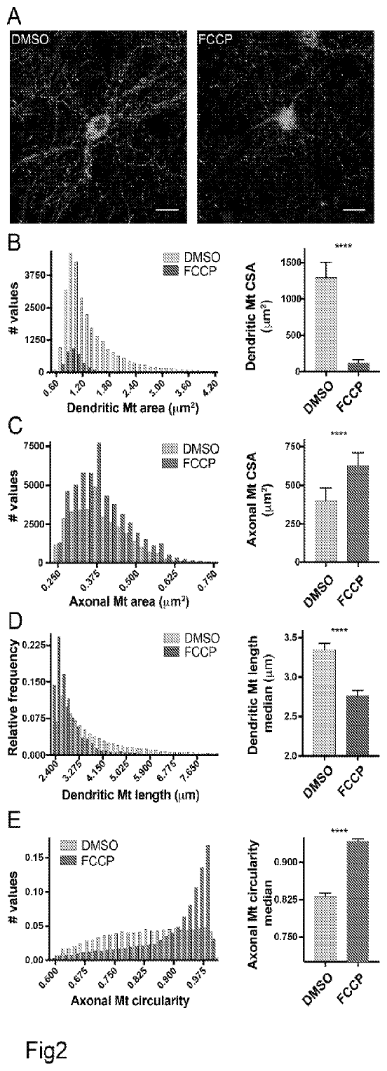

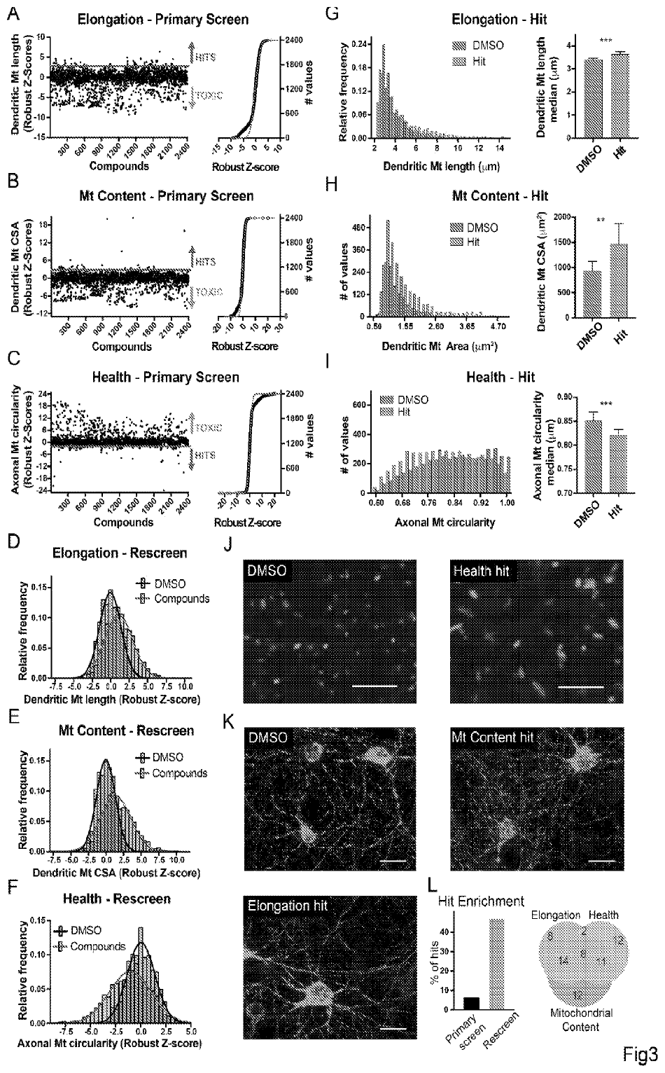

Another embodiment is an in vitro method for determining whether a test agent is likely toxic to cellular mitochondria (see FIG. 2 and FIGS. 3A-3C). Many hazardous agents, e.g., compounds, are found in common or everyday items such as electrical components, industrial chemicals, and agricultural chemicals such as weed killers. In this context, the method is a useful assay for determining likely mitochondrial toxicity of these and other agents suspected of being toxic. The method thus comprises (a) contacting a test population of cells with a mitochondrial reporter for a time sufficient to label mitochondria in live neurons; (b) incubating the cells with the test agent; (c) imaging the cells to obtain a visual image of labeled mitochondria; and (d) determining mitochondrial parameters by inspection of the visual image, in comparison to an image of a control population of cells not incubated with the test agent, wherein the mitochondrial parameters are selected from concentration of cellular mitochondria; mitochondrial length; and mitochondrial circularity. The method further comprises the step of (e) correlating the presence of one or more results with a conclusion that the test agent is likely toxic to mitochondria, wherein the results are selected from a decrease in concentration of cellular mitochondria, decrease in mitochondrial length, and an increase in mitochondrial circularity.

In embodiments, the cells described herein are derived from one of various tissue types. In an embodiment, the cells are muscle cells. In another illustrative embodiment, optionally in combination with any other embodiment, the in vitro methods described herein utilize cells harvested from the brain. Thus, in various embodiments, the cells are brain cells and, accordingly, the cellular mitochondria are neuronal mitochondria. Suitable brains, in various embodiments, are mammalian, such as mouse, rat, pig, and monkey. Additional embodiments provide for cells obtained from brains of other laboratory models including invertebrates, such as insects. In some embodiments, the cells are dissociated from the forebrain.

In various embodiments, optionally in combination with any other embodiment described herein, the concentration of dendritic mitochondria is determined. In additional embodiments, dendritic mitochondrial length is measured. In still further embodiments, mitochondrial circularity is determined by measuring axonal mitochondria. The present disclosure contemplates all combinations of these embodiments.

In another embodiment, the present disclosure provides an in vitro method for determining whether a test agent modulates ATP generation from cellular mitochondria. The method comprises
(a) contacting a test population of cells with a mitochondrial reporter for a time sufficient to label mitochondria in live cells;
(b) incubating the test population of cells with the test agent;
(c) measuring a reporter signal from labeled mitochondria in the test population of cells; and
(d) correlating an increase, no change, or decrease in reporter signal from (c), relative to a reporter signal from a control population of cells not incubated with the test agent, to a determination that the test agent enhances, exerts no effect upon, or impairs, respectively, ATP generation from mitochondria in the test population of cells.

In an illustrative embodiment, optionally in combination with any other embodiment herein described, the mitochondrial reporter is a fluorescent dye, such as TMRM. Thus, in accordance with this choice of mitochondrial reporter, the reporter signal is fluorescence.

The correlating in step (d), per various embodiments, can be facilitated by calculating from the reporter signal an inner mitochondrial membrane potential (ΔΨₘ) that, as explained herein, is a well-known quantity for assessing the functional status and integrity of mitochondria. ΔΨₘ is defined as the difference in electrical potential between the mitochondrial matrix and the cytosol, and is commonly considered as a semi-quantitative read-out for the full proton-motive force. Marked ΔΨₘ depolarization is generally correlated with neuronal death, and it may indicate mitochondrial outer membrane permeabilization during apoptosis or mitochondrial permeability transition during ROS-mediated or Ca²⁺-mediated injury. Small fluctuations in ΔΨₘ can indicate disrupted respiration, ATP synthesis, or ionic fluxes across the mitochondrial membrane.

Incubation times of test agent and test population of cells can range from about 1 h to about 48 h. Exemplary incubation times include at least 4 h, at least 8 h, at least 12 h, at least 16 h, at least 20 h, and at least 24 h. The incubation time can be adjusted to verify ΔΨₘ as a reliable indicator of mitochondrial dynamics and function. Thus, as illustrated by examples and data herein, independent measurement of cellular ATP production can be achieved by incubating a test population of cells with a test agent, and then subjecting the incubated cells to an ATP quantitation assay. Any useful method for measuring cellular ATP can be used, such as the widely known CellTiter-Glo® luminescence assay (Promega). Results from the mitochondrial reporter assay method described herein can be combined with results from the ATP quantitation assay to further screen for test agents that modulate mitochondrial dynamics and function. Thus, for example, an incubation time of about 24 h is suitable for verifying that increased ΔΨₘ is a reliable and inexpensive surrogate for detecting elevated levels of ATP within cells, such as neurons.

Advantages attaching to compounds described herein, such as those identified by the in vitro assay methods described herein, relate generally to their promotion of cellular, e.g., neuronal mitochondrial content, length and bioenergetics or health. More specifically, the compounds (1) Protect neuronal mitochondria from the insults associated with oligomeric Aβ application—Oligomeric Aβ is a toxic peptide that represents one of the universal neuropathologies associated with AD and some other neurodegenerative disorders; (2) Protect neuronal mitochondria from the insults associated with increased oxidative stress—Increased oxidative stress is a universal pathology associated with AD and other neurodegenerative disorders; (3) Protect against increased glutamate toxicity, which is a pathology associated with AD leading to neuronal death; and (4) protect against the toxic cellular environment associated with Alzheimer's disease as reflected in a mouse model for the disease.

The protective advantages of the compounds described herein offer broad protection to the mitochondrial system found in cells of the central nervous system. The mitochondrial system in cells of the central nervous system becomes defective in many different diseases, including Alzheimer's disease, Parkinson's disease, Amyolateral sclerosis, Huntington's disease, and other neurological and psychiatric diseases. These impairments alter the function of individual mitochondria and also mitochondrial dynamics, the latter including the generation of mitochondria by the cell (biogenesis), the turnover of mitochondria (mitophagy) and the division (fission) and fusion that mitochondria undergo with each other.

### Therapeutic Methods and Uses

The present disclosure also provides, in an embodiment, a method for treating a disorder characterized by dysfunction of neuronal mitostasis or dysfunction of ATP generation. The method comprises administering to the patient a therapeutically effective amount of a compound, or a pharmaceutically acceptable salt thereof, as described herein. The method is useful, according to some embodiments, in treating neurodegenerative and neuropsychiatric disorders. Impaired mitochondrial dynamics and function are hallmarks of these disorders. For example, in various embodiments, the disorder is selected from the group consisting of Alzheimer's disease, Parkinson's disease, amyotrophic lateral sclerosis, mood disorders, and schizophrenia. Some compounds described herein, or those identified by the in vitro methods of this disclosure, protect mitochondria in primary neurons from Aβ(1-42) toxicity, glutamate toxicity, increased oxidative stress, and the cellular environment of Alzheimer's disease. The compounds are therefore useful, according to various embodiments, in the treatment of Alzheimer's disease.

### Pharmaceutical Composition

The present disclosure also provides in another embodiment a pharmaceutical composition comprising a compound or pharmaceutically acceptable salt thereof as described herein in combination with a pharmaceutically acceptable carrier or excipient. Compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the compound or a pharmaceutically acceptable salt thereof.

For tablet compositions, the compound or a pharmaceutically acceptable salt thereof in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the compound or a pharmaceutically acceptable salt thereof is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compound or a pharmaceutically acceptable salt thereof in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the compound or a pharmaceutically acceptable salt thereof in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compound or a pharmaceutically acceptable salt thereof may also be administered in the form of suppositories for rectal administration. These compositions can be prepared by mixing the compound with a suitable non-irritating excipient which is solid at ordinary temperatures but liquid at the rectal temperature and will therefore melt in the rectum to release the compound. Exemplary excipients include cocoa butter and polyethylene glycols.

Compositions for parenteral administrations are administered in a sterile medium. Depending on the vehicle used and concentration the concentration of the compound or a pharmaceutically acceptable salt thereof in the formulation, the parenteral formulation can either be a suspension or a solution containing dissolved compound. Adjuvants such as local anesthetics, preservatives and buffering agents can also be added to parenteral compositions.

### EXAMPLES

General Procedures and Materials for Examples 1-7

Mouse lines. The Mt mouse line was generated at the ROSA26 locus in a C57BL/6J (The Jackson Laboratories) background using standard knockin methodology. The targeting vector contained a CAG promoter, a loxP-flanked STOP cassette (STOP codons with all 3 reading frames), and a mitochondrial targeted GFP2 (Mt-GFP) and nuclearly targeted tagBFP2 transgenes (nls-BFP) separated by T2A peptide sequences [37]. A WPRE at the 3′ end was included to enhance mRNA stability. The Nls-BFP signal was not utilized in this screen. A similarly engineered ROSA26 knock-in (Ai14 strain; 007908, The Jackson Laboratory), which contains a Cre-dependent cassette for the expression of cytosolic tdTomato (Cyto-tdTomato) was a kind gift from Damon Page (TSRI, Florida). Mt mice were used in the primary screen. Progeny of Mt mice crossed with Ai14 mice that carried both fluorophores were used in the rescreen.

For respiration experiments using isolated mitochondria, C57BL/6J mice were grouped to receive 25 mg/kg dyclonine-supplemented or standard water for 7 months starting at 2 months of age. The water supply was refreshed weekly. Mice were housed on a 12 h: 12 h light dark cycle with ad libitum access to food and water. All experiments were performed during the light part of the diurnal cycle. Housing, animal care and experimental procedures were consistent with the Guide for the Care and Use of Laboratory Animals and approved by the Institutional Animal Care and Use Committee of The Scripps Research Institute.

Virus. A replication deficient AAV9 virus was packaged with a custom designed plasmid containing a CaMKII promoter to confer neuron-specific expression followed by iCre and tdTomato sequences in tandem but separated by T2A peptide sequences. The expression of tdTomato was at very low levels and not utilized here.

Primary forebrain cell culture and maintenance. Forebrains of P0 Mt or Mt/Ai14 pups without olfactory bulbs and meninges were collected and placed in ice cold Ca²⁺ and Mg²⁺ free HBSS supplemented with 2 μg/ml gentamicin, 25 mM D-glucose, 1 mM pyruvate and 20 mM HEPES. Brain tissue was digested with 0.6 mg/ml papain at 37° C. for 20 min. Forebrains were washed with plating media [Neurobasal (Gibco), 5% FBS, 4 mM GlutaMAX (Gibco), 2 μg/ml gentamicin]. The tissue was gently triturated, allowed to settle and the supernatant was filtered through a 40 μm mesh-sized cell strainer. Cells were centrifuged at 420×g for 4 min and resuspended in plating media. 15,000 cells per well were plated with 2×10⁸ viral particles/ml onto black Greiner μClear (Cat #781946) or Ultra-Low Base Aurora with evaporation barrier (Cat #ABE201201B) 384-well plates (80 μl/well) pre-coated with poly-D-lysine for the primary screen and rescreen, respectively. Plating and media changes were performed using an automated Biomek FX^{P} liquid handling workstation. Four hr after plating, 75% of the plating media was replaced by feeding media [Neurobasal-A (Gibco), 4 mM GlutaMAX (Gibco), 2 μg/ml gentamicin, 2% B27 (Gibco)]. At DIV4, 50% of the media was exchanged with fresh feeding media supplemented with 16 μM 5-Fluoro-2′-deoxyuridine (FUdR) to prevent glial cell over-growth. At DIV8 and DIV12, additional 50% media changes were made without FUdR. Cells were maintained at 37° C. and 5% CO₂; plates were topped with Breathe-Easy adhesive membranes (Greiner plates) to minimize evaporation.

Compounds and compound transfer. The Spectrum Collection (Microsource) consists of structurally diverse compounds covering a large chemical space with 1600 compounds currently marketed or in clinical trials, 300 natural products and their derivatives and 500 bioactive compounds not yet in clinical trials. For the primary screen and rescreen, 100 nl of 10 mM compounds (in DMSO) was pintooled into each well on DIV13 using the Biomek FX^{P} robot, resulting in 12.5 μM final concentration of compound and 0.125% final DMSO concentration per well. 100 nl of FCCP (in DMSO) was pintooled some wells at a 12.5 μM final concentration as a negative control. The primary screen and rescreen were performed with 4 and 10 replicate plates, respectively. Repurchased compounds and peroxide (Luperox, tert-butyl hydroperoxide) for all other experiments were dissolved in DMSO at the desired concentrations and pintooled similarly, keeping the final 0.125% DMSO concentrations in each well.

Glutamate (MSG, monosodium glutamate) was dissolved in 50-50% DMSO-water and pintooled as described above. Glutamate and peroxide stocks were made fresh. FCCP was protected from light and kept at −20° C. as a 10 mM stock solution dissolved in DMSO to avoid freeze-thaw cycles. Compound plates for all assays were kept at −80° C. and thawed just before use.

Amyloid-(3 peptide treatment. Aβ(1-42) and Aβ(1-40) peptide fragments were prepared fresh as described previously [5]. Briefly, HFIP-treated peptide (BACHEM) was solubilized in water-free DMSO to 2.2 mM and sonicated in a bath-sonicator for 30 s. Cold feeding media without B27 was added to achieve a concentration of 125 μM of peptide. Peptide was incubated at 4° C. for 24 h and centrifuged at 12,000×g for 10 min. The formation of oligomers was confirmed by native polyacrylamide gel electrophoresis. After determining the concentration of the oligomeric peptide in the supernatant, the oligomers were added to the mouse primary neuronal cultures at the desired concentration on DIV13 and incubated for 2 days.

Mitochondrial dyes. To assay the electrochemical potential across the inner mitochondrial membrane of cultured cells, we used the cell-permeant dye tetramethylrhodamine methyl ester (TMRM). For co-localization studies with Mt-GFP, TMRM was used at 20 nM on DIV14 and incubated for 30 min prior to imaging. For the orthogonal screen of compounds, TMRM was added to primary neurons on DIV13 at 10 nM, one hr prior to pintooling selected compounds, and incubated for 24 hr. Cells were washed 2× with pre-warmed fresh feeding media without B27 before measurements with a plate reader.

The cell permeable dye MitoTracker® Orange (ThermoFisher) was used to label mitochondria in live primary neurons according to the manufacturer's protocol. Briefly, neurons at DIV14 were stained with 50 nM MitoTracker Orange dissolved in fresh feeding media and incubated for 30 min. The cells were washed 2× with fresh feeding media before imaging.

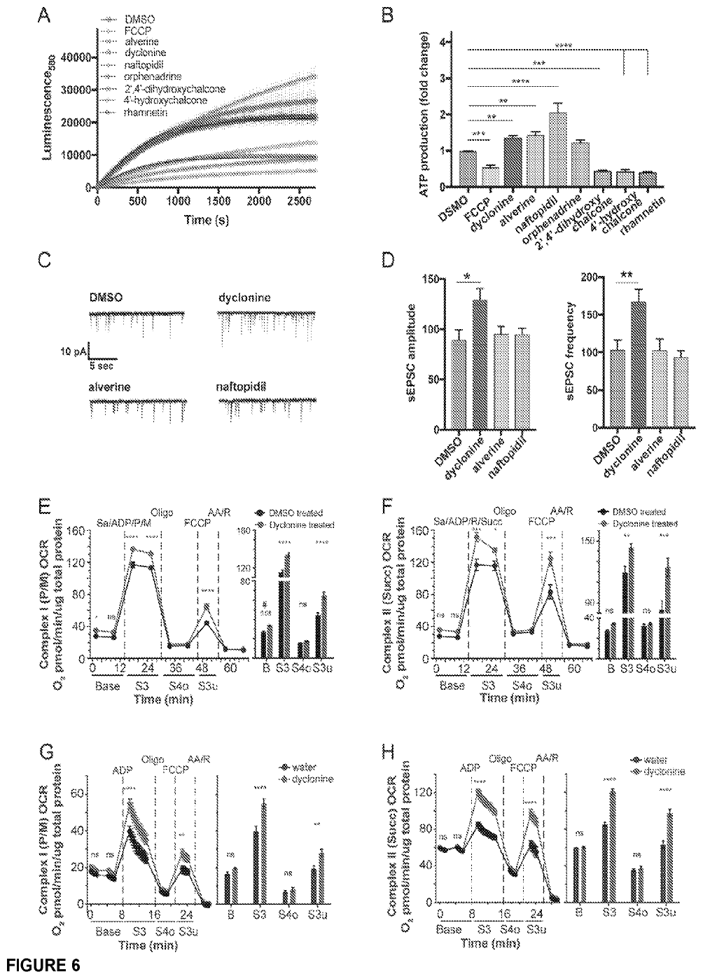

Isolation of mitochondria. For the ATP synthesis experiments of FIG. 6, forebrains from P0 pups were dissected as described above. 2×10⁷ cells were harvested for high-purity mitochondrial isolation using the Qiagen QProteome kit. The mitochondrial pellet was resuspended in 500 μl mitochondria storage buffer (provided by kit) and kept on ice until further use. Mitochondrial protein content was determined to be within 0.3-0.4 μg/1.11.

Whole brains were harvested from 2 mice/group of 9-month old C57BL/6J mice receiving 25 mg/kg dyclonine-supplemented or standard water for 7 months. Mitochondria were isolated as described in [38], method ‘A’ with modifications. Briefly, fresh brain tissue was minced and homogenized in a 40-ml Dounce homogenizer with cold isolation buffer (10 mM Tris, 1 mM EDTA, 110 g/L sucrose, pH 7.4), performing all subsequent steps on ice. The supernatant from two consecutive 5-min centrifugations at 1,300×g were combined and centrifuged for 10 min at 21,000×g. The harvested pellet was resuspended in 15% Percoll and layered above a 23% over 40% Percoll gradient. The gradient was centrifuged at 30,700×g for 15 min in a SW41Ti rotor and the mitochondrial fraction (a band at the 23%/40% Percoll interface) was aspirated. Mitochondria were washed and pelleted at 16,900×g for 10 min and the pellet was precipitated by adding BSA and centrifuging at 6,700×g for 10 min. The final mitochondrial pellet was resuspended in cold MAS-mitochondrial buffer without BSA (70 mM sucrose, 220 mM mannitol, 10 mM KH₂PO₄, 5 mM MgCl₂, 2 mM HEPES and 1 mM EGTA, pH=7.2 adjusted using KOH). The yield of mitochondrial protein was determined by BCA assay.

Measurement of ATP production. The total amount of ATP produced by cultured primary neurons was measured by CellTiter-Glo Luminescent Cell Viability Assay (Promega). A recombinant luciferase enzyme catalyzed the mono-oxygenation of luciferin while hydrolyzing ATP produced by the cells and emitting light. We followed the manufacturer's protocol with primary neurons on DIV14.

ATP production of isolated forebrain mitochondria of P0 pups was measured by ATP Determination Kit (Molecular Probes). Briefly, isolated mitochondria were diluted 20× in ATP assay buffer (225 mM sucrose, 44 mM KH₂PO₄, 12.5 mM Mg acetate, 6 mM EDTA) and incubated with 10 μM of compounds for 20 min Mitochondria were further diluted 10× with the standard reaction solution (provided by the kit; includes luciferin-luciferase reagents) containing 10 μM of compounds, 100 μM pyruvate, 5 μM palmitoyl-L-carnitine, 1 mM α-ketoglutarate and 100 μM malate in addition. The reactions were initiated by the addition of 1 mM ADP (deprived of ATP contamination by adding 3 U/ml hexokinase to 10 mM of ADP in 50 mM Tris-HCl, 20 mM MgCl₂ and 22 mM glucose, incubating the solution at 37° C. for 1 hr and heat inactivating the hexokinase at 100° C. for 3 min). Luminescence was recorded every 15-30 sec for 1 hr at 580±40 nm.

Respiration of isolated mitochondria. The oxygen consumption rate (OCR) of permeabilized neurons and isolated brain mitochondria (from 9-month old mice) was measured by the XF96 Extracellular Flux analyzer (Seahorse Bioscience). For assays using cultured neurons, primary neurons were dissociated from the forebrains of P0 pups, seeded at 1.9×10⁴/well (PDL-coated XF96 plate), cultured to DIV13 and incubated with dyclonine or DMSO (0.1%) for 24 hours. Prior to the start of the OCR measurement, all but 30 μl of Neurobasal A/B27 culture medium was removed from each well. Cells were washed twice with pre-warmed MAS-BSA assay medium (4 mg/ml fatty-acid free BSA, pH=7.2) and incubated in 180 μl at 37° C. for 5 min. Following the cartridge calibration, cells were loaded into the XF96 Extracellular Flux analyzer and further equilibrated for 10 min with two cycles of 3 min mixing and 2 min resting prior to the first measurement of basal respiration. OCR were measured at 37° C. under basal conditions followed by the sequential injection of pre-warmed 10× mitochondrial substrates/ADP (20 μl), oligomycin (22 μl), FCCP (24 μl), and rotenone/antimycin A (26 μl). The final concentrations of injected compounds were as follows: 10 mM mitochondrial substrates (pyruvate/malate or succinate), 1 mM ADP, 1.5 μM oligomycin, 3 μM FCCP, 2 μM rotenone/antimycin A. Saponin (25 μg/ml) was co-injected with substrate/ADP to permeabilize the cells and stimulate ADP-dependent respiration. Two baseline measurements were obtained prior to any injection and 2 response measurements were collected after each injection except for after FCCP addition which consisted of 1 measurement (9 total measurements in each assay). Each measurement cycle consisted of 2 min mixing, 2 min waiting, and 3 min data acquisition. This protocol allowed for sequential assessment of basal cell respiration, maximal mitochondrial respiratory capacity (State 3 with substrate/ADP), proton leak (State 4ₒ with oligomycin), uncoupled maximal respiration (State 3_{U} with FCCP) and non-mitochondrial respiration with the Complex I/Complex III inhibitors, rotenone/antimycin A. Cells were washed twice with MAS buffer to remove residue BSA contained in the assay medium, lysed in 50 μl lysis buffer (25 mM HEPES, 1 mM EGTA, 1 mM EDTA, 0.1% SDS, 1% NP-40, 1× protease and phosphatase inhibitor, pH 7.0 adjusted with NaOH) and total protein was determined by BCA assay. Oxygen consumption was normalized to total protein content as pmol O₂/min/ug total protein.

For OCR measurements of isolated brain mitochondria, the mitochondria were diluted to 3 μg/20 μl in cold MAS-BSA buffer+substrate (pyruvate/malate or succinate, to maintain healthy state of mitochondria) and plated onto 96-well PDL-coated plates (20 μl/well). Wells without mitochondria were used for background correction. The plate was centrifuged at 2000×g for 20 min at 4° C. to attach the mitochondria. After centrifugation, an additional 160 μl of MAS-BSA buffer+substrate was added to the wells. Mitochondria were checked under the microscope to ensure a homogenous mitochondrial monolayer in each well and incubated at 37° C. for 10 min. The plates were further equilibrated for 8 min by two cycles of 1 min mixing and 3 min rest prior to the measurement of basal respiration. Two baseline measurements were obtained prior to any injection, and one response measurement was obtained after each injection followed by additional 30 sec mixing. The final concentration of compounds after injections were as follows: 10 mM pyruvate/malate or succinate, 1 mM ADP, 2 μM oligomycin, 4 μM FCCP, 2 μM rotenone/antimycin A. Each measurement cycle consisted of 30 sec mixing, and 3 min data acquisition except for the measurement after ADP injection which lasted 6 min to observe the transition from State 3 to State 4 due to the depletion of ADP in the microchamber. The additional mixing step after each measurement facilitated the sensor returning to ambient O₂ concentration. State 3 respiration parameters driven by mitochondrial Complex I substrates (pyruvate/malate) were measured first, while Complex II-driven respiration (succinate) was measured by inhibiting Complex I with rotenone (2 μM). OCR was calculated by the Seahorse XF96 software package. OCR data measured from isolated brain mitochondria is displayed in the “point-to-point mode” showing a series of OCR rates across each measurement period.

Spontaneous excitatory postsynaptic currents in hippocampal neurons. Primary neurons were isolated from the hippocampi of C57BL/6J mice at embryonic stage 16 (E16), plated onto PDL-coated cover-slips and cultured in complete medium (Neurobasal 1×, 2% B27, 2 mM GlutaMAX™ and 2 μg/ml gentamicin) until DIV8 to allow the development of extensive synaptic networks. The cultures were treated with selected compounds or 0.1% DMSO for 24 hr before measuring spontaneous excitatory postsynaptic currents (sEPSCs). Whole-cell patch-clamp recordings were performed using an Axon Multiclamp 700b amplifier, 1440A Digidata digitizer and data acquisition using pClamp software (Axon Instruments, Foster City, Calif.). The neurons were first recorded in a current clamp mode to monitor cell health. Only neurons with a resting membrane potential of less than −40 mV were used for further analysis. sEPSC recordings were made at 50 kHz and subsequently filtered at 5 kHz. The membrane potential was held at −70 mV during the recording. The extracellular bath solution for the recordings contained 135 mM NaCl, 10 mM glucose, 3 mM CaCl₂, 2 mM KCl, 2 mM MgCl₂, and 5 mM HEPES, adjusted pH to 7.4 with NaOH, and to 300-315 mOsm with sucrose. Patch-pipettes were pulled from borosilicate glass with a micropipette puller (Sutter Instrument Co.) and filled with intracellular solution containing 100 mM K-gluconate, 1.7 mM KCl, 0.6 mM EGTA, 5 mM MgCl2, 10 mM Hepes, 4 mM ATP, and 0.1 mM GTP, adjusted pH to 7.2 with NaOH and to 300-315 mOsm with sucrose. Once a neuron was patched, its seal was monitored, and if the transient resistance was less than 100 mOhm, the recording was not used for analysis. All experiments were performed at room temperature. The frequency and amplitude of sEPSCs were analyzed using the template match search (pClamp) and measured as a percentage of baseline level, calculated from the average of a 5 min baseline recording. The values of the amplitude and frequency of EPSCs for each recording was reported as an average from a 5 min recording period.

Live-Dead assay. After cultured primary forebrain neurons to DIV15, media was removed from the wells and propidium iodide (PI) dissolved in Hank's Balanced Salt Solution was added at 100 μM. The cells were incubated for 2 min followed by the addition of equivalent volume of Hank's Balanced Salt Solution containing the cell permeable, fluorescein diacetate (FDA) yielding final concentrations of 12 μM for FDA and 50 μM for PI. Cells were imaged 5 min later.

Imaging. Images were captured using a GE Incell 6000 system (60× objective, 0.95 NA, 16-bit images) equipped with environmental control (37° C., 5% CO₂). Mt-GFP (green channel) was imaged by confocal imaging (3 slices, Δz=0.7 μm, aperture=1.0 AU, λ_{exc}=488 nm, λₑₘ=515-535 nm). Cyto-tdTomato (red channel) and MitoTracker Orange (red channel) were imaged using confocal mode (3 slices, Δz=0.7 μm, aperture=1.0 AU, λ_{exc}=561 nm, λₑₘ=569-641 nm, MitoTracker Orange) or with widefield mode (Cyto-tdTomato). TMRM imaged for the co-localization experiments with Mt-GFP was imaged using the confocal mode (3 slices, z=40.7 aperture=1.0, λ_{exc}=488, λₑₘ=569-641 nm). For the live-dead assay, primary neurons were imaged with a 20× (widefield mode, NA 0.45) objective using λ_{exc}=561 nm and λₑₘ=569-641 nm for the propidium iodide stained dead cells (red channel), and λ_{exc}=488 nm and λₑₘ=515-535 nm for the fluorescein diacetate stained live cells (green channel). 10 fields were collected from each well, and 4 wells were imaged/condition in the live-dead assay. For all other experiments, 4 fields/well were imaged unless otherwise stated.

A CLARIOstar (BMG LABTECH) microplate reader was used to measure fluorescence of TMRM and luminescence produced from the ATP. Fluorescence was measured by applying 540±18 nm excitation and 585±20 nm emission filters in the orbital averaging well scan mode, 17 flashes/well. Luminescence was measured through an emission filter of 571±80 nm, detecting light from the whole well.

Automated data analysis pipeline. Most experiments utilized only Mt-GFP; some utilized both Mt-GFP and Cyto-tdTomato. The Z-projected images were preprocessed using a custom Fiji macro. For preprocessing of Mt-GFP images: background was subtracted (rolling ball, radius=3), images were median filtered (radius=1) and somatic mitochondria were removed. For removal of the somatic mitochondria, they were first segmented and then a somatic mitochondrial mask overlaid on the image. Steps for somatic mitochondria segmentation were: taking square root of the image (equalization of the too bright and too dim somatic mitochondria), minimum filtering (kernel=20) to erode small structures, using gaussian filter (sigma=20) to ensure mitochondria in neurites were downscaled, Otsu thresholding, object filtering (5000-100000 pixel), and binary dilation (15 cycle) of segmented somatic mitochondria. The size or shape of the mitochondria in neurites did not change during this preprocessing. For preprocessing of Cyto-tdTomato images: applying tubeness filter [39] followed by the removal of the soma, using the somatic mitochondrial mask from the green channel. For further details see also [40].

Segmentation of the preprocessed images followed using GE Developer software. Images were equalized to the complete 16-bit dynamic range and segmented using object-based segmentation with kernel size of 3. Segmented objects were classified as axonal mitochondria (green channel, length: 0.5<Mtₐₓₒₙ<=1.4 μm, intensity: >5000, area: >0.25 μm², circularity: >0.6); dendritic mitochondria (green channel, Mt_{dend} length: >=2.4 inn, intensity: >5000); and neurites (red channel, intensity >5000, circularity <0.5). Count, length, area and circularity were measured for axonal and dendritic mitochondria, area was measured for neurites, and the measurements were aggregated using the median for mitochondrial area, circularity and length and a sum of the area for mitochondria (CA) and neurites.

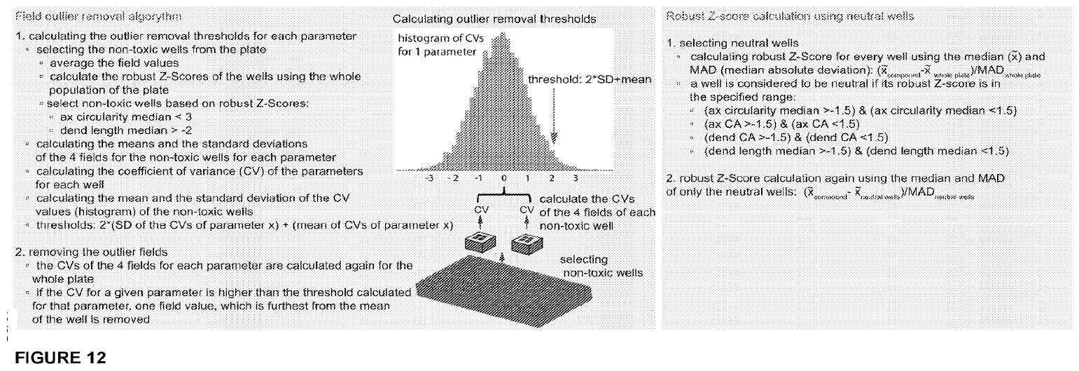

Aggregated field values were further processed by a custom Python script. Field values containing less than 500 mitochondria were removed, along with outliers, identified by a custom written machine learning algorithm (FIG. 12, left panel), and field values were averaged to well values. Robust Z-scores were calculated using neutral wells (FIG. 12, right panel) for the primary screen, and DMSO treated wells for the rescreen. Outliers were removed from the replicate plate well values and the replicate plates were averaged. Replicate plate wells containing outliers were identified by selecting those where the standard deviations of the replicates' robust Z-scores were larger than 3. In those cases, one value, which was furthest from the mean was removed. In the rescreen, where 10 replicate plates were used instead of 4, this algorithm was run 3 times, allowing the removal of maximum 3 values from the 10 replicates. Finally, hits were selected based on their robust Z-scores. For axonal mitochondrial content hits: axonal mitochondrial CA >2.5 & dendritic mitochondrial CA between −2 and 2. For dendritic mitochondrial content hits: CA >2.5 & axonal mitochondrial CA <2. For elongation hits: dendritic mitochondrial length median >2.5. For health hits: axonal mitochondrial circularity median <−2.5.

Structural clustering of hits. Morgan fingerprints of the rescreened hits were calculated using RDKit. A distance matrix using the dice similarities of the fingerprints were created, and clustering was performed by WPGMA (Weighted Pair Group Method with Arithmetic Mean) method.

Statistical Analysis. N=4 (from 1 experiment) and N=10 (from 3 independent experiments) replicate plates (1 well/compound/plate, 4 fields/well, n=1000-5000 mitochondria/field) were used for the primary screen and rescreen, respectively. Hits were picked based on robust Z-score cut-off of 2.5. Detailed data analysis can be found in the automated data analysis pipeline section. Compounds are significantly different from control if < or > than 2 robust Z-scores. Data of functional assays are from N=4 replicate plates (8 wells/compound/plate, 2 independent experiments). Statistical tests were performed using GraphPad Prism 7.0 (GraphPad Software, Inc.).

### Example 1: Developing a Multiplexed High-Throughput and High-Content Phenotypic Screen for Modulators of Neuronal Mitostasis Using Primary Neurons

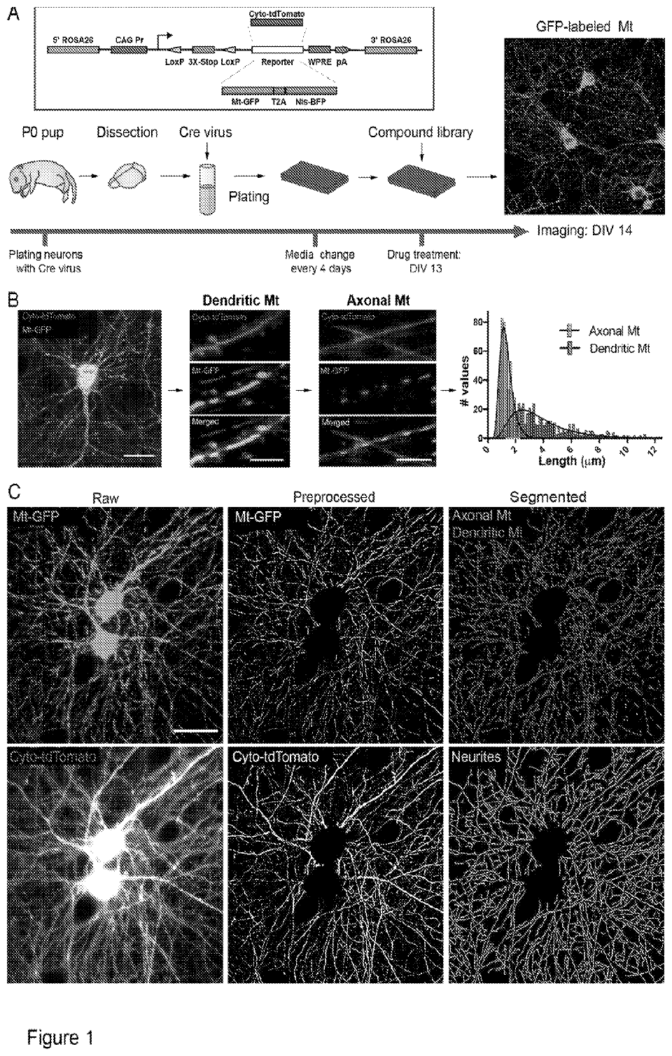

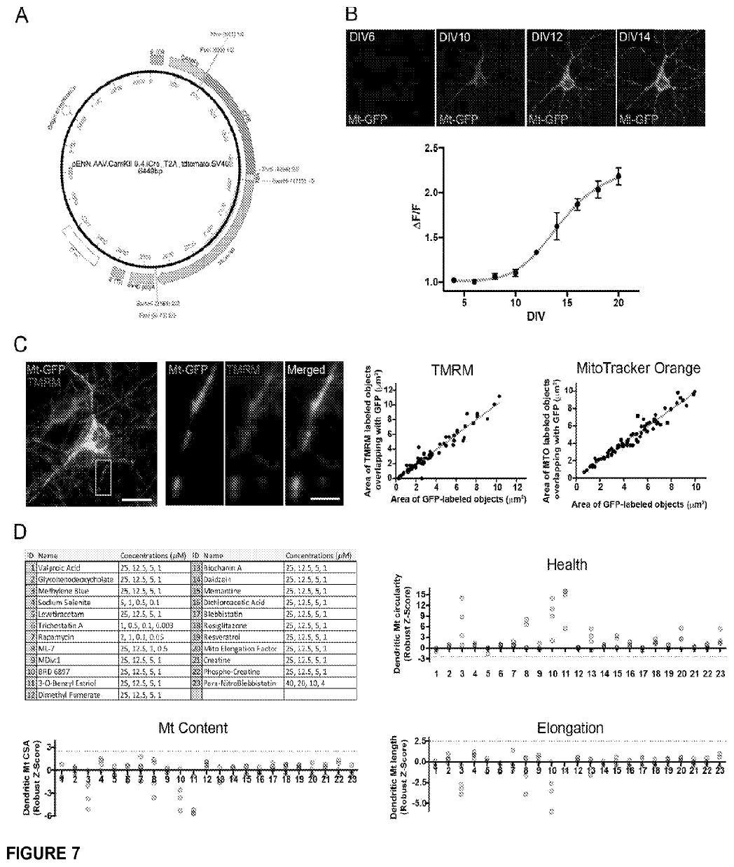

To simultaneously assay multiple processes of neuronal mitostasis in primary neurons, we generated a ROSA26 knock-in mouse line carrying a floxed tagGFP2 expression cassette with an N-terminal mitochondrial targeting sequence. This strain was generated in a C57Bl/6J background (FIG. 1A; referred to as Mt mice). Dissociated cells from the forebrains of P0 pups of Mt mice were mixed with replication deficient AAV9 carrying a CaMKII promoter driven iCre recombinase gene (FIG. 7A) and plated onto 384-well plates. Employing Cre-dependent, mitochondrial GFP expression offers experimenter control over the number of GFP-labeled mitochondria in each imaging field so they are not too dense nor too sparse. The floxed Mt expression cassette also included a nuclear-localized tagBFP2, which was used for the visualization of the neuronal nuclei. GFP labeled mitochondria (Mt-GFP; mitochondrial GFP) began to appear ˜DIV8 (8 days in vitro) and by DIV14 the signal was sufficiently strong to be imaged and analyzed (FIG. 7B). Compound treatment was performed 24 hr prior to imaging, on DIV13. Correct sub-cellular targeting of the mitochondrial GFP reporter was validated by co-staining the Mt-GFP with tetramethylrhodamine methyl ester (TMRM) and MitoTracker Orange (MTO), two well-characterized, cell-permeable fluorescent probes known to label mitochondria (FIG. 7C).

Mitochondria in neurons are morphologically heterogeneous. In the soma, they form an irregularly shaped interconnected network while in neurites they exist as rod-like structures of various lengths. We focused our efforts on discovering compounds that increase mitochondrial content, promote elongation, or induce healthier, less round mitochondria in the axons and/or dendrites.

We first classified Mt-GFP into three classes: somatic, axonal and dendritic mitochondria. The separation of axonal and dendritic mitochondria was achieved by their length differences. To measure the length differences between axonal and dendritic mitochondria, we used neurons from pups of Mt mice crossed with a ROSA26 knockin for expressing cytoplasmic tdTomato (Cyto-tdTomato, FIG. 1A), which provided a strong signal in the neurites. We then measured the length of ˜500 mitochondria in axons (axonal mitochondria) and ˜500 in dendrites (dendritic mitochondria) (FIG. 1B). We considered mitochondria to be axonal if located in a process that was thin with a presynaptic bouton and dendritic if located in a thick process populated with dendritic spines. By plotting the frequency distribution of the mitochondrial length values, we found that mitochondria with lengths between 0.5-1.4 μm are predominantly axonal, while mitochondria with lengths longer than 2.4 μm are predominantly dendritic, therefore we excluded the range 1.4-2.4 μm from the analysis of axonal and dendritic mitochondria. This resulted in eliminating 4.5% of axonal mitochondria (0.4-1.4 μm) and 3% of dendritic mitochondria (>2.4 μm). To identify somatic mitochondria, we took advantage of their tightly packed, bright and circular shape and analyzed them as one mass. Since extracting individual mitochondrial features from somatic mitochondria was not feasible, we excluded them from further analysis.

We considered the possibility that compounds might increase mitochondrial content by promoting neurite sprouting. This hypothesis envisions that the compounds might increase neurite sprouting with mitochondrial biogenesis being spurred to fill the new neurites with mitochondria. Alternatively, increased biogenesis of mitochondria might drive neurite sprouting to accommodate the additional organelles. It was also possible that compounds might increase the biogenesis-turnover balance without altering neurite sprouting, which would increase the density of the mitochondrial mass in the processes. To distinguish between these possibilities, the Cyto-TdTomato signal was used in our rescreen to probe for altered neurite area.

To collect high quality data with sufficient replication for the screen, we used automated confocal imaging and captured 4 z-stacks in each well across 4 replicate plates (FIG. 1C). We then preprocessed and segmented the raw images to identify dendritic and axonal mitochondria and measured their area, length and circularity. The morphological data of the mitochondria of every field were aggregated to a population sum (of individual mitochondrial area) or median (of the population of individual mitochondrial circularity and length) values.

### Example 2: Defining Image-Based Parameters for Mitochondrial Content, Elongation and Health

There are currently no small molecule probes reported to have a robust beneficial effect on mitostatic processes of primary neurons. As a first step, we selected and tested 23 compounds with reported effects on mitochondrial dynamics in other cell types, attempting to find positive controls for a screen for mitochondrial content, elongation or health in neurons (FIG. 7D). Unexpectedly, none of the tested compounds had marked effects on the measured morphological features of mitochondria in primary forebrain neurons. In contrast, toxic compounds causing mitochondrial loss, fragmentation and swelling were readily available [16]. We chose to use the uncoupling agent FCCP to perturb mitochondrial function and morphology to help identify and validate the image-based parameters chosen as measures of mitostatic processes. FCCP treatment induced dramatic mitochondrial loss and fragmentation (FIG. 2A).

To quantify the mitochondrial content in the images, we defined the cumulative area of a field (CA) as the sum of the areas occupied by individual mitochondria. FCCP treatment caused a large decrease in the number of dendritic mitochondria and their average area (FIG. 2B, left panel), and a ˜90% reduction in the average dendritic mitochondrial CA per well (FIG. 2B, right panel). The area distribution pattern for average axonal mitochondria did not change, but the number of mitochondria classified as axonal increased (FIG. 2C, left panel). This resulted in an increase of axonal mitochondrial CA/well upon FCCP treatment (FIG. 2C, right panel). The average sums of the dendritic and axonal mitochondrial CAs upon DMSO or FCCP treatment altogether indicates a neuron-wide mitochondrial loss due to FCCP treatment (CA_{Dend+Ax,DMSO}=1292+404=1696 μm², CA_{Dend+Ax,FCCP}=123+629=754 μm²). Although the observed increase in axonal mitochondrial CA (225 μm²) due to FCCP treatment probably arises from excessive fragmentation of dendritic mitochondria yielding short objects that become classified as axonal mitochondria, this increase accounts for only ˜20% of the decrease of the dendritic CA. For this reason, we chose the parameter of dendritic mitochondrial CA >2.5 robust Z-scores for identifying compounds that increase mitochondrial content.

To identify compounds that elongate mitochondria we focused on the feature of dendritic length. FCCP treatment of neurons produced shorter dendritic mitochondria (FIG. 2D, left panel). Since individual dendritic mitochondrial length measures did not follow a normal distribution, we chose the median of the population within a field to obtain field values. Moreover, the median is less sensitive to outliers than the mean of the population, thus more reliable for a screen. FCCP treatment produced a significant decrease in the median of dendritic mitochondrial length (FIG. 2D, right panel). Thus, we chose median dendritic mitochondrial length as the measure of elongation and defined hits that increase mitochondrial length as those that increase this parameter by more than 2.5 robust Z-scores.

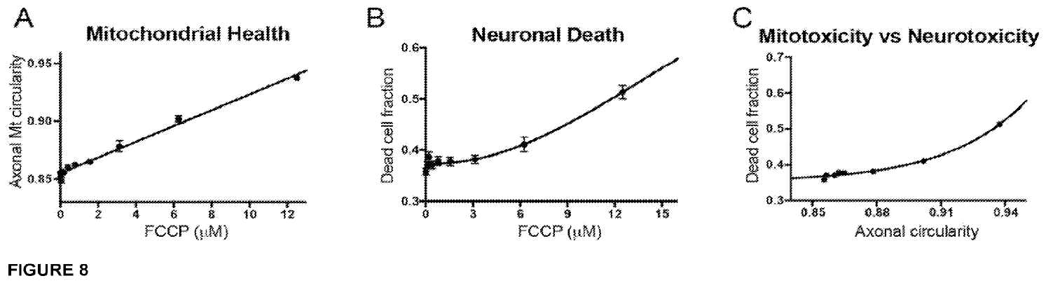

We did not observe a significant decrease in average axonal mitochondrial area with FCCP treatment, but the axonal mitochondria became more rounded compared to their usual oblong shape. This increase in roundness of the axonal class can be measured by circularity (4πarea/perimeter²), a measure that correlates well with mito- and neurotoxicity (FIG. 8). The ratio of highly circular mitochondria increased in the axonal mitochondrial population with FCCP treatment (FIG. 2E, left panel), and this was reflected in the average of axonal circularity median as well (FIG. 2E, right panel). Highly circular mitochondria are well documented to occur in many pathological conditions [17, 18]. Therefore, we chose a decrease in the median of axonal mitochondrial circularity by >2.5 robust Z-score for identifying a third group of compounds—enhancers of mitochondrial health. In summary, FCCP treatment of neurons produced marked mitochondrial loss, dendritic mitochondrial fragmentation and more circular axonal mitochondria (FIG. 2), as also described by others [16]. Given this relationship, we concluded that small molecules that increase mitochondrial content or elongation, and those that decrease circularity, offer a starting point in identifying probes that improve the mitochondrial system in neurons.

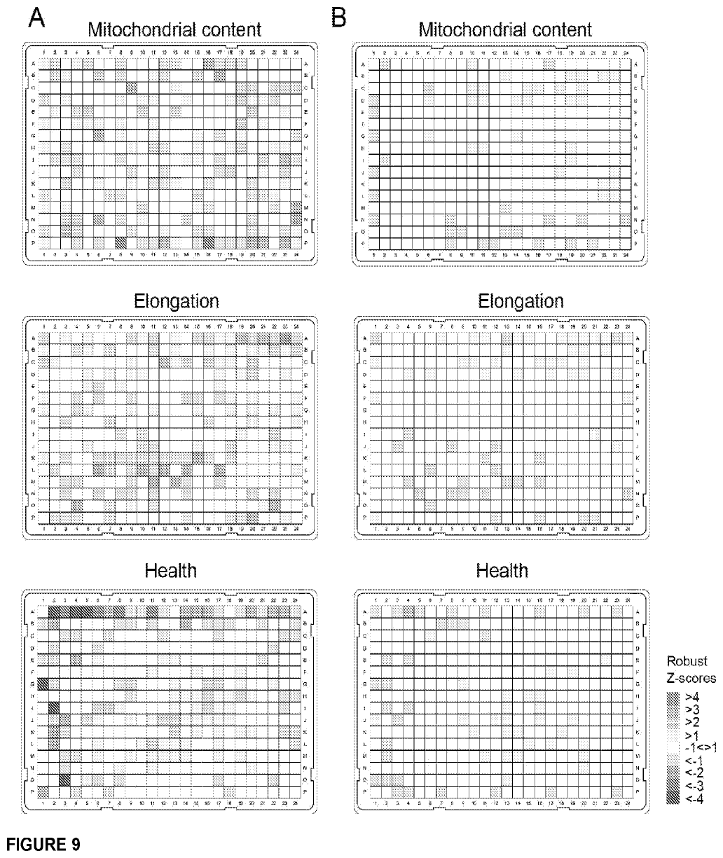

We constructed an automated data analysis pipeline for our screen, where the input values were the medians (circularity, length) or sums (CA) of the individual mitochondrial measures of the fields. This data analysis pipeline is described in detail in Materials and Methods. A control experiment performed using this image and data analysis pipeline and vehicle treated plates failed to identify any false positives in any of our hit categories (FIG. 9).

### Example 3: Small Molecule Screen and Rescreen for Modulators of Neuronal Mitostasis

We screened a chemical library consisting of 2400 compounds (Spectrum Collection from MicroSource) for modulators of neuronal mitostasis using the screening platform and data analysis pipeline described above. We used FCCP as a negative control and neutral wells from the plate interior for calculating robust Z-scores.

In the primary screen, we selected 149 putative hits (6.2%) that met our threshold for modulating mitochondrial content (>2.5 robust Z-scores), elongation (>2.5 robust Z-scores) and/or health (<−2.5 robust Z-scores), (FIG. 3A-C). Many compounds in the library proved to be neurotoxic, causing mitochondrial loss, fragmentation and increased circularity similar to FCCP treatment. The putative hits were rescreened using 42 DMSO treated wells scattered randomly across the plate for the calculation of robust Z-scores. As expected, the population of wells treated with putative hits yielded mitochondrial features that were shifted from the population of wells treated with DMSO (FIG. 3D-F), indicating a significant hit enrichment. Close examination of selected hit compounds on mitochondrial features (FIG. 3G-K) revealed that they had an opposite effect of FCCP treatment as predicted. Elongation hits led to an increased median of dendritic mitochondrial length (FIGS. 3G and K). Mitochondrial content enhancers increased the count and area occupied (CA) by dendritic mitochondria (FIGS. 3H and K). Health enhancers decreased the number of rounded axonal mitochondria and increased the number of elongated ones, thereby decreasing the median of axonal mitochondrial circularity (FIGS. 3I and J).

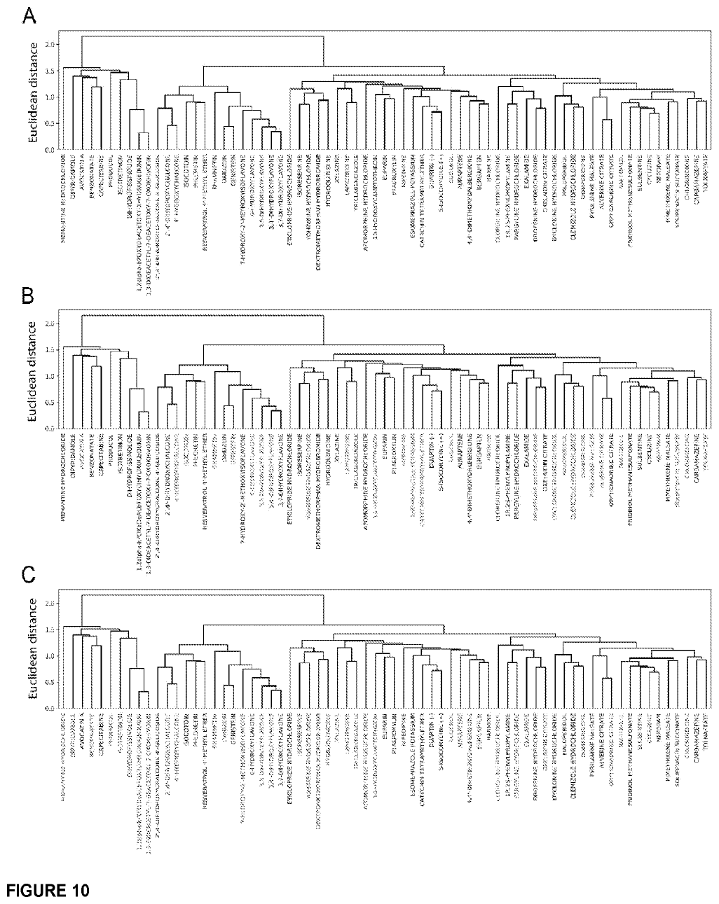

We confirmed 67 of the initial 149 compounds in the rescreen (45%; FIG. 3L) using 10 replicate plates. Notably, some molecules both present in the Spectrum Collection and in the candidate molecule screen were confirmed in the re-screen as weak hits (daidzein, memantine and the 4′-methyl ether derivative of resveratrol). Of the confirmed hits, 32 increased elongation, 45 increased mitochondrial content and 33 increased mitochondria health (FIG. 3L). Thirty-five of the compounds altered more than one mitochondrial parameter. Analysis of the compounds based on structural fingerprint similarities revealed that those modulating the three hit categories fell into multiple structural clusters (FIG. 10), and none of the hit categories enrich structurally similar compounds. This suggests that multiple and diverse mechanisms of action can lead to similar effects on aspects of mitochondrial dynamics. The classification itself revealed 3 primary clusters. Two of the clusters contained compounds that are structurally distinct. Most members from the third cluster are from the flavonoid family (flavones, isoflavones, chalcones), implying common targets.

### Example 4: Mitostasis Processes and Function Dissected with Small Molecule Probes

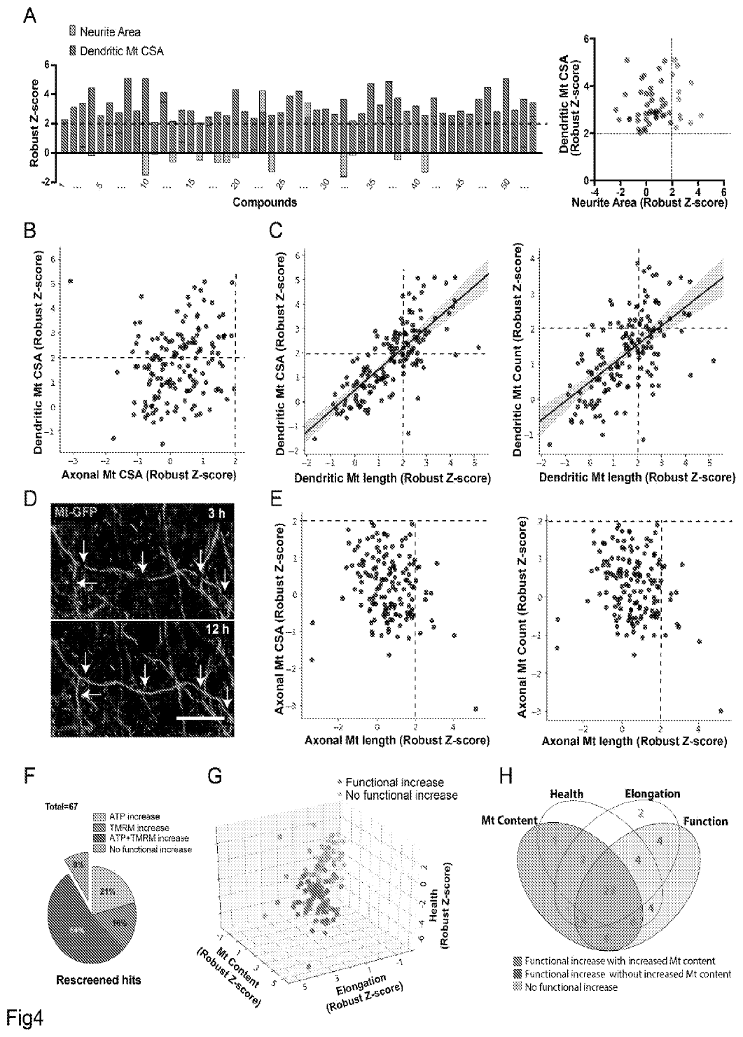

To distinguish compounds that increase mitochondrial content with or without neurite sprouting, we measured the total area covered by neurites across the fields imaged for each well in the rescreen (FIGS. 1C and 4A left panel). Eleven of the 53 compounds which increased mitochondrial content significantly (>2 robust Z-scores) also increased neurite area by more than 2 robust Z-scores (Table 1), although there was no correlation between dendritic mitochondrial CA increase and neurite area across this population (FIG. 4A, right panel). The lack of correlation between these two parameters surprisingly suggests that they are not coupled: mechanisms that increase mitochondrial content are not triggered in response to increased sprouting, nor is increased neurite sprouting triggered by events that enhance mitochondrial content. A complete understanding of how neurons provide a sufficient supply of mitochondria to distal neurites has not been achieved, although biogenesis in the soma and anterograde transport along with de novo synthesis in the neurites offer partial explanations [1, 19].

TABLE 1
The effect of MnMs on neurite sprouting. The table shows the
dendritic Mt CA and neurite area robust Z-scores of the mitochondrial
content enhancers. The ID numbers of the compounds correspond
to the ID numbers on FIG. 4A. Mt = mitochondrial.

| | | Dendritic Mt | |
|---|---|---|---|
| ID | Compound Name | CA | Neurite Area |
| 1 | 10-HYDROXYCAMPTOTHECIN | 2.23 | 2.29 |
| 2 | 1R,2S-PHENYLPROPYLAMINE | 3.14 | 1.25 |
| 3 | 2′,4′-DIHYDROXYCHALCONE | 3.39 | 0.42 |
| 4 | 2′,4′-DIHYDROXYCHALCONE 4′- | 4.44 | −0.18 |
| | GLUCOSIDE | | |
| 5 | 2′,4-DIHYDROXYCHALCONE | 2.57 | 0.01 |
| 6 | 3,5-DIHYDROXYFLAVONE | 3.43 | 1.20 |
| 7 | 3,7-DIHYDROXYFLAVONE | 2.78 | 1.37 |
| 8 | 4′-HYDROXYCHALCONE | 5.11 | 2.21 |
| 9 | 6-HYDROXYFLAVONE | 2.88 | 0.69 |
| 10 | ALVERINE CITRATE | 5.09 | −1.50 |
| 11 | APOMORPHINE HYDROCHLORIDE | 2.10 | −0.07 |
| 12 | AVOCATIN A | 4.14 | 3.48 |
| 13 | BUDESONIDE | 2.16 | −0.61 |
| 14 | CANAGLIFLOZIN | 2.93 | 0.74 |
| 15 | CAPECITABINE | 2.88 | 0.58 |
| 16 | CARBARIL | 2.03 | −0.48 |
| 17 | CATECHIN TETRAMETHYLETHER | 2.44 | 1.87 |
| 18 | CHOLEST-5-EN-3-ONE | 2.78 | −0.65 |
| 19 | CLEMIZOLE HYDROCHLORIDE | 2.54 | −0.66 |
| 20 | CLORGILINE HYDROCHLORIDE | 4.33 | −0.36 |
| 21 | CYCLIZINE | 2.82 | −0.03 |
| 22 | DEXTROMETHORPHAN | 2.38 | 0.21 |
| | HYDROBROMIDE | | |
| 23 | DIHYDROFISSINOLIDE | 2.77 | 4.24 |
| 24 | DIPYRIDAMOLE | 2.60 | −1.31 |
| 25 | DOMPERIDONE | 2.76 | 2.59 |
| 26 | DYCLONINE HYDROCHLORIDE | 3.89 | 0.33 |
| 27 | EUPARIN | 4.22 | 1.17 |
| 28 | EXALAMIDE | 2.43 | 3.42 |
| 29 | GENISTEIN | 2.93 | 0.34 |
| 30 | HALOPERIDOL | 2.99 | −2.37 |
| 31 | HALOTHANE | 2.60 | 2.65 |
| 32 | HARMINE | 3.65 | −1.63 |
| 33 | HYDROQUINIDINE | 2.19 | −0.15 |
| 34 | ISOTRETINON | 2.69 | 0.77 |
| 35 | LAMOTRIGINE | 4.75 | 1.00 |
| 36 | MEMANTINE HYDROCHLORIDE | 3.25 | 1.45 |
| 37 | NAFTOPIDIL | 4.86 | 2.41 |
| 38 | NEFOPAM | 3.74 | −0.44 |
| 39 | NIFEDIPINE | 2.85 | 0.22 |
| 40 | ORPHENADRINE CITRATE | 3.21 | 0.08 |
| 41 | OXELAIDIN CITRATE | 2.59 | −1.31 |
| 42 | PARGYLINE HYDROCHLORIDE | 3.75 | 2.51 |
| 43 | PHLORETIN | 2.73 | 1.23 |
| 44 | PIMETHIXENE MALEATE | 2.57 | −2.10 |
| 45 | PRIDINOL METHANESULFONATE | 2.86 | 1.80 |
| 46 | PYRILAMINE MALEATE | 2.65 | 0.78 |
| 47 | RESVERATROL 4′-METHYL ETHER | 3.68 | 0.02 |
| 48 | RHAMNETIN | 4.48 | 2.04 |
| 49 | S-ISOCORYDINE (+) | 2.82 | 0.78 |
| 50 | SOLIFENACIN SUCCINATE | 5.06 | 1.42 |
| 51 | TOLNAFTATE | 2.93 | 1.01 |
| 52 | XYLAZINE | 3.66 | 0.40 |
| 53 | YOHIMBINE HYDROCHLORIDE | 3.44 | 2.50 |

Our rescreen data using compounds that modulate neuronal mitostasis along with neutral compounds offered a unique opportunity to explore the relationship between several mitochondrial properties in neurons. Plotting dendritic mitochondrial CA as a function of axonal mitochondrial CA showed that none of the 53 compounds that increased dendritic mitochondrial CA significantly (>2 robust Z-scores) increased axonal mitochondrial CA (FIG. 4B, Pearson's r=0.22). Given the large number of compounds with effects on dendritic mitochondrial CA, it seems unlikely that this results from the simple failure to find a compound that alters both. Rather, the result is more consistent with the possibility that increases in mitochondrial content can occur solely in dendrites, and/or that axonal mitochondrial content is more tightly regulated and is harder to perturb with small molecules. Nevertheless, compound-induced dendritic mitochondrial content seems to be uncoupled from axonal mitochondrial content. However, we did observe a correlation between dendritic mitochondrial CA and dendritic mitochondrial length as expected (FIG. 4C, Pearson's r=0.76). Moreover, dendritic mitochondrial count and dendritic mitochondrial length also correlated positively (FIG. 4C, Pearson's r=0.6), indicating that compounds that increase mitochondrial content often do so by increasing both count and length.

In order to explore the possible connection between mitochondrial content and elongation, we collected time-lapse images of primary neurons for 12 hr after treatment with content and elongation hits along with untreated controls. As reported by others [20], we observed fast moving short mitochondria that continuously underwent fusion and fission events or nearly stationary longer mitochondria which did not. However, in neurons treated with compounds, the stationary long mitochondria sometimes fused together by apparent growth towards each other (FIG. 4D). This observation explains the correlation between dendritic mitochondrial content and elongation. Compounds that increased axonal mitochondrial length did not significantly influence axonal mitochondrial CA or count (FIG. 4E).

### Example 5: MnMs Alter Mitochondrial Function

We tested the 149 rescreened compounds for effects on mitochondrial function by measuring two parameters, the inner mitochondrial membrane (IMM) potential and ATP generation. We used the well characterized rhodamine derivative, TMRM, to measure the IMM potential and a commercial kit for measuring ATP generation. Strikingly, 61 of the 67 confirmed hits (91%) also increased ATP production, TMRM signal or both significantly (>2 robust Z-scores), showing that the vast majority of MnMs also increase mitochondrial function (FIG. 4F). This indicates a tight link between mitochondrial dynamics and function.

Next, we probed the correlation between individual mitochondrial morphological features and function using the rescreened 149 compounds. Interestingly, this revealed only weak correlations between the individual mitochondrial features (area, length, count, CA and circularity) and function, with Pearson correlations (r) ranging from ˜0.4-0.5. This included the three hit parameters of mitochondrial content, elongation and health: dendritic mitochondrial CA (r_{ATP}=0.5, r_{TMRM}=0.47), dendritic mitochondrial length (r_{ATP}=0.41, r_{TMRM}=0.53), and axonal mitochondrial circularity (r_{ATP}=−0.44, r_{TMRM}=−0.41). However, plotting the rescreened compounds in three-dimensional space defined by the three hit parameters and separately marking whether they increase function or not revealed that compounds boosting mitochondrial function form a distinct cluster from those that do not (FIG. 4G). This indicates that increased mitochondrial function is generally accompanied by a combination of phenotypic changes that can be described by mitochondrial content, length, and health.

The functional increase of mitochondria induced by 61 different MnMs can occur by 1) an increase in mitochondrial mass present in the neurons (mitochondrial content), 2) no change in mass but an increase in the efficiency of individual mitochondria, and 3) a combination of 1) and 2). To help distinguish between these possibilities, we constructed a four-way Venn diagram of the compounds that increased function, content, health or length (FIG. 4H). Most of the compounds that increased mitochondrial function (n=61) also increased mitochondrial content (n=49), revealing an expected relationship between increased mitochondrial mass and measured function. However, the screen also identified compounds that only increased ATP production and/or membrane potential change without increasing content (n=12), suggesting an operative increase of output efficiency of the existing mitochondria. All these compounds improved health and/or induced elongation while increasing function. The Venn diagram further supports that all 3 parameters drive functional increases with a similar contribution (number of compounds increasing the hit parameter and function/number of compounds increasing hit parameter): biogenesis 92% (49/53), health 93% (39/42), elongation 90% (45/50).

### Example 6: Modulators of Mitochondrial Dynamics Protect Mitochondria Against Insults Associated with Neurodegenerative Disorders

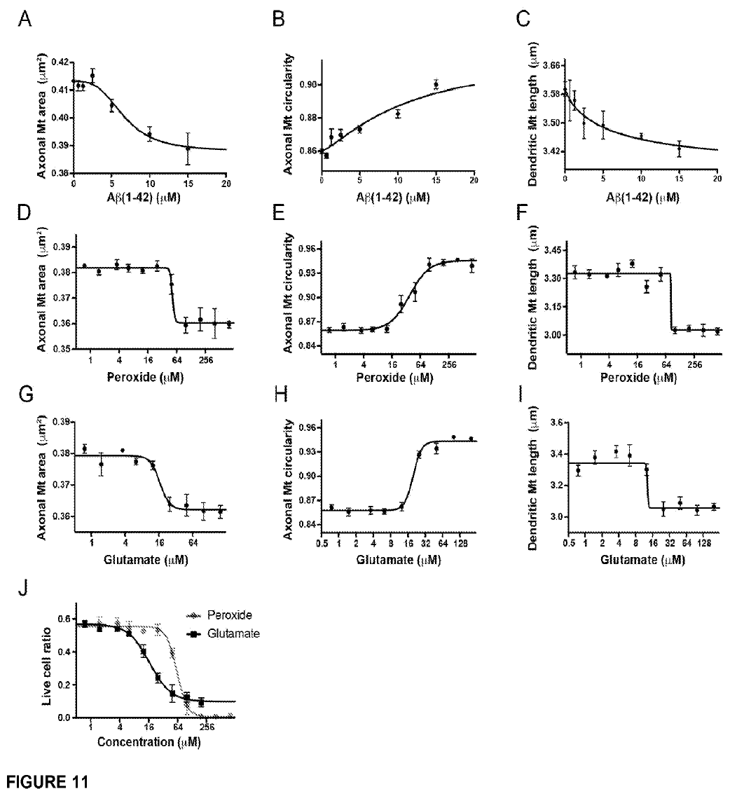

Altered mitochondrial dynamics and function have been identified to be part of the neuropathology found in neuropsychiatric disorders along with increased oxidative stress and glutamate toxicity [5-10]. We challenged primary neurons with increasing concentrations of toxic Aβ(1-42) oligomers, peroxide (tert-butyl hydroperoxide) and excess glutamate in order to obtain a deeper understanding of the mitochondrial phenotypes that emerge (FIG. 11). We observed severe fragmentation of both axonal and dendritic mitochondria with all three treatments, identified by a decrease of axonal mitochondrial area, a decrease in dendritic mitochondrial length, and an increase of axonal mitochondrial circularity. Treatment of neurons with 10 μM Aβ(1-42) oligomers, 75 μM peroxide or 25 μM glutamate for 48 h produced reproducible and sufficient effect sizes for our assays of mitotoxicity while keeping a significant fraction of neurons alive.

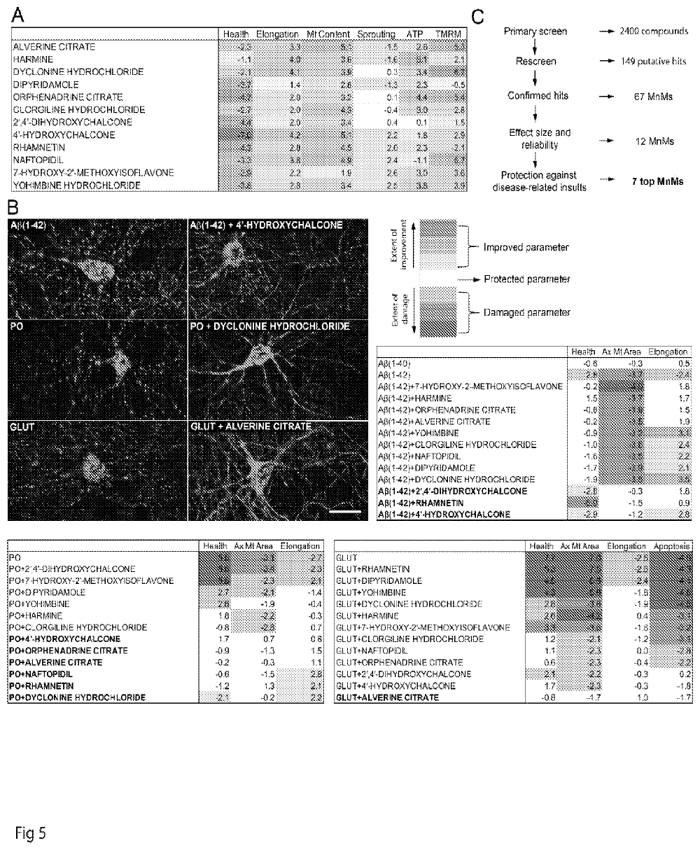

We then treated neuronal cultures with the same three toxic substances, in the absence or in the presence of the 12 most reliable MnMs chosen by effect sizes for mitochondrial function and mitostasis parameters, to determine whether the modulators offered protection against the toxic agents assayed 48 hr later. The MnMs by themselves had robust effects on mitochondrial health, elongation or content (FIG. 5A). Five of the MnMs induced neuronal sprouting (increased neurite area) and nearly all increased ATP production and/or mitochondrial membrane potential. Most of the MnMs exhibited relatively high potency and dose dependent effects on processes of mitostasis and neurite sprouting, with EC₅₀ values in the low to sub-micromolar range (Table 2).

TABLE 2
EC₅₀ values of selected MnMs for processes of mitostasis and neurite sprouting.
Neurons were treated with 0-25 μM of the compounds on DIV13 and imaged
24 h later. Normalized data (13 plates, 1 well/concentration/plate, 3 independent
experiments) were fit with a variable slope sigmoidal dose response curve, yielding
EC₅₀ values (μM). Compounds without saturation (no sat.), with no dose
dependency (no dd.) or without a significant effect (—) on processes
of mitostasis or neurite sprouting are also indicated.

| | Health | Elongation | Mt Content | Sprouting |
|---|---|---|---|---|
| | (Ax Mt | (Dend Mt | (Dend Mt | (Neurite |
| Compound | Circularity) | length) | CA) | Area) |
| ALVERINE CITRATE | 0.3 | 2.9 | 0.2 | — |
| HARMINE | — | 1.7 | no dd. | — |
| DYCLONINE | 0.2 | 1.5 | 0.2 | — |
| HYDROCHLORIDE | | | | |
| DIPYRIDAMOLE | no sat. | — | no dd. | — |
| ORPHENADRINE | 0.4 | 0.5 | 0.2 | — |
| CITRATE | | | | |
| CLORGILINE | 0.3 | 5 | 1 | — |
| HYDROCHLORIDE | | | | |
| 2′,4′- | 6.7 | no dd. | no dd. | — |
| DIHYDROXYCHALCONE | | | | |
| 4′-HYDROXYCHALCONE | 5.8 | 2.3 | 4.8 | 0.9 |
| RHAMNETIN | 2.7 | 0.2 | 0.2 | 0.3 |
| NAFTOPIDIL | 0.2 | no dd. | 0.1 | 0.5 |
| 7-HYDROXY-2′- | 0.2 | no dd. | — | no dd. |
| METHOXYISOFLAVONE | | | | |
| YOHIMBINE | 1.3 | 0.2 | 0.01 | 1.7 |
| HYDROCHLORIDE | | | | |

We categorized the effects of MnMs in the presence of the toxic substances into 3 groups: (1) full protection: all three parameters measuring fragmentation (axonal mitochondrial area, dendritic mitochondrial length, axonal mitochondrial health) were normalized to within control values or improved, (2) partial protection: at least one of the three parameters was within control values or improved, and (3) no protection: none of the parameters were within control values or improved (FIG. 5B). The compounds exhibited different protective power against the three insults, suggestive of different mechanism of action (FIG. 5B). Rhamnetin, 4′-hydroxychalcone and 2′,4′-dihydroxychalcone conferred complete protection from Aβ(1-42) induced mitochondrial fragmentation. The nine other compounds provided only partial protection, failing to protect against the decreased area of individual axonal mitochondria caused by the toxic peptide. As a control, we treated the cultures with the less-toxic peptide, oligomeric Aβ(1-40). This oligomeric peptide failed to cause mitochondrial fragmentation under the conditions used.

Mitochondrial fragmentation caused by treatment with peroxide (PO) was fully protected by 6 compounds: 4′-hydroxychalcone, orphenadrine, alverine, naftopidil, rhamnetin and dyclonine; and partially protected by 4 others. Glutamate (GLUT) induced mitochondrial fragmentation was fully protected by one compound, alverine. We also measured the live:dead cell ratio in the presence of toxic concentrations of glutamate and the selected MnMs and found that alverine, along with 4′-hydroxychalcone and 2′,4′-dihydroxychalcone, offered protection. In summary, 7 of the MnMs offered complete protection against Aβ(1-42) oligomer, peroxide or glutamate induced mitochondrial fragmentation in addition to their robust and reliable improvement of mitostatic processes using wild type neurons (FIG. 5C).

### Example 7: Dyclonine Enhances Mitochondrial Function Directly, Potentiates Basal Synaptic Activity, and Increases the Respiration of Brain Mitochondria in Treated Mice

To determine whether select compounds alter mitochondrial function directly, isolated forebrain mitochondria of P0 mouse pups were treated with the compounds and ATP production was measured using a luciferase-based kinetic assay (FIG. 6A, B). We observed a significant direct effect on mitochondria for 6 from the top 7 MnMs that offered complete protection against cell-based insults. Dyclonine, alverine and naftopidil significantly increased ATP production, while rhamnetin, 4′-hydroxychalcone and 2′,4′-dihydroxychalcone surprisingly decreased it within the 45-min measurement period. After 24 hr of treatment with the latter compounds, ATP production in neurons showed no significant change (with the 2 chalcones) or increase (rhamnetin, FIG. 5A), suggesting that the direct inhibition of ATP production by mitochondria may have been normalized via longer-term compensatory mechanisms. Dyclonine and alverine increased ATP production of mitochondria in primary neurons after 24 hr of incubation as well.

Primary hippocampal neurons were treated for 24 hr with the MnMs that increased mitochondrial function and sEPSCs were measured using patch-clamp techniques. Dyclonine was the only compound of the three that directly increased mitochondrial ATP production and significantly altered sEPSCs, increasing both frequency and amplitude (FIG. 6C, D). Thus, increased spontaneous activity is not a universal feature of compounds that increase mitochondrial ATP synthesis.

We selected dyclonine for subsequent tests, first to measure the rate of respiration in cultured neurons treated with dyclonine and second, to measure respiration in mitochondria isolated from mice treated chronically with dyclonine. The latter experiment was designed to test whether dyclonine, as a representative MnM, alters mitochondrial function in an in vivo environment. We measured the absolute oxygen consumption rate at baseline and in response to excess ADP/substrate (State3/3u) as normalized to protein content in primary neurons exposed to dyclonine for 24 hr. We observed a significant increase of State 3 and 3u respiration stimulated by either Complex I or Complex II substrates in mitochondria from the treated cultured neurons (FIG. 6E, F). A similar respiratory rate increase was observed in mitochondria isolated from the whole brains of mice treated chronically for 7 months with dyclonine laced into their water supply (FIG. 6G, H). The latter results demonstrate that dyclonine increases the capacity of substrate oxidation of mitochondria in vivo when administered chronically.

### General Procedures and Materials for Examples 8-12

Primary neuronal cultures in 384-well microplates for high-throughput screening. Primary neurons were prepared from the forebrains of C57BL/6J or 3×TG (Oddo et al., 2003) mice at P0. Briefly, the brains were dissected in cold buffer (1×HBSS w/o Ca²⁺ and Mg²⁺, 25 mM glucose, 20 mM HEPES, 10 mM pyruvate, 2 μg/ml gentamicin) and the meninges removed. Cells were dissociated by incubation with papain (0.015%) for 20 m at 37° C. followed by 5 cycles of trituration and seeded at 1.5×10⁴ cells/well into black-wall, clear bottom, 384-well poly-D-lysine coated microtiter plates (Greiner) and cultured in Neurobasal medium supplemented with 5% BSA, 2 mM GlutaMAX™ and 2 μg/ml gentamicin. For quality control, we checked the health of the dissociated primary neurons by measuring cell viability using automated cell counter (Invitrogen) according to manufacturer's manual. We only plated primary neurons exceeding 90% viability. The medium was refreshed with Neurobasal A complete medium (1× Neurobasal A, supplemented with 2% B27, 2 mM GlutaMAX™, 2 μg/ml gentamicin) 4-6 h after plating when the cells were firmly attached, and half of the medium was refreshed every 2-3 days until DIV13. Non-neuronal cell division was arrested at DIV4 by the addition of FUdR (8.1 μM 5-Fluoro-2′-deoxyuridine and 20.4 μM Uridine). Plates were covered with Breathe-Easy® sealing membrane (Sigma-Aldrich) to minimize uneven evaporation across each plate in the extended cultures and to permit gas exchange and limit cross-contamination.

Mitochondrial membrane potential (Δψₘ) and luminescence-based ATP assays. The assay to measure Δψₘ was performed using the general procedures described in Perry et al. (2011). Changes of ΔΨₘ in each well were monitored using the mitochondrial-specific, fluorescent dye TMRM (tetramethyl rhodamine, methyl ester; Molecular Probes) and measured using a CLARIOstar plate reader. Our assay development experiments established that 10 nM TMRM provided a non-quenching concentration and a linear assay. This concentration was used throughout our experiments. Compounds known to decrease ΔΨₘ such as FCCP decreased TMRM fluorescence at 4 h or 24 h incubation, and were classed as toxic compounds from the screen, while those that increased ΔΨₘ as shown with increased TMRM fluorescence were identified as potential hits. Neurons cultured to DIV13 in 384-well plates were loaded with 10 nM TMRM for 90 m before pin-tooling 100 nl compounds (final concentration, 12.5 μM). After 4 or 24 h of incubation, the wells were quickly washed twice with pre-warmed Neurobasal medium to remove free TMRM and the intracellular fluorescence measured by the plate reader (excitation 535±15 nm, emission 590±25 nm). Wells in columns 1 and 24 were not loaded with TMRM; these served to measure background signal which was subtracted from the wells to which TMRM was added. To measure the total ATP levels in neurons we utilized the CellTiter-Glo® luminescence-based ATP assay (Promega). DIV13 primary neurons were cultured for 24 h with either DMSO carrier or compound treatment, followed by removing 55 μl culture medium from each well. Plates were maintained at room temperature for 25 m, and 25 μl of CellTiter-Glo® Reagent with the substrate luciferin was then added to each well (Promega). Plates were covered with aluminum foil and shaken vigorously on an orbital shaker for 10 m at 200 rpm and maintained at room temperature for an additional 2 m in dark before being read with the plate reader set to the luminescence mode. Dose response experiments were performed by pre-diluting the compounds and pintooling such that DMSO concentration was always kept constant (0.125%).

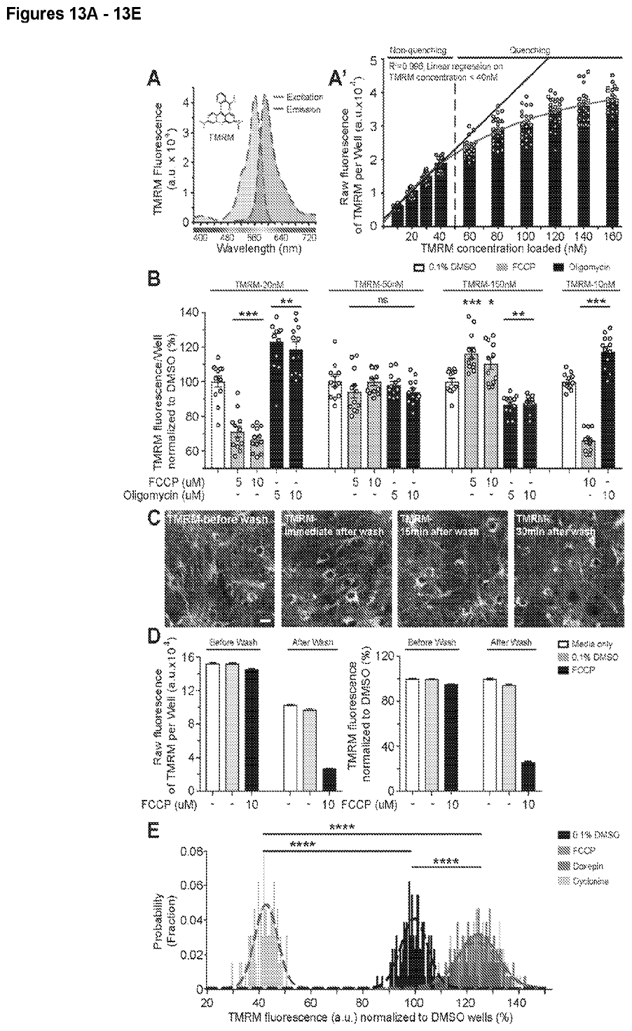

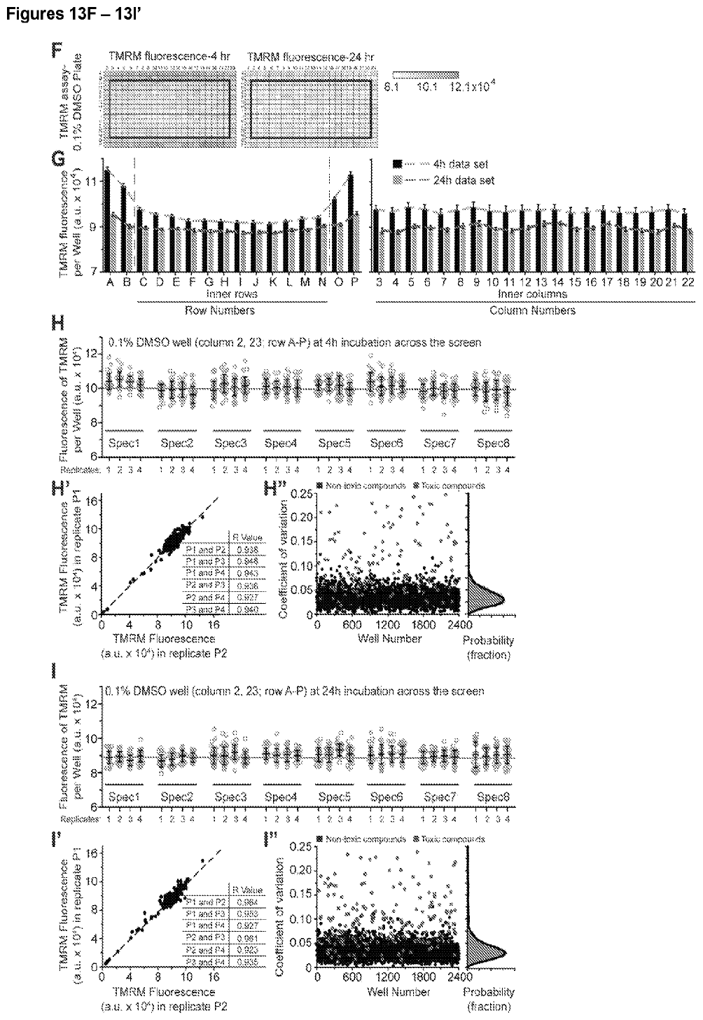

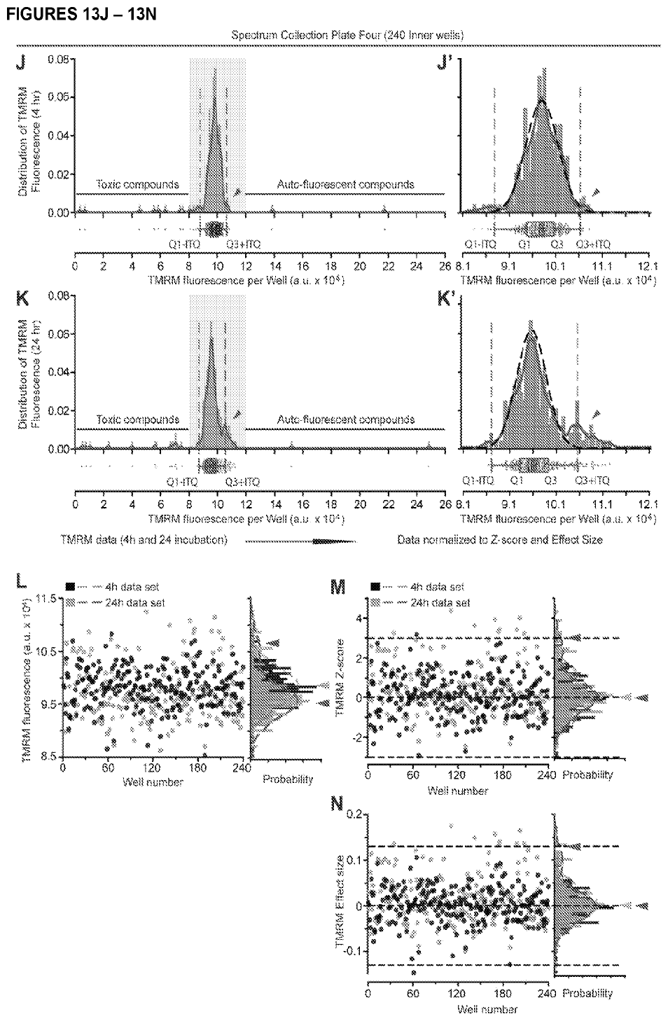

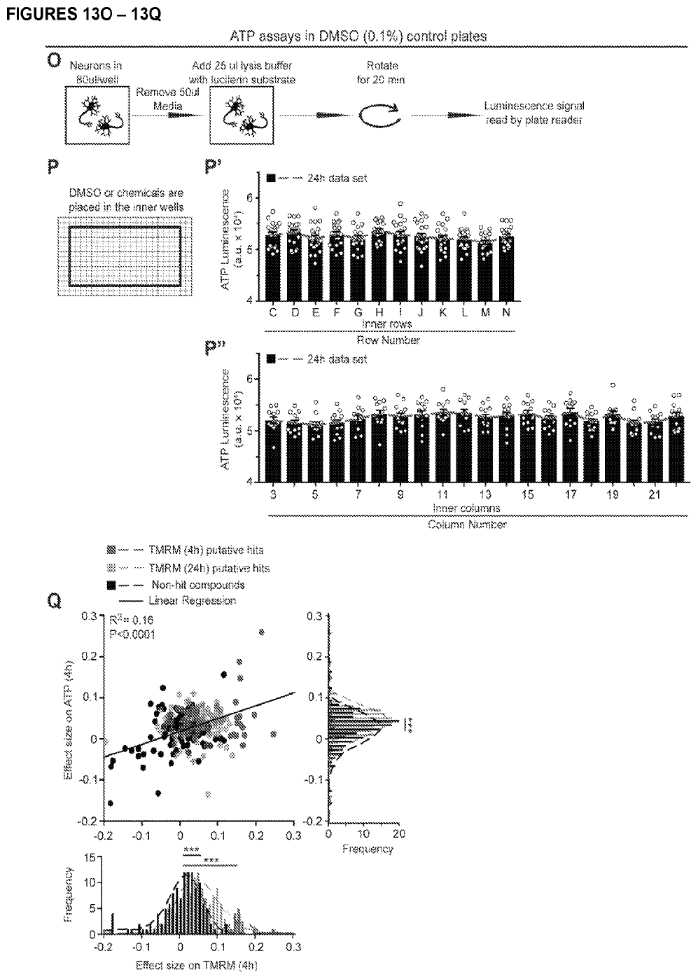

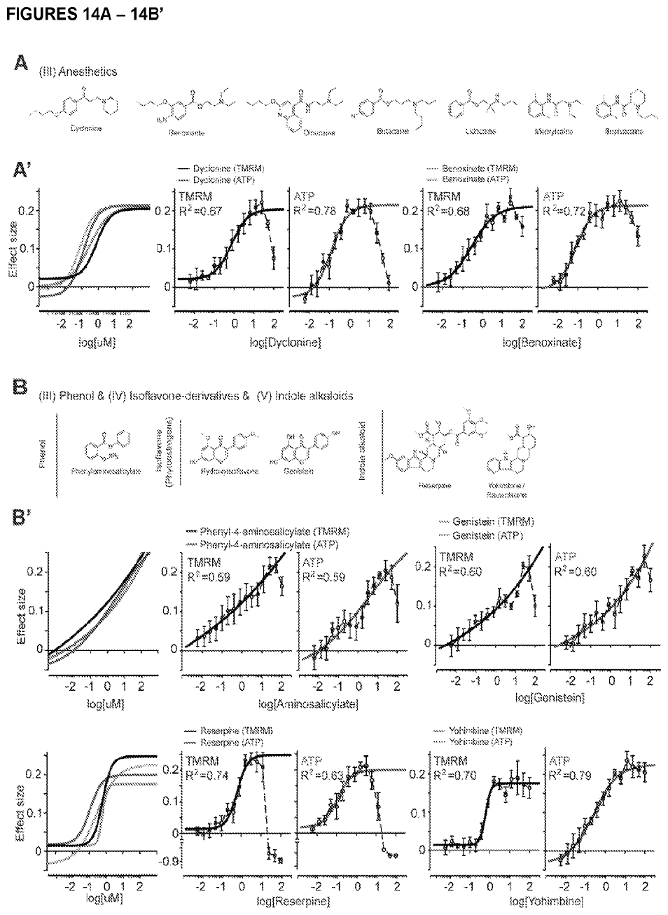

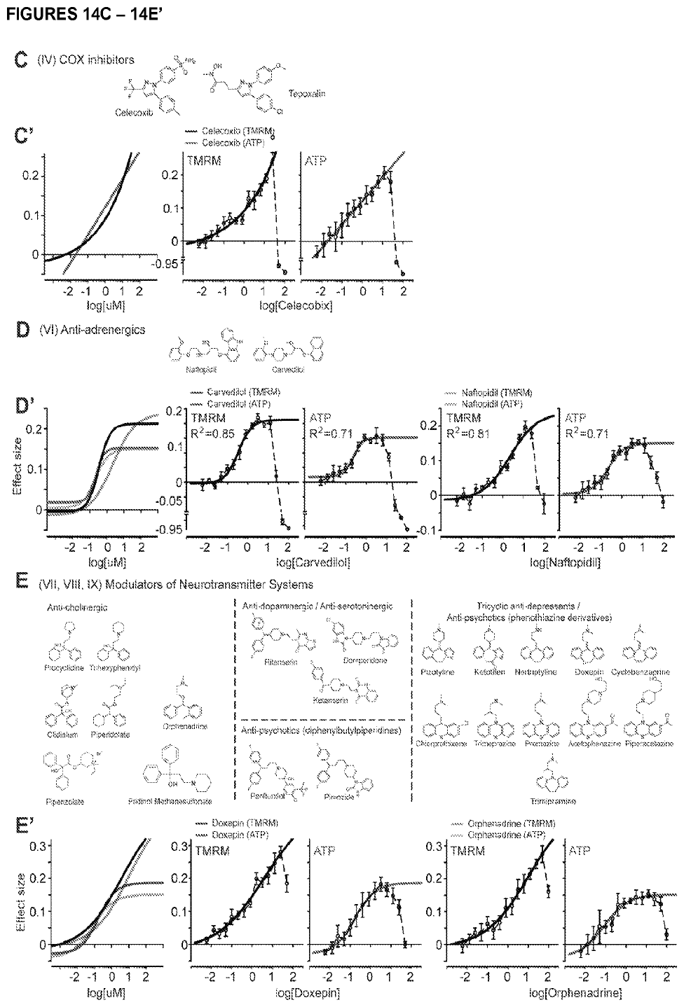

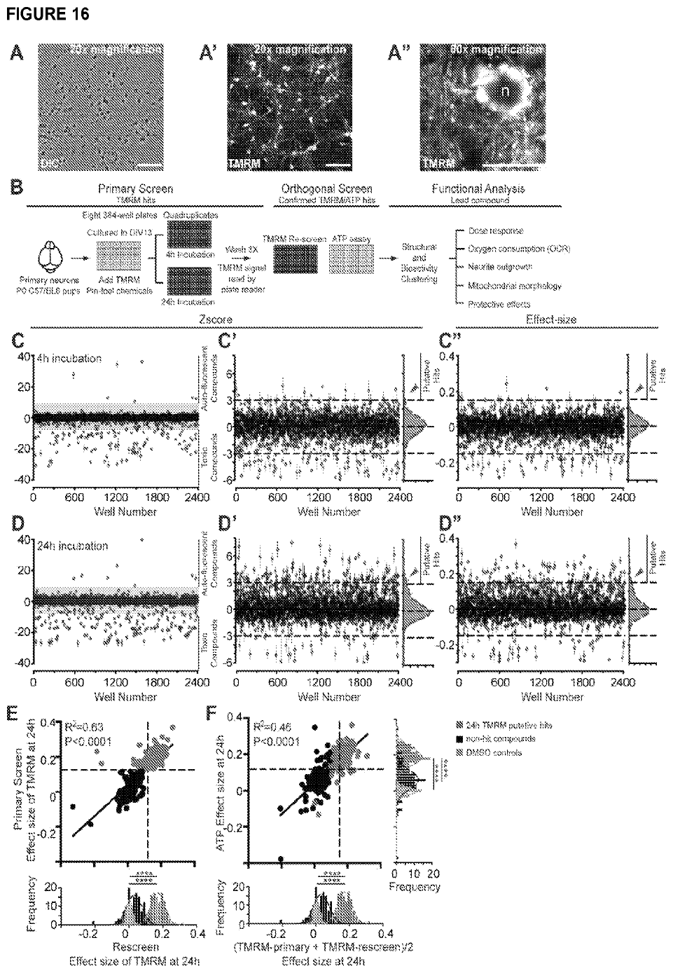

Compound library and hit picking rules. We screened 2400 compounds from the MicroSource Spectrum library. This library includes 1600 compounds from the US and International Drug Collections, compounds that have shown biological potential in peer-reviewed publications but have not been developed as drugs, and some nature products derived from commercial sources. FIGS. 13A-13E provide details of the assays used from which hit picking rules were made. In essence, the average fluorescence for each compound in the TMRM assay was determined and compared to the average fluorescence for compounds in the population on each assay plate to produce a Z-score. The Z-scores for each individual compound assayed across the four replicate plates was then averaged. Because of significant well location effects (outer rows) on each assay plate (FIG. 13G), we selected primary hits as enhancing well fluorescence above the μₚₗₐₜₑ by 3σₚₗₐₜₑ for inner row wells (rows C-N, columns 3-22), and by 2.5σₚₗₐₜₑ for outer row wells (rows A, B, O, P; columns 3-22) (FIG. 14C-D). Hits for the orthogonal ATP assay were selected in a similar way as elevating ATP levels by >3 Z-scores compared to within-plate DMSO controls (FIG. 16F, FIG. 13P). All orthogonal assays employed inner wells of the 384-well plates.

Structural clustering. Morgan fingerprints of the rescreened hits were calculated using RDKit. A distance matrix using the dice similarities of the fingerprints was created, and clustering was performed by WPGMA (Weighted Pair Group Method with Arithmetic Mean).

Mitochondrial morphology and neurite area measurements. Mitochondrial morphology and neurite area were quantified using methods described by Varkuti et al, 2020. Briefly, Cre-dependent mitochondrial targeted GFP2 (mito-GFP) ROSA26 knock-in mice were crossed with Ai14 (007908, The Jackson Laboratory) expressing cytosolic-tdTomato, and the forebrain of P0 pups were isolated and primary neurons were cultured as described above. AAV9 virus containing the iCRE sequence driven by the neuron specific CaMKII promoter was added to the cell culture at plating and 75% of the media replaced 4 h after plating. Imaging was performed 24 (DIV14) and 48 h (DIV15) after compound addition (GE IN Cell Analyzer 6000, 60× objective, 0.95 NA) in confocal mode for mito-GFP (green channel: 3 slices, Δz=0.7 aperture=1.0 AU, λ_{exc}=488 nm, λₑₘ=515-535 nm) and widefield mode for cytosolic-tdTomato (red channel: λ_{exc}=561 nm, λₑₘ=569-641 nm). Z-projected images of the green channel were preprocessed (background subtraction: rolling ball, radius=3; median filtering: radius=1) and somatic mitochondria were removed (see details in Varkuti et al, Science Advances 6 (2020) eaaw8702). Red channel images were also preprocessed (tubeness filter, soma removal). Preprocessed images were segmented by GE Developer software (object-based, kernel size: 3). Segmented objects of the green channel were classified as axonal (length: 0.5<Mtₐₓₒₙ<=1.4 inn, intensity: >5000, area: >0.25 μm², circularity: >0.6) or dendritic (length: >=2.4 inn, intensity: >5000) mitochondria, while those of the red channel were identified as neurites (intensity >5000, circularity <0.5). The length and circularity measurements for mitochondria were aggregated using the median and the area for neurites using the sum. After removing empty fields and outlier values from the fields, aggregated field values were averaged to well values (4 fields/well, 12 wells/compound). Robust Z-scores were calculated using DMSO-treated wells for each well ((median_{compound}−median_{DMSO})/median absolute deviation_{DMSO}), well values were averaged for each compound.

Isolation of brain mitochondria. C57BL/6J mice were randomly selected and grouped to receive either compound (5 mg/kg yohimbine)-supplemented or standard water for 7 months starting at 2 mo of age. The water supply was refreshed weekly. Mitochondria harvested from 2 brains per group were isolated in parallel by differential centrifugation as described by Sims and Anderson (2008), method “A” with some modifications. Briefly, the fresh brain tissue used for respiration experiments was minced and homogenized in a 40 ml Dounce homogenizer with cold isolation buffer (10 mM Tris, 1 mM EGTA, 110 g/L glucose, pH 7.4). All subsequent procedures were performed on ice. The supernatant from two consecutive 5 m centrifugations at 1,300×g were combined and spun for 10 m at 21,000×g. The harvested pellet was resuspended in 15% Percoll and layered above a 23% over 40% Percoll gradient. The gradient was centrifuged at 30,700×g for 15 m, and the mitochondrial fraction (a band at the 23%/40% Percoll interface) was aspirated. Mitochondria were washed and pelleted at 16,900×g for 10 m, and the loose pellet was precipitated by adding BSA and centrifuging at 6,700×g for 10 m. The final mitochondrial pellet was resuspended in cold MAS-mitochondrial buffer without BSA (70 mM sucrose, 220 mM mannitol, 10 mM KH₂PO₄, 5 mM MgCl₂, 2 mM HEPES and 1 mM EGTA, pH=7.2 adjusted using KOH) and the yield of mitochondrial protein was determined by BCA assay.

Measurement of mitochondrial O₂ consumption. Oxygen consumption rate (OCR) or extracellular acidification rate (ECAR) measurements from permeabilized neurons and isolated brain mitochondria were performed using the XF96 Extracellular Flux analyzer (Seahorse Bioscience). For assays using cultured neurons, primary neurons were dissociated from the forebrains of P0 pups, seeded at 1.9×10⁴/well (PDL-coated XF96 plate), cultured to DIV13 and incubated with selected compounds or DMSO (0.1%) for 24 h. Prior to the start of the OCR measurement, all but 30 μl of Neurobasal A/B27 culture medium was removed from each well. Cells were washed twice with pre-warmed MAS-BSA assay medium (70 mM sucrose, 220 mM mannitol, 10 mM KH₂PO₄, 5 mM MgCl2, 2 mM HEPES and 1 mM EGTA, 4 mg/ml fatty-acid free BSA, pH=7.2 adjusted using KOH) and incubated in 180 ul MAS-BSA medium at 37° C. in a CO₂-free incubator for 5 m. Following the cartridge calibration, cells were loaded into the XF96 Extracellular Flux analyzer and further equilibrated for 10 m with two cycles of 3 m mixing and 2 m rest prior to the first measurement of basal respiration. OCR and ECAR were measured at 37° C. under basal conditions followed by the sequential injection of pre-warmed 10× mitochondrial substrates/ADP (20 μl), oligomycin (22 μl), FCCP (24 μl), and rotenone/antimycin A (26 μl). The final concentrations of injected compounds were as follows: 10 mM mitochondrial substrates (pyruvate/malate or succinate), 1 mM ADP, 1.5 μM oligomycin, 3 μM FCCP, 2 μM rotenone/antimycin A. Saponin (25 μg/ml) was co-injected with substrate/ADP to permeabilize the cells and stimulate ADP-dependent respiration. Two baseline measurements were obtained prior to any injection and 2 response measurements were collected after each injection except for after FCCP addition which consisted of 1 measurement (9 total measurements in each assay). Each measurement cycle consists of 2 m mixing, 2 m waiting, and 3 m data acquisition. This protocol allowed for sequential assessment of basal cell respiration, maximal mitochondrial respiratory capacity (State 3 with substrate/ADP), proton leak (State 4ₒ with oligomycin), uncoupled maximal respiration (State 3_{U} with FCCP) and non-mitochondrial respiration with the Complex I/Complex III inhibitors, rotenone/antimycin A. Cells were washed twice with MAS buffer to remove residue BSA contained in the assay medium, lysed in 50 μl lysis buffer (25 mM HEPES, 1 mM EGTA, 1 mM EDTA, 0.1% SDS, 1% NP-40, 1× protease and phosphatase inhibitor, pH 7.0 adjusted with NaOH) and total protein was determined by BCA assay with a BSA standard curve. Oxygen consumption was normalized to total protein content as pmol O₂/min/ug total protein.

To assay mitochondrial respiration in mice receiving chronic treatment of selected compounds, brain mitochondria were isolated in parallel from C57BL/6J mice receiving water supplemented with or without 25 mg/kg dyclonine or 5 mg/kg yohimbine. The isolated mitochondria were resuspended in MAS buffer without BSA to determine the yield of mitochondrial protein and subsequently further diluted in cold MAS-BSA buffer+substrate (P/M or Succ) to 3 μg/20 ul. This mitochondrial suspension was plated onto PDL-coated 96-well assay plates at 20 μl/well, except for the wells in the four corners which were used for background correction. Substrate was initially included and maintained from this step on to help maintain a healthy state of mitochondria. The plate was centrifuged at 2000×g for 20 m at 4° C. to attach the mitochondria to the XF96 microplates. After centrifugation, 160 μl of MAS-BSA buffer+substrate was added to each well. The mitochondria were checked briefly under the microscope to ensure that the mitochondrial monolayer in each well was homogenous, and then cultured at 37° C. in a CO₂-free incubator for 10 m. The plates were loaded into the XF96 Extracellular Flux analyzer and further equilibrated for 8 m by two cycles of 1 m mixing and 3 m rest prior to the measurement of basal respiration. Two baseline measurements were obtained prior to any injection, and one response measurement was obtained after each injection followed by additional 30 s mixing. The final concentration of compounds after injections were as follows: 10 mM pyruvate/malate or succinate, 1 mM ADP, 2 μM oligomycin, 4 μM FCCP, 2 μM rotenone/antimycin A. Each measurement cycle consisted of 30 s mixing, and 3 m data acquisition except for the measurement after ADP injection which lasted 6 m. The extension in time for this measurement beyond 3 m was included to observe the transition from State 3 to State 4 due to the depletion of ADP in the microchamber. The additional mixing step after each measurement, though optional, facilitated the sensor returning to ambient 02 concentration. State 3 respiration parameters driven by mitochondrial Complex I substrates (pyruvate/malate) were measured first, while Complex II-driven respiration (succinate) was measured by inhibiting Complex I with rotenone (2 μM).

XF oxygen consumption rate (OCR) was calculated by Seahorse XF96 software package and the algorithm fully described in A. A. Gerencser et al., Anal Chem. 81 (2009) 6868-6878. OCR data measured from isolated brain mitochondria is displayed in the “point-to-point mode” showing a series of OCR rates across each measurement period. OCR data measured from permeabilized neurons is displayed in the “middle point” mode showing a single OCR rate for each measurement period, representing the average of the point-to-point rates.

Data analysis and statistics. All data are presented as mean±SEM unless otherwise noted. We used the “non-DMSO” control statistic approach for data normalization and hit selection in primary TMRM screen as described in FIG. 13. Experimental data containing only two groups were analyzed by Students t-test. A Pearson correlation coefficient was used to probe the relationship between the compounds' effect-size in TMRM fluorescence and ATP production. Data comparing multiple groups were analyzed by one-way ANOVA followed by Dunnett's post-hoc test. The data generated by OCR assays were analyzed by two-way repeated measures ANOVA followed by Bonferroni's multiple comparison. The specific tests that were employed and additional information can be found in the legend for each Figure.

### Example 8: Primary Neuron Assays for Inner Mitochondrial Potential and ATP Generation

The purpose of this example is to demonstrate the measurement of ΔΨₘ and then ATP production in cultured neurons to identify chemicals that increase the functional output of mitochondria. The inner mitochondrial membrane potential (ΔΨₘ) and proton gradient generated by proton pumps during oxidative phosphorylation (OXPHOS) provide the energy to drive ATP synthesis. ΔΨₘ is widely used to measure the functional status and integrity of mitochondria (N. M. C. Connolly et al., Cell Death Differ. 25 (2018) 542-572; Perry et al., Biotechniques. 50 (2011) 98-115; Zorova et al., Anal Biochem. 552 (2018) 50-59). Hyperpolarization of ΔΨₘ is associated with higher energy demand, as found in differentiating neuroblasts (Voccoli et al., Brain Res. 1252 (2009)15-29), dividing versus quiescent cells (Huang, et al., J. Biomol. Screen. 7 (2002) 383-389), and developmentally active cells (Daniele et al., Sci Rep. 7(1) (2017) 6749; Medina et al., FEBS Lett. 510 (2002) 127-132). Depolarization of ΔΨₘ is associated with diseased cells, aging, apoptosis and toxic insults (Lezi et al., Adv Exp Med Biol. 942 (2012) 269-286; Nadanaciva et al., Curr Protoc Toxicol. Chapter 3, Unit 3.11 (2011); Nicholls, Aging Cell. 3 (2004) 35-40; Wagner et al., Nat Biotechnol. 26 (2008) 343-351).

The fluorescent dye TMRM was used at non-quenching concentrations for measuring ΔΨₘ because of its fast equilibration time, low nonspecific binding to the plasma membrane and reduced toxicity compared to the alternatives of TMRE, Rh123, JC-1 and DiOC₆ (FIG. 16A; FIGS. 13A and 13B) (Connolly et al., 2018; Perry et al., 2011). We performed multiple control experiments before and during our screen to validate, optimize and provide quality control for the TMRM assay including: (i) identifying quenching and non-quenching TMRM concentrations in primary neurons (FIGS. 13A and 13B) and selecting the non-quenching, 10 nM concentration of TMRM for further experiments; (ii) establishing that the necessary wash steps did not alter the integrity of the delicate plated neurons and are required for accurate TMRM fluorescence readings (FIGS. 13C and 13D), measuring TMRM fluorescence from neurons treated with positive, neutral, and negative compounds to show their distinct effects (FIG. 13E), (iii) measuring TMRM fluorescence across 384-well plates treated with DMSO-vehicle alone to identify and adjust for well position effects (FIGS. 13F and 13G), (iv) monitoring DMSO-treated wells during the screen to track variability between replicates and across the eight different source plates used for the screen (FIGS. 13H-13I″), and (v) developing appropriate statistical procedures for data normalization and hit selection (FIGS. 13J-13N). We further optimized a luciferase-based ATP assay as an orthogonal screen to help extract artifacts obtained from the TMRM screen and to monitor the relationship between ΔΨₘ measured with TMRM and ATP concentration in primary cultured neurons (FIGS. 13O-13Q).

For the primary screen, we incubated DIV13 primary neurons with 10 nM TMRM for 90 m to reach equilibrium before pin-tooling compounds from the Spectrum Collection from MicroSource. This library contains 2400 structurally diverse compounds, the majority of which are marketed drugs or compounds in clinical trials. All compounds were assayed in quadruplicate at 12.5 μM, and mitochondria-localized TMRM fluorescence (FIGS. 16A and 16A″) was measured at 4 h and 24 h to identify compounds that have acute or delayed effects on ΔΨₘ (FIG. 16B). We predicted that acute hits might be compounds that directly and rapidly target the electron transport chain to modulate the efficiency of OXPHOS, while compounds with a delayed effect might modulate gene expression or signaling pathways to enhance mitogenesis and/or OXPHOS activities. From the 2400 tested compounds, 16 (0.6%) were selected as putative hits increasing TMRM fluorescence at 4 h and 135 compounds (5.6%) increasing TMRM fluorescence at 24 h. Eleven of the compounds exhibited significant effects at both 4 and 24 h (>3 Z-scores for the inner wells, >2.5 Z-scores for the outer wells, FIGS. 16C-16D″).

We rescreened all 4 and 24 h TMRM hits alongside 40 DMSO control wells and 134 TMRM negative compounds randomly selected from the original screen in a blind fashion. There was a correlation of compound effect size on 24 h TMRM fluorescence between the primary screen and rescreen (R²=0.63, FIG. 16E). 120 of the 135 compounds were confirmed as hits enhancing TMRM fluorescence at 24 hr (89%). Of these, 112 (93%; Table 3) proved positive using the orthogonal assay for ATP production, elevating ATP levels by ˜15% (>3 Z-scores) compared to within-plate DMSO controls (FIG. 16F).

TABLE 3
Listing of the 135 putative hits with Z-scores from the primary screen when assayed
at 24 h (TMRM Primary, hits: >3 Z-scores for inner wells, >2.5 Z- scores
for outer wells), from the rescreen (TMRM Rescreen, hits: >3 Z-scores) and from
the ATP orthogonal screen (ATP, hits with >3 Z-scores). Compounds noted by an
asterisk (*) were hits when assayed at 4 h as well in the 24 h primary screen.
Primary hits that were confirmed in the rescreen (120) are listed above the bold
line. The 112 hits from the ATP orthogonal screen are listed above the dashed line.

| | TMRM | TMRM | |
|---|---|---|---|
| Compound Name | Primary | Rescreen | ATP |
| azelastine hydrochloride | 7.06 | 6.13 | 5.30 |
| pizotyline malate | 3.21 | 5.84 | 7.13 |
| doxepin hydrochloride | 3.44 | 5.41 | 7.03 |
| trimipramine maleate | 5.18 | 5.16 | 5.24 |
| orphenadrine citrate | 3.26 | 5.75 | 6.08 |
| nortriptyline hydrochloride | 3.77 | 5.25 | 5.66 |
| cyclizine | 3.41 | 5.91 | 5.18 |
| ketotifen fumarate* | 3.91 | 3.98 | 5.58 |
| chlorprothixene hydrochloride | 6.17 | 5.04 | 3.17 |
| pimethixene maleate | 3.95 | 5.12 | 4.56 |
| dimenhydrinate | 3.98 | 4.31 | 4.94 |
| cyclobenzaprine hydrochloride | 5.52 | 4.71 | 3.49 |
| clemizole hydrochloride | 3.54 | 5.90 | 4.16 |
| trimeprazine tartrate | 3.13 | 3.94 | 5.75 |
| promazine hydrochloride | 3.81 | 4.04 | 4.76 |
| clozapine | 3.24 | 5.80 | 3.88 |
| thonzylamine hydrochloride | 3.16 | 3.21 | 5.17 |
| chloropyramine hydrochloride | 3.35 | 5.24 | 3.26 |
| trihexyphenidyl hydrochloride | 6.07 | 7.08 | 5.95 |
| procyclidine hydrochloride | 4.55 | 6.91 | 7.14 |
| pridinol methanesulfonate | 5.88 | 6.92 | 5.46 |
| drofenine hydrochloride | 3.68 | 5.96 | 4.84 |
| clidinium bromide | 2.49 | 4.11 | 6.21 |
| piperidolate hydrochloride | 3.58 | 3.76 | 5.72 |
| pipenzolate bromide | 3.14 | 7.09 | 3.45 |
| adiphenine hydrochloride | 3.05 | 3.21 | 4.95 |
| dyclonine hydrochloride | 3.64 | 6.30 | 7.04 |
| dibucaine hydrochloride | 3.83 | 6.82 | 6.22 |
| benoxinate hydrochloride | 3.62 | 6.64 | 5.57 |
| hycanthone | 4.40 | 5.48 | 4.61 |
| tolperisone hydrochloride | 3.57 | 3.67 | 5.49 |
| proparacaine hydrochloride | 3.43 | 4.65 | 4.11 |
| 5alpha-cholestan-3beta-ol-6-one | 7.10 | 7.61 | 5.86 |
| cyclopamine | 3.74 | 8.14 | 5.15 |
| androsterone | 3.77 | 4.76 | 5.63 |
| 5,4′-dimethoxy-7-hydroxyisoflavone | 5.24 | 6.90 | 5.76 |
| phenyl aminosalicylate | 4.63 | 4.94 | 7.48 |
| genistein | 3.38 | 4.21 | 5.96 |
| apigenin dimethyl ether | 3.69 | 5.03 | 4.73 |
| ketanserin tartrate | 5.62 | 5.26 | 5.68 |
| ritanserin | 5.25 | 5.83 | 4.40 |
| domperidone | 3.33 | 5.58 | 5.20 |
| pimozide | 2.65 | 3.74 | 6.60 |
| carvedilol phosphate | 5.81 | 5.43 | 6.87 |
| naftopidil | 3.64 | 9.07 | 3.62 |
| carvedilol | 4.11 | 5.71 | 5.32 |
| acetophenazine maleate | 5.64 | 5.34 | 5.65 |
| piperacetazine | 5.30 | 5.01 | 5.80 |
| thiothixene | 3.08 | 3.38 | 3.80 |
| dihydrofissinolide | 3.94 | 4.61 | 5.15 |
| 1,7-dideacetoxy-1,7-dioxo-3-deacetylkhivorin | 3.65 | 4.00 | 5.46 |
| carapin-8(9)-ene | 3.50 | 3.82 | 4.89 |
| yohimbine hydrochloride | 2.70 | 5.70 | 3.11 |
| rauwolscine hydrochloride | 4.30 | 8.77 | 8.40 |
| reserpine | 7.44 | 7.89 | 5.29 |
| dexpropranolol hydrochloride [R(+)] | 4.37 | 4.79 | 6.22 |
| propranolol hydrochloride (+/−) | 4.03 | 3.07 | 5.06 |
| lidocaine hydrochloride | 3.06 | 4.69 | 6.98 |
| bupivacaine hydrochloride | 5.35 | 5.25 | 4.80 |
| toremifene citrate | 3.31 | 7.05 | 3.29 |
| clomiphene citrate | 2.94 | 3.45 | 4.20 |
| estradiol methyl ether | 3.74 | 3.55 | 4.80 |
| estrone acetate | 3.26 | 5.89 | 3.36 |
| celecoxib* | 6.67 | 4.72 | 7.57 |
| diperodon hydrochloride | 3.75 | 6.07 | 8.02 |
| vinpocetine | 5.76 | 6.16 | 6.29 |
| nefazodone hydrochloride | 4.00 | 6.11 | 6.30 |
| butacaine sulfate | 3.85 | 5.06 | 6.99 |
| nebivolol hydrochloride | 3.73 | 5.29 | 6.92 |
| lobeline hydrochloride | 6.62 | 6.75 | 3.67 |
| indole-3-carbinol | 3.15 | 6.27 | 6.52 |
| trimebutine maleate* | 3.79 | 5.82 | 6.14 |
| tepoxalin | 4.36 | 3.75 | 6.89 |
| meprylcaine hydrochloride | 3.56 | 5.91 | 6.05 |
| nimodipine | 4.40 | 3.98 | 6.53 |
| penfluridol | 4.29 | 4.83 | 5.75 |
| bisphenol a* | 3.35 | 7.40 | 4.73 |
| nafronyl oxalate* | 4.08 | 5.89 | 5.17 |
| doxazosin mesylate | 4.39 | 5.94 | 4.68 |
| benzonatate | 2.78 | 8.21 | 4.35 |
| tigecycline | 3.91 | 5.95 | 4.97 |
| ajmaline | 3.76 | 6.40 | 4.76 |
| pyrimethamine* | 3.43 | 4.00 | 6.54 |
| exalamide | 3.39 | 4.57 | 6.10 |
| hydroxyzine pamoate | 3.15 | 6.04 | 5.22 |
| mefloquine | 3.75 | 5.54 | 5.09 |
| tiletamine hydrochloride | 3.31 | 4.20 | 6.27 |
| ambroxol hydrochloride | 4.00 | 5.14 | 5.04 |
| colistin sulfate | 3.27 | 5.24 | 5.48 |
| heteropeucenin, methyl ether | 3.45 | 4.33 | 5.92 |
| propafenone hydrochloride | 3.75 | 5.04 | 5.18 |
| quinine ethyl carbonate | 4.64 | 6.26 | 3.60 |
| aripiprazole | 2.98 | 7.76 | 3.66 |
| fulvestrant | 3.31 | 6.27 | 4.27 |
| bussein | 3.44 | 3.92 | 5.58 |
| canagliflozin | 4.55 | 6.36 | 3.13 |
| alverine citrate | 4.24 | 4.15 | 4.56 |
| doxorubicin | 4.02 | 5.05 | 4.00 |
| sclareol | 2.80 | 5.41 | 4.64 |
| imidazol-4-ylacetic acid sodium salt* | 3.58 | 4.56 | 4.61 |
| oxiconazole nitrate | 3.08 | 3.28 | 5.76 |
| naftifine hydrochloride | 3.26 | 4.64 | 4.65 |
| sertraline hydrochloride | 3.42 | 4.15 | 4.82 |
| 1-hydroxy-3,6,7-trimethoxy-2,8-diprenylxanthone | 3.34 | 3.27 | 5.44 |
| dehydroabietamide | 3.42 | 5.12 | 4.15 |
| eugenol* | 4.17 | 3.10 | 4.39 |
| penbutolol sulfate | 3.16 | 5.35 | 3.39 |
| paroxetine hydrochloride | 3.53 | 3.26 | 4.41 |
| butyl paraben | 3.42 | 4.18 | 3.84 |
| triclabendazole | 3.30 | 3.61 | 3.97 |
| clemastine fumarate | 2.82 | 3.22 | 4.56 |
| levomilnacipran hydrochloride | 3.26 | 3.53 | 3.87 |
| sodium nitroprusside* | 3.38 | 3.22 | 2.90 |
| medroxyprogesterone acetate* | 3.66 | 3.67 | 2.64 |
| estradiol cypionate | 2.59 | 4.67 | 2.98 |
| avanafil | 3.13 | 3.42 | 1.30 |
| terconazole | 5.15 | 5.95 | 2.82 |
| oxelaidin citrate | 4.27 | 6.44 | 2.73 |
| estriol benzyl ether | 3.17 | 6.67 | 2.03 |
| larixol acetate | 3.96 | 5.67 | 2.67 |
| amitriptyline hydrochloride | 4.78 | 1.81 | 4.82 |
| imipramine hydrochloride | 3.31 | 0.91 | 5.10 |
| benazepril hydrochloride | 3.43 | 2.43 | 5.26 |
| fluoxetine hydrochloride | 3.42 | 1.87 | 3.70 |
| orlistat | 3.43 | 1.56 | 2.36 |
| bifonazole | 3.81 | 1.84 | 4.00 |
| felodipine | 3.80 | 2.41 | 5.08 |
| ancitabine hydrochloride | 3.04 | 1.36 | 0.14 |
| phytol | 3.42 | 2.70 | 4.63 |
| 4′-methoxychalcone | 3.42 | 1.48 | 3.97 |
| desoxycorticosterone acetate* | 2.81 | 1.08 | 3.62 |
| sparteine sulfate | 2.65 | 1.95 | 2.92 |
| chlorpromazine | 2.79 | 2.66 | 5.00 |
| nicardipine hydrochloride | 4.22 | −5.52 | −3.75 |
| pramoxine hydrochloride | 2.93 | −4.72 | −1.97 |

Although the correlation of ATP and TMRM effect sizes was more modest (R²=0.46), the putative hits significantly enhanced TMRM fluorescence and ATP production compared to non-hit compounds or DMSO-treated controls (FIG. 16F). Only 2 of the 16 (13%) compounds that elevated TMRM fluorescence at 4 h increased ATP levels. To probe the relationship between TMRM signal and ATP production, we compared the correlation coefficient between the TMRM fluorescence of all rescreened compounds and ATP production at 4 and 24 h. We found a very weak correlation between these two parameters for 4 h of incubation (R²=0.16, P<0.0001, FIG. 13Q) and a modest correlation (R²=0.46, P<0.0001, FIG. 16F) for 24 h, indicating a general trend between the measured whole-well TMRM signal and higher energetics of the neuronal population. We focused our subsequent studies on hits selected after a 24 hr incubation with neurons given the stronger relationship between TMRM fluorescence and ATP generation. Using this incubation time, the results indicate that increased A ΔΨₘ is a good, inexpensive surrogate for detecting elevated levels of ATP within neurons.

### Example 9: Structural and Functional Diversity and Potency of Modulators of Mitochondrial Function

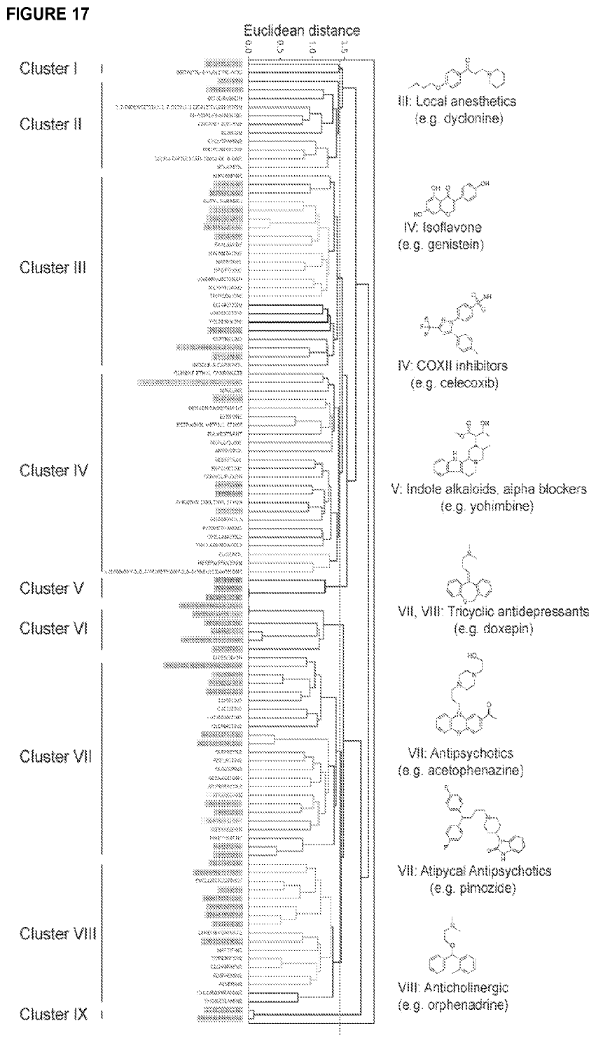

Many of the confirmed 24 h TMRM/ATP hits were structurally related and so they were grouped using hierarchical clustering and Tanimoto similarities into several clusters (FIG. 17). We also discovered that some of the structural clusters contain compounds with a similar therapeutic use (Table 4). Surprisingly, 9 of the hit compounds are topical/local anesthetics (primarily in Cluster 3, orange shading in FIG. 17) that increase neuronal mitochondrial ΔΨₘ and elevate ATP. Due to this novel finding, we included representatives from this group in subsequent experiments (lidocaine, benoxinate, dyclonine). Although only two isoflavone compounds were identified (Cluster 4, green), genistein was reported (Ding et al., Basic Clin Pharmacol Toxicol. 108 (2011) 333-340) to increase ΔΨₘ in primary cortical neurons treated with Aβ peptide. Therefore, we employed genistein in latter experiments as a putative positive control compound. Cluster 4 also contains many steroid hormone receptor modulators and two COX inhibitors (grey). Estrogen derivatives were previously shown to contain neuro- and mitochondria-protective activity (Mortibouys et al, Brain. 136 (2013) 3038-3050; Sherman et al., Dis Model Mech. 11(2) dmm031906 (2018); Grimm et al., 2014, Irwin et al., 2008; Nilsen et at, 2007), but the mitochondrial effect of COX inhibitors was unexpected. Thus, we chose celecoxib for further characterization. Cluster 5 is comprised of three indole alkaloids which have alpha-blocker activity (brown). One of them, yohimbine was previously found to be neuroprotective by inducing the NRF2-mediated antioxidant response (WO2012149478A3). Therefore, we included yohimbine in follow-up experiments as a potential positive control and to explore this activity further. Cluster 6 contains a collection of structurally similar alpha/beta blockers for the treatment of high blood pressure and heart diseases (anti-adrenergics, brown), from which we chose carvedilol and naftopidil for further tests. We also highlight the interesting identification of several antipsychotics and classic tricyclic antidepressants (pink), anti-cholinergics (purple) and anti-dopaminergic/anti-serotoninergic compounds (yellow) in clusters 7 and 8, whose unanticipated functional modulation of mitochondria made them intriguing compounds for additional experiments (Table 4).

TABLE 4
Represents the maximal percent increase of TMRM fluorescence or ATP production relative to in-plate DMSO
controls from dose response experiments. Data were generated from 6 independent experiments, with each
compound tested in duplicate for each experiment. Significant changes in orthogonal assays are indicated,
unless they were non-significant (n.s.) or not measured (—). OCR: oxygen consumption rate.

| | | TMRM- | ATP- | TMRM | ATP | | | | | 3xTG |
|---|---|---|---|---|---|---|---|---|---|---|
| | | EC₅₀ | EC₅₀ | (Effect- | (Effect- | | Mitochondrial | Neurite | Luperox | genetic |
| Cluster | Compound | (nM) | (nM) | size) | size) | OCR | Morphology | Area | insult | background |
| Oral/Local | Dyclonine | 750 | 140 | 22.0% | 20.7% | increased | elongated | increased | rescues | rescues |
| Anesthetic | Benoxinate | 490 | 80 | 21.0% | 21.4% | — | elongated | n.s. | rescues | rescues |
| (cluster 3) | Lidocaine | 1890 | 110 | 17.4% | 15.8% | — | — | — | rescues | rescues |
| Antibiotic | Phenyl-4-amino | 1800 | 2020 | 21.8% | 20.4% | — | elongated | n.s. | rescues | rescues |
| (cluster 3) | salicylate | | | | | | | | | |
| Isoflavone | Genistein | 1300 | 3000 | 19.5% | 20.6% | — | elongated | n.s. | rescues | rescues |
| (cluster 4) | | | | | | | | | | |
| COX | Celecoxib | 5180 | 490 | 27.5% | 20.7% | — | elongated | n.s. | no | rescues |
| Inhibitor | | | | | | | | | rescue | |
| (cluster 4) | | | | | | | | | | |
| Indole | Yohimbine | 620 | 270 | 18.6% | 23.6% | increased | elongated | increased | rescues | rescues |
| Alkaloids | Rauwolscine | 4580 | 1000 | 29.4% | 20.9% | — | — | — | — | — |
| (cluster 5) | Reserpine | 640 | 120 | 22.5% | 20.5% | — | — | — | — | — |
| Adrenergic | Carvedilol | 350 | 230 | 20.2% | 15.9% | — | — | — | — | — |
| Antagonists | Naftopidil | 2240 | 230 | 18.8% | 15.0% | — | — | — | — | — |
| (cluster 6) | | | | | | | | | | |
| Anti-psychotics | Penfluridol | 50 | 30 | 25.2% | 22.4% | — | elongated | increased | no | rescues |
| Dopamine | | | | | | | | | rescue | |
| Serotoninergic | Pimozide | 130 | 90 | 25.1% | 20.4% | — | elongated | increased | rescues | rescues |
| Antagonists | Ketanserin | 1300 | 160 | 30.0% | 19.1% | — | — | — | — | — |
| (cluster 7) | Domperidone | 1800 | 65 | 28.3% | 13.0% | — | — | — | — | — |
| Tricyclic | Pizotyline | 2600 | 360 | 25.2% | 17.9% | — | — | — | — | — |
| Antidepressant | Trimipramine | 360 | 180 | 20.0% | 16.4% | — | — | — | — | — |
| (clusters 7 | Doxepin | 2100 | 170 | 26.1% | 18.3% | increased | elongated | n.s. | rescues | rescues |
| and 8) | Nortriptyline | 550 | 200 | 21.0% | 16.5% | — | — | — | — | — |
| Typical | Acetophenazine | 1150 | 300 | 17.5% | 16.7% | — | — | — | — | — |
| Anti-psychotic | | | | | | | | | | |
| phenothiazine | | | | | | | | | | |
| (cluster 7) | | | | | | | | | | |
| Cholinergic | Orphenadrine | 2250 | 180 | 25.5% | 14.3% | — | — | — | — | — |
| Antagonist | Pridinol | 3900 | 1650 | 20.6% | 15.7% | — | — | — | — | — |
| (cluster 7, | Clidinium | 450 | 420 | 14.3% | 20.5% | — | — | — | — | — |
| 8, 9) | Procyclidine | 850 | 240 | 23.3% | 18.7% | — | — | — | — | — |
| | Trihexyphenidyl | 3200 | 170 | 20.0% | 17.8% | — | — | — | — | — |

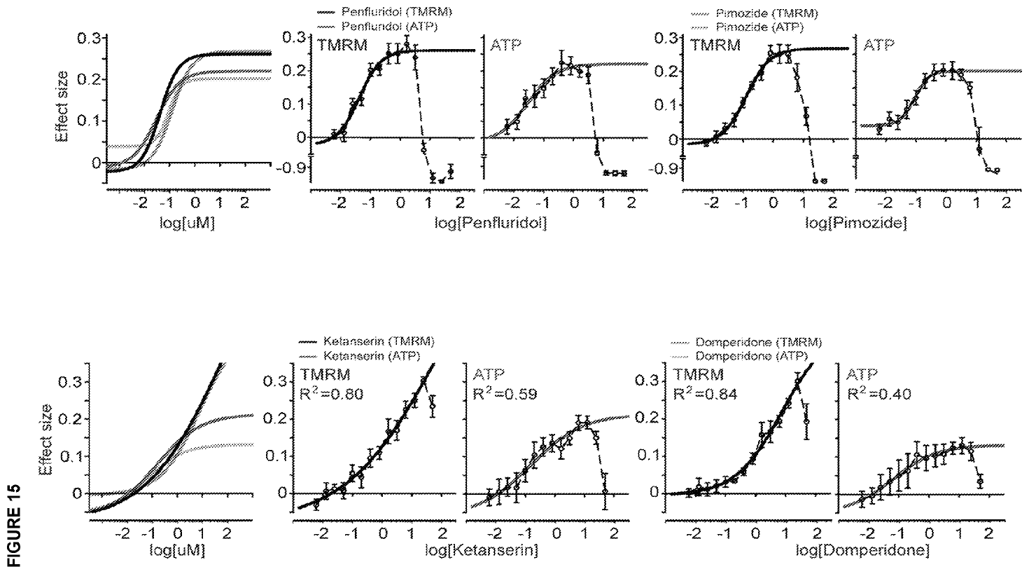

Dose response assays (FIGS. 14 and 15; Table 4) of selected compounds revealed that the EC₅₀'s for TMRM fluorescence and/or ATP enhancement were generally between 0.1-5 μM. Effect sizes were measured between 15-30%. Nearly all compounds, apart from yohimbine, exhibited inverted-U shaped responses suggesting mitochondrial toxicity at high concentrations. Neurons are highly sensitive cells and most compounds have detrimental effects at high concentrations.

### Example 10: Mitochondrial Functional Modulators Potentiate Respiration

The purpose of this example is to demonstrate that the compounds identified by the present disclosure increase mitochondrial respiration. We selected yohimbine (indole alkaloid) and doxepin (tricyclic antidepressant) for the necessary and more complex, absolute oxygen consumption rate (OCR) experiments. OCR at baseline and in response to excess ADP/substrate (State3/3u) were measured and normalized to protein content (Salabei et al., 2014; Sims and Anderson, 2008) using primary neurons presented with compounds for 24 h.

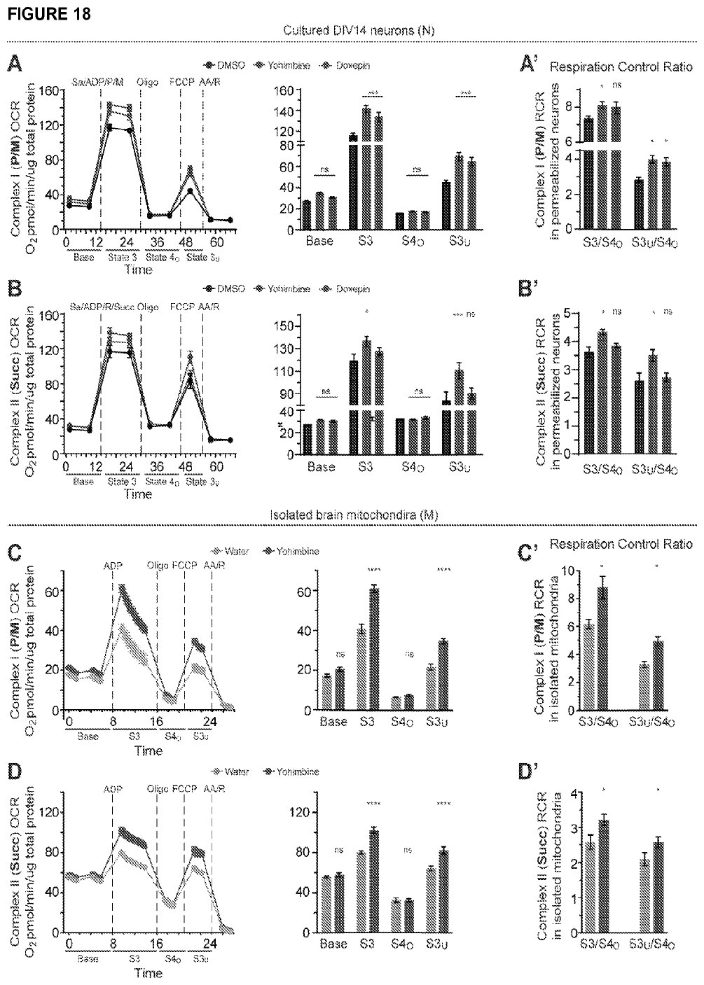

Primary neurons exposed to yohimbine or doxepin showed a weak, non-significant, but consistent trend towards an increased basal rate of respiration, potentially due to a modest increase in mitochondrial function and/or content (FIGS. 18A-18B′). More importantly, we observed a significant increase of State 3 and 3u respiration stimulated by either Complex I or Complex II substrates in mitochondria from cultured neurons treated with yohimbine (FIGS. 18A-18B′), and a significant increase with Complex I substrates for doxepin.

We also took advantage of the water solubility of yohimbine to administer this compound to mice in drinking water across a period of 7 months. A similar respiratory increase was observed in mitochondria isolated from the whole brains of animals chronically treated with yohimbine (FIGS. 18C and 18D). We calculated the Respiratory Control Ratios (RCR, State 3/4ₒ) to provide a broad index of mitochondrial function (Brand and Nicholls, 2011; Rogers et al., 2011). State 3 respiration measures the maximal ability of mitochondria for substrate oxidation and ATP generation. State with OCR measured in response to oligomycin challenge, measures proton leak. The ratio between these states is influenced by most functions of OXPHOS, thus providing a net measure of the tightness between respiration and phosphorylation. Yohimbine treatment significantly increased RCR with both Complex I and II substrates with both mitochondria from cultured neurons and isolated brain mitochondria. (FIGS. 18A′, 18B′, 18C′, and 18D′). Doxepin weakly increased RCR using Complex I but not II substrates in cultured neurons (FIGS. 18A′-18B′). Collectively, the enhanced State 3 respiration and RCR with yohimbine and the weaker effects of doxepin indicate more efficient substrate oxidation, electron transport, coupling to OXPHOS and a lower proton leak, indicating a higher respiration capacity and efficiency in the treated mitochondria. Moreover, we previously reported that the anesthetic dyclonine produces beneficial effects in similar cell culture and in vivo experiments (Varkuti et al., 2020).

Thus, representative compounds from three classes of functional mitochondrial modulators—indole alkaloids, tricyclic antidepressants, and local/topical anesthetics—all enhance neuronal mitochondrial function. These modulators may act directly on mitochondria or indirectly by stimulating cell signaling pathways that influence mitochondrial function (Sherman and Bang, 2018; Tsvetkov et al., 2010). However, the functional changes on mitochondria instilled by the tested compounds must be enduring, and become independent of cytoplasmic signaling pathways, since increased function persists in permeabilized neurons and in mitochondria purified from cells.

### Example 11: Modulators of Neuronal Mitochondrial Function Alter Mitochondrial Morphology

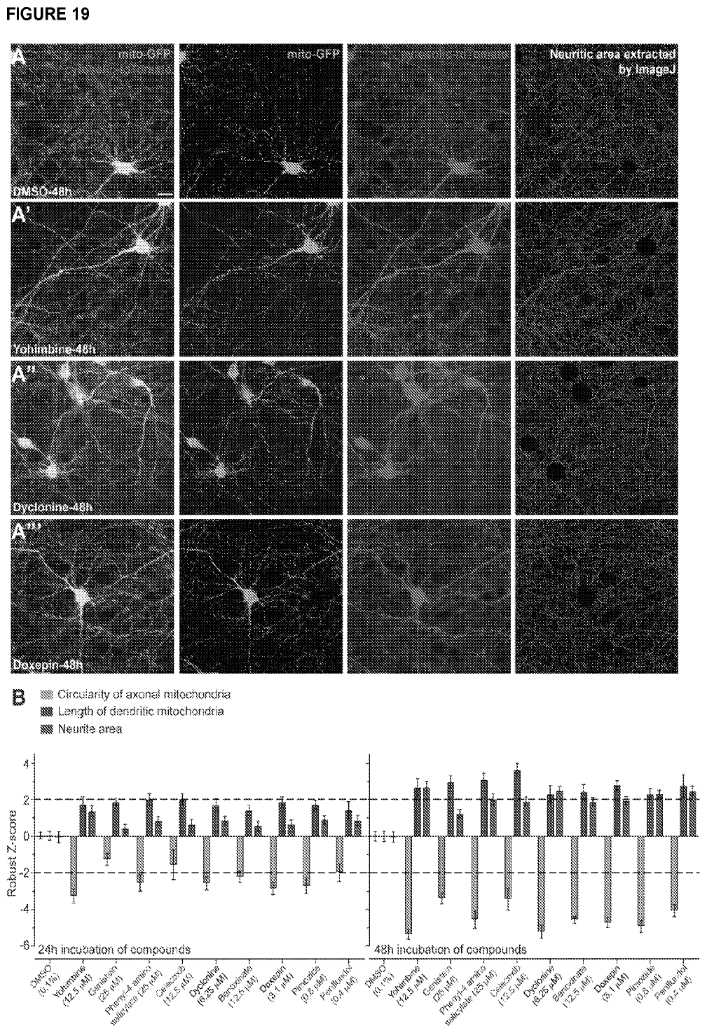

The purpose of this example is to demonstrate that enhanced mitochondrial function is associated with mitochondrial morphological changes. A high-content assay was developed in parallel to monitor aspects of mitochondrial dynamics in primary cultured neurons that conditionally express (Cre recombinase dependent) mitochondrial-tagged GFP and cytosolic tdTomato reporters (Varkuti et al., 2020). We monitored two aspects of neuritic mitochondrial dynamics: (1) the health of mitochondria as measured by the average circularity of axonal mitochondria, since defective mitochondria targeted for mitophagy become circular in shape, and (2) the balance between fission/fusion measured by the average length of dendritic mitochondria. We also determined whether the compounds induced neurite sprouting by measuring the total area of neurites within the collected images. FIG. 19A illustrates the results of surveying a collection of functional modulators selected from 5 different structural clusters (FIG. 17), quantified as robust z-scores relative to DMSO-treated control neurons.

All tested compounds showed a trend towards promoting more oblong mitochondria after 24 h and they exhibited significant potency (Z-score <−2.0) compared to the control after 48 h of treatment. Notably, the identified mitochondrial OCR enhancers yohimbine, dyclonine and doxepin exhibited robust effects. These results provide evidence that the functional modulators promote healthier mitochondria using the morphological surrogate of circularity.

The tested compounds also promoted the lengthening of dendritic mitochondria to Z-scores 2.0 after 48 h of treatment. This observation is consistent with a report that mitochondrial fusion and fragmentation are associated with higher and lower ΔΨₘ values, respectively, in neuroblasts and stable cell lines (Sherman et al., 2018; Voccoli and Colombaioni, 2009). Yohimbine, dyclonine, pimozide and penfluridol were also observed to increase neuritic area after 48 h of treatment.

### Example 12: Neuronal Mitochondrial Modulators Provide Protection Against Insults Associated with Neurodegenerative Disorders

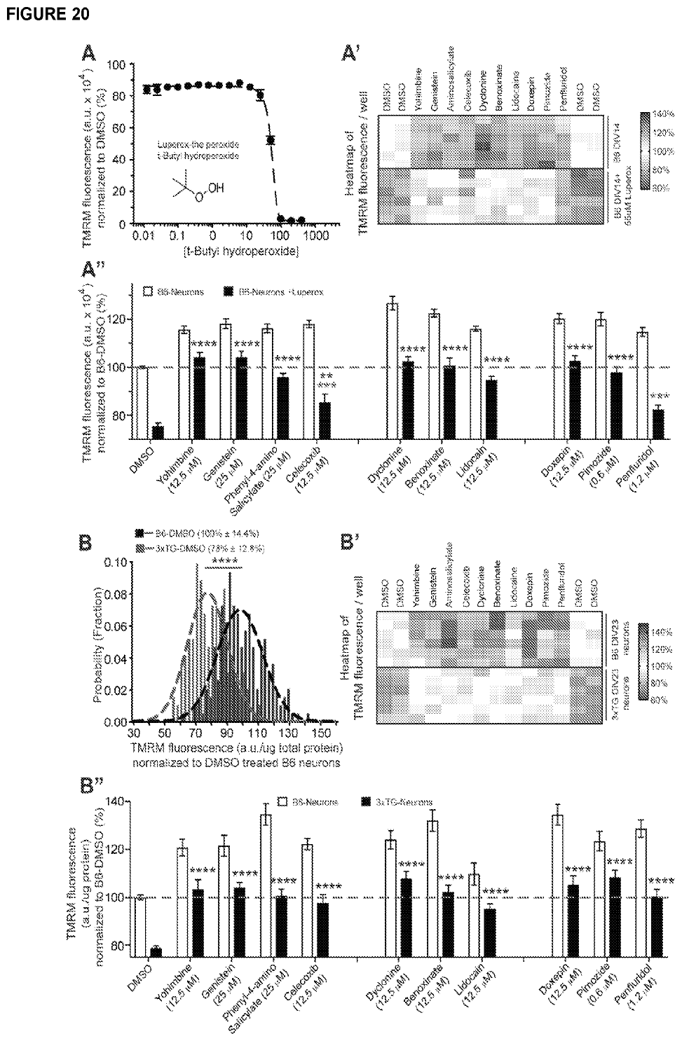

Given the strong relationship between mitochondrial dysfunction and brain disorders, we tested a group of functional mitochondrial modulators for their potential to protect against two neurodegeneration-associated insults: neurons subjected to increased oxidative stress and those expressing Alzheimer's-causing gene variants (neurons from 3×TG mice). Adding tert-butyl hydroperoxide to the cultures to increase oxidative stress severely reduced TMRM fluorescence at concentrations above 50 μM and led to complete cell death at concentrations >100 μM (FIG. 20). Eight of the 10 tested compounds provided protection to the ΔΨₘ from the toxic effects of increased oxidative stress (FIGS. 20A-20A″). Primary neurons isolated and cultured from 3×TG mice to DIV23 exhibited a ˜20% reduction in TMRM fluorescence normalized to protein content per well when compared to C57Bl/6J neurons (FIGS. 20B-20B″). All compounds tested significantly increased ΔΨₘ in the presence of the insult to the level observed for control B6 neurons (FIG. 20B″). Remarkably, many of the tested compounds protect against both insults, including yohimbine, genistein, dyclonine, benoxinate, lidocaine, phenyl-4-amino salicylate, pimozide and doxepin (FIGS. 20A″ and 20B″).

### Example 13: In Vivo Efficacy of Identified Drugs on Neuronal Mitochondria in Alzheimer's Disease Model

The purpose of this example is to demonstrate that drugs (MnMs) identified in the screens described herein (e.g., Examples 1-3) exhibited in vivo efficacy in protecting the mitochondrial population in neurons in models of Alzheimer's Disease (AD).

To assay representative compounds in this example, we employed a Drosophila model for AD (X. Wang et al., Mol. Neurobiol. (2020) https://doi.org/10.1007/s12035-020-02107-w). The model employs the expression of the toxic peptide—β42, which is a dominant toxic protein responsible for genetic forms of Alzheimer's disease—in a subset of brain neurons that mediate learning and memory ability (mushroom body neurons). Flies expressing this toxic protein show: (1) small (3-dimensional volume, 3-dimensional surface area, length), rounded (sphericity), and more numerous neuronal mitochondria, (2) dysfunction of mitochondria (calcium import), (3) amyloid plaque formation, (4) increased cell death (apoptosis), and a learning impairment compared to control flies (Wang et al., 2020). Drugs that show efficacy therefore: enlarge neuronal mitochondria (increased volume, increased surface area, increased length), reduce sphericity, and reduce the number of mitochondria compared to untreated Alzheimer's flies.

The AD model flies were collected immediately after eclosion from the pupal case and transferred to food that was laced with drug at 100 micromolar concentration. The flies were aged to 15 days-of-age: this is when the mitochondrial pathology is very robust.

We compared the morphology of mitochondria collected from these flies treated with drug and compared them to the mitochondria of flies that remained untreated. In some cases, the mitochondria were compared in the cell bodies of the neurons; in other cases we compared the mitochondria in the axons of neurons (Tables 5 and 6).

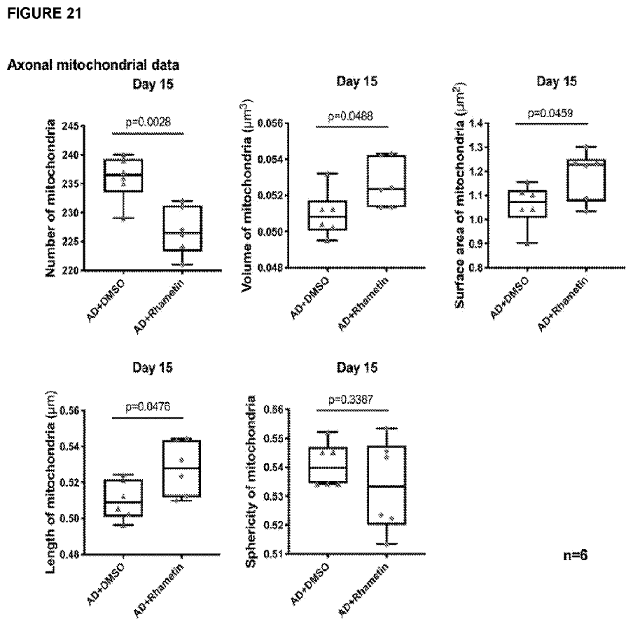

Representative drugs, as identified in the screens, were rhamnetin (FIG. 21), orphenadrine, penfluridol, carvedilol, pimozide, genistein, phenyl-4 amino salicylate, 4-hydroxychalcone, yohimbine, alverine, and naftopidil. The mitochondrial morphological data show that the drugs exhibit some or all of the desired protective properties in cell bodies of neurons (Table 5) and in axons of neurons (Table 6). Each table shows the probability that the parameter compared between untreated and treated Alzheimer's flies differs solely on the basis of chance. A probability of p<0.05 indicates that there exists a significant difference between the mitochondria of treated and untreated AD flies.

TABLE 5
Mitochondria measured in the cell bodies of neurons

| Drug Name | Number | Volume | Surface area | Sphericity | Length | n |
|---|---|---|---|---|---|---|
| Rhamnetin | p = 0.0117 | p = 0.0631 | p = 0.0564 | p = 0.3023 | p = 0.0641 | 6 |
| orhphenadrine | p = 0.0037 | p = 0.04 | p = 0.022 | p = 0.092 | p = 0.02 | 6 |
| Penfluridol | p = 0.0125 | p = 0.08 | p = 0.066 | p = 0.62 | p = 0.07 | 6 |
| Carvedilol | p = 0.05 | p = 0.09 | p = 0.078 | p = 0.51 | p = 0.08 | 6 |
| Pimozide | p = 0.016 | p = 0.0135 | p = 0.017 | p = 0.162 | p = 0.0 | 6 |
| Genistein | p = 0.02; | p = 0.0989 | p = 0.083 | p = 0.72 | p = 0.02; | 6 |
| Phenyl-4 amino | p = 0.0429 | p = 0.5489 | p = 0.6212 | p = 0.7421 | p = 0.6096 | 6 |
| salicylate | | | | | | |

TABLE 6
Mitochondria measured in the axons of neurons.

| Drug Name | Number | Volume | Surface Area | Length | Sphericity | n |
|---|---|---|---|---|---|---|
| Orhphenadrine (1) | p = 0.000034 | p = 0.092 | p = 0.089 | not analyzed | p = 0.123 | 4 |
| 4-OH chalcone | p = 0.000043 | p = 0.00012 | p = 0.00056 | not analyzed | p = 0.223 | 4 |
| Yohimbine | p = 0.00002; | p = 0.098 | p = 0.089 | not analyzed | p = 0.452 | 4 |
| Alverine | p = 0.0132 | p = 0.0144 | p = 0.0154 | not analyzed | p = 0.27 | 4 |
| Naftopidil | p = 0.0134 | p = 0.0152 | p = 0.00023 | p = 0.099 | p = 0.0081 | 4 |
| orhphenadrine (2) | p = 0.0488 | p = 0.0062 | p = 0.0081 | p = 0.0493 | p = 0.14 | 6 |
| Penfluridol | p = 0.0251 | p = 0.0728 | p = 0.067 | p = 0.0268 | p = 0.132 | 6 |
| Carvedilol | p = 0.0472 | p = 0.0220 | p = 0.031 | p = 0.0566 | p = 0.331 | 6 |
| Pimozide | p = 0.0161 | p = 0.0160 | p = 0.022 | p = 0.0258 | p = 0.723 | 6 |
| Genistein | p = 0.0286 | p = 0.0452 | p = 0.0344 | p = 0.0954 | p = 0.347 | 6 |
| Rhamnetin | p = 0.0028 | p = 0.0488 | p = 0.0459 | p = 0.0476 | p = 0.3387 | 6 |

Numbered citations in the present disclosure are as follows:
1. T. Misgeld, T. L. Schwarz, Mitostasis in neurons: maintaining mitochondria in an extended cellular architecture. Neuron. 96, 651-666 (2017).
2. R. M. Whitaker, D. Corum, C. C. Beeson, R. G. Schnellmann, Mitochondrial biogenesis as a pharmacological target: A new approach to acute and chronic diseases. Annu Rev Pharmacol Toxicol. 56, 29-49 (2016).
3. J. Nunnari, A. Suomalainen, Mitochondria: in sickness and in health. Cell. 148, 145-159 (2012).
4. D. Wang, J. Wang, G. M. Bonamy, S. Meeusen, R. G. Brusch, C. Turk, P. Yang, P. g. Schultz, A small molecule promotes mitochondrial fusion in mammalian cells. Angew Chem Int Ed Engl. 51, 9302-9305 (2012).
5. S. H. Baek, S. J. Park, J. I. Jeong, S. H. Kim, J Han, J. W. Kyung, S. H. Baik, Y. Choi Y, B. Y. Choi, J. S. Park, G. Bahn, J. H. Shin, D. S. Jo, J. Y. Lee, C. G. Jang, T. V. Arumugam, J. Kim, J W. Han, J. Y. Koh, D. H. Cho, D. G. Jo, Inhibition of Drp1 ameliorates synaptic depression, abeta deposition, and cognitive impairment in an Alzheimer's disease model. J Neurosci. 37, 5099-5110 (2017).
6. Q. Cai, P Tammineni, Mitochondrial aspects of synaptic dysfunction in Alzheimer's disease. J Alzheimers Dis. 57, 087-1103 (2017).
7. M. J. Calkins, M. Manczak, P. Mao, U. Shirendeb, P. H. Reddy, Impaired mitochondrial biogenesis, defective axonal transport of mitochondria, abnormal mitochondrial dynamics and synaptic degeneration in a mouse model of Alzheimer's disease. Hum Mol Genet. 20, 4515-4529 (2011).
8. C. Desler, M. S. Lillenes, T. Tonjum, L. J. Rasmussen, The role of mitochondrial dysfunction in the progression of Alzheimer's disease. Curr Med Chem. 25, 5578-5587 (2017).
9. D. I. Kim, K H Lee, J. Y. Oh, J. S. Kim, H J Han, Relationship between beta-amyloid and mitochondrial dynamics Cell Mol Neurobiol. 37, 955-968 (2017).
10. J. Lewerenz, P. Maher, Chronic glutamate toxicity in neurodegenerative diseases-what is the evidence? Front Neurosci. 9, 469 (2015).
11. G. H. Kim, J E Kim, S J Rhie, S. Yoon, The role of oxidative stress in neurodegenerative diseases. Exp Neurobiol. 24, 325-340 (2015).
12. L Pagani, A. Eckert, Amyloid-Beta interaction with mitochondria. Int J Alzheimers Dis, 2011, 925050.
13. E. F. Iannetti, J. A. Smeitink, J. Beyrath, P. H. Willems, W. J. Koopman, Multiplexed high-content analysis of mitochondrial morphofunction using live-cell microscopy. Nat Protoc. 11, 1693-1710 (2016).
14. E. F. Iannetti, P. H. Willems, M. Pellegrini, J. Beyrath, J. A. Smeitink, L. Blanchet, W. J. Koopman, Toward high-content screening of mitochondrial morphology and membrane potential in living cells. Int J Biochem Cell Biol. 63, 66-70 (2015).
15. T. Kitami, D. J. Logan, J. Negri, T. Hasaka, N. J. Tolliday, A. E. Carpenter, B. M. Spiegelman, V. K. Mootha, A chemical screen probing the relationship between mitochondrial content and cell size. PLoS One. 7, e33755 (2012).
16. D. F. Suen, K. L. Norris, R. J. Youle, Mitochondrial dynamics and apoptosis. Genes Dev. 22, 1577-1590 (2008).
17. D. Ebrahimi-Fakhari, A. Saffari, L. Wahlster, A. Di Nardo, D. Turner, T. L. Jr. Lewis, C. Conrad, J. M. Rothberg, J. O. Lipton, S. Kölker, G. F. Hoffmann, M. J. Han, F. Polleux, M. Sahin, Impaired mitochondrial dynamics and mitophagy in neuronal models of tuberous sclerosis complex. Cell Rep. 17, 1053-1070 (2016).
18. C. H. Hung, S. S. Cheng, Y. T. Cheung, S. Wuwongse, N. Q. Zhang, Y. S. Ho, S. M. Lee, R. C. Chang, A reciprocal relationship between reactive oxygen species and mitochondrial dynamics in neurodegeneration. Redox Biol. 14, 7-19 (2018).
19. K. K. Badal, K. Akhmedov, P. Lamoureux, X. A. Liu, A. Reich, M. Fallahi-Sichani, S. Swarnkar, K. E. Miller, S. V. Puthanveettil, Synapse formation activates a transcriptional program for persistent enhancement in the bi-directional transport of mitochondria. Cell Rep. 26, 507-517 e3 (2019).
20. R. L. Frederick, J. M. Shaw, Moving mitochondria: establishing distribution of an essential organelle. Traffic. 8, 1668-1675 (2007).
21. R. Tsutsumi, T. Yoshida, Y. Nii, N. Okahisa, S. Iwata, M. Tsukayama, R. Hashimoto, Y. Taniguchi, H. Sakaue, T. Hosaka, E. Shuto, T. Sakai, Sudachitin, a polymethoxylated flavone, improves glucose and lipid metabolism by increasing mitochondrial biogenesis in skeletal muscle. Nutr Metab (Load). 11, 32 (2014).
22. M. Lindahl, C. Tagesson, Flavonoids as phospholipase A2 inhibitors: importance of their structure for selective inhibition of group II phospholipase A2. Inflammation. 21, 347-356 (1997).
23. M. Novo Belchor, H. Hessel Gaeta, C. Fabri Bittencourt Rodrigues, C. Ramos da Cruz Costa, D. de Oliveira Toyama, L. F. Domingues Passero, M. Dalastra Laurenti, M. Hikari Toyama, Evaluation of Rhamnetin as an inhibitor of the pharmacological effect of secretory phospholipase A2. Molecules. 22, 9 (2017).
24. A. A. Farooqui, W. Y. Ong, L. A. Horrocks, Inhibitors of brain phospholipase A2 activity: their neuropharmacological effects and therapeutic importance for the treatment of neurologic disorders. Pharmacol Rev. 58, 591-620 (2006).
25. J. W. Choi, B. K. Jang, N. C. Cho, J. H. Park, S. K. Yeon, E. J. Ju, Y. S. Lee, G. Han, A. N. Pae, D. J. Kim, K. D. Park, Synthesis of a series of unsaturated ketone derivatives as selective and reversible monoamine oxidase inhibitors. Bioorg Med Chem. 23, 6486-6496 (2015).
26. D. E. Edmondson, Hydrogen peroxide produced by mitochondrial monoamine oxidase catalysis: biological implications. Curr Pharm Des. 20, 155-160 (2014).
27. J. Zheng, V. D. Ramirez, Inhibition of mitochondrial proton F0F1-ATPase/ATP synthase by polyphenolic phytochemicals. Br J Pharmacol. 130, 1115-1123 (2000).
28. T. P. Nguyen, P. Nioi, C. B. Pickett, The Nrf2-antioxidant response element signaling pathway and its activation by oxidative stress. J Biol Chem. 284, 13291-13295 (2009).
29. K. M. Holmstrom, R. V. Kostov, A. T. Dinkova-Kostova, The multifaceted role of Nrf2 in mitochondrial function. Curr Opin Toxicol. 1, 80-91 (2016).
30. C. M. Klinge, Estrogenic control of mitochondrial function and biogenesis. J Cell Biochem. 105, 1342-1351 (2008).
31. F. R. Jornayvaz, G. I. Shulman, Regulation of mitochondrial biogenesis. Essays Biochem. 47, 69-84 (2010).
32. L. A. Arnold, E. Estébanez-Perpiñá, M. Togashi, A. Shelat, C. A. Ocasio, A. C. McReynolds, P. Nguyen, J. D. Baxter, R. J. Fletterick, P. Webb, R. K. Guy, A high-throughput screening method to identify small molecule inhibitors of thyroid hormone receptor coactivator binding. Sci STKE. 2006. 341, stke.3412006.
33. V. Choudhary, I. Kaddour-Djebbar, V. Lakshmikanthan, T. Ghazaly, G. S. Thangjam, A. Sreekumar, R. W. Lewis, I. G. Mills, W. B. Bollag, M. V. Kumar, Novel role of androgens in mitochondrial fission and apoptosis. Mol Cancer Res. 9, 1067-1077 (2011).
34. B. K. Singh, R. A. Sinha, M. Tripathi, A. Mendoza, K. Ohba K, J. A. C. Sy, S. Y. Xie, J. Zhou, J. P. Ho, C. Y. Chang, Y. Wu, V. Giguere, B. H. Bay, J. M. Vanacker, S. Ghosh, K. Gauthier, A. N. Hollenberg, D. P. McDonnell, P. M. Yen, Thyroid hormone receptor and ERRalpha coordinately regulate mitochondrial fission, mitophagy, biogenesis, and function. Sci Signal. 11, 536 (2018).
35. F. X. Sureda, C. Gabriel, M. Pallas, J. Adan, J. M. Martinez, E. Escubedo, J. Camarasa, A. Camins, In vitro and in vivo protective effect of orphenadrine on glutamate neurotoxicity. Neuropharmacology. 38, 671-677 (1999).
36. C. R. Chang, C. Blackstone, Cyclic AMP-dependent protein kinase phosphorylation of Drp1 regulates its GTPase activity and mitochondrial morphology. J Biol Chem. 282, 21583-21587 (2007).
37. L. Madisen, T. A. Zwingman, S. M. Sunkin, S. W. Oh, H. A. Zariwala, H. Gu, L. L. Ng, R. D. Palmiter, M. J. Hawrylycz, A. R. Jones, E. S. Lein, H. Zeng, A robust and high-throughput Cre reporting and characterization system for the whole mouse brain. Nat Neurosci. 13, 133-140 (2010).
38. N. R. Sims, M. F. Anderson, Isolation of mitochondria from rat brain using Percoll density gradient centrifugation. Nat Protoc. 3, 1228-1239 (2008).
39. Y. Sato, S. Nakajima, N. Shiraga, H. Atsumi, S. Yoshida, T. Koller, G. Gerig, R. Kikinis, Three-dimensional multi-scale line filter for segmentation and visualization of curvilinear structures in medical images. Med Image Anal. 2, 143-168 (1998).
40. M. Kepiro, B. H. Varkuti, R. L. Davis, High content, phenotypic assays and screens for compounds nodulating cellular processes in primary neurons. Methods Enzymol, 610, 219-250 (2018).

Additional references that are cited in the present disclosure are as follows:
Alam, N. M., Mills, W. C. 4th, Wong, A. A., Douglas, R. M., Szeto, H. H., and Prusky, G. T. (2015). A mitochondrial therapeutic reverses visual decline in mouse models of diabetes. Dis Model Mech. 8, 701-710.
Brand, M. D., and Nicholls, D. G. (2011). Assessing mitochondrial dysfunction in cells. Biochem J. 435, 297-312.
Brideau, C., B. Gunter, B. Pikounis, and A. Liaw. Improved statistical methods for hit selection in high-throughput screening. J Biomol Screen. 8, 634-647 (2003).
Cai, Q., Tammineni, P. (2017) Mitochondrial aspects of synaptic dysfunction in Alzheimer's disease. J Alzheimers Dis. 57, 1087-1103.
Cai, Q., and Tammineni, P. (2016). Alternations in mitochondrial quality control in Alzheimer's disease. Front Cell Neurosci. 10, 24.
Calvo, S. E., Clauser, K. R., and Mootha, V. K. (2016). MitoCarta2.0: an updated inventory of mammalian mitochondrial proteins. Nucleic Acids Res. 44, D1251-1257.
Connolly, N. M. C., Theurey, P., Adam-Vizi, V., Bazan, N. G., Bernardi, P., Bolaflos, J. P., Culmsee, C., Dawson, V. L., Deshmukh, M., Duchen, M. R., et al. (2018). Guidelines on experimental methods to assess mitochondrial dysfunction in cellular models of neurodegenerative diseases. Cell Death Differ. 25, 542-572.
Daniele, J. R., Esping, D. J., Garcia, G., Parsons, L. S., Arriaga, E. A., and Dillin, A. (2017). “High-throughput characterization of region-specific mitochondrial function and morphology”. Sci Rep. 7(1):6749.
Ding, B., Yuan, L., Yu, H., Li, L., Ma, W., Bi, Y., Feng, J., and Xiao, R. (2011). Genistein and folic acid prevent oxidative injury induced by β-amyloid peptide. Basic Clin Pharmacol Toxicol. 108, 333-340.
Duchen, M. R. (2000). Mitochondria and calcium: from cell signalling to cell death. J Physiol. 529, 57-68.
Gerencser, A. A., Neilson, A., Choi, S. W., Edman, U., Yadava, N., Oh, R. J., Ferrick, D. A., Nicholls, D. G., and Brand, M. D. (2009). Quantitative microplate-based respirometry with correction for oxygen diffusion. Anal Chem. 81, 6868-6878.
Grimm, A., Schmitt, K., Lang, U. E., Mensah-Nyagan, A. G., and Eckert, A. (2014). Improvement of neuronal bioenergetics by neurosteroids: implications for age-related neurodegenerative disorders. Biochim Biophys Acta. 1842, 2427-2438.
Huang, S. G. (2002). Development of a high throughput screening assay for mitochondrial membrane potential in living cells. J Biomol Screen. 7, 383-389.
Hoyt, K. R., Sharma, T. A. and Reynolds, I. J. (1997). Trifluoperazine and dibucaine-induced inhibition of glutamate-induced mitochondrial depolarization in rat cultured forebrain neurones. BR J Pharmacol. 122, 803-808.
Irwin, R. W., Yao, J., Hamilton, R. T., Cadenas, E., Brinton, R. D., and Nilsen, J. (2008). Progesterone and estrogen regulate oxidative metabolism in brain mitochondria. Endocrinology. 149, 3167-3175.
Kitami, T., Logan, D. J., Negri, J., Hasaka, T., Tolliday, N.J., Carpenter, A. E., Spiegelman, B. M., Mootha, V. K. (2012). A chemical screen probing the relationship between mitochondrial content and cell size. PLoS One. 7(3):e33755.
Lezi, E., and Swerdlow, R. H. (2012). Mitochondria in neurodegeneration. Adv Exp Med Biol. 942, 269-286.
Malo, N., J. A. Hanley, S. Cerquozzi, J. Pelletier, and R. Nadon. Statistical practice in high-throughput screening data analysis. Nat Biotechnol. 24, 167-175 (2006).
Medina, J. M., Lopez-Mediavilla, C., and Orfao, A. (2002). Flow cytometry of isolated mitochondria during development and under some pathological conditions. FEBS Lett. 510, 127-132.
Moreira, P. I., Zhu, X., Wang, X., Lee, H. G., Nunomura, A., Petersen, R. B., Perry, G., and Smith, M. A. (2010). Mitochondria: A therapeutic target in neurodegeneration. Biochim Biophys Acta. 1802, 212-220.
Mortibouys, H., Aasly, J., and Bandmann, O. (2013). Ursocholanic acid rescues mitochondrial function in common forms of familial Parkinson's disease. Brain. 136, 3038-3050.
Nadanaciva, S., Murray, J., Wilson, C., Gebhard, D. F., and Will, Y. (2011). High-throughput assays for assessing mitochondrial dysfunction caused by compounds that impair mtDNA-encoded protein levels in eukaryotic cells. Curr Protoc Toxicol. Chapter 3, Unit 3. 11.
Nicholls, D. G. (2004). Mitochondrial membrane potential and aging. Aging Cell. 3, 35-40.
Nilsen, J., Irwin, R. W., Gallaher, T. K., and Brinton, R. D. (2007). Estradiol in vivo regulation of brain mitochondrial proteome. J Neurosci. 27, 14069-14077.
Oddo, S., Caccamo, A., Shepherd, J. D., Murphy, M. P., Golde, T. E., Kayed, R., Metherate, R., Mattson, M. P., Akbari, Y., and LaFerla, F M. (2003). Triple-transgenic model of Alzheimer's disease with plaques and tangles: intracellular Abeta and synaptic dysfunction. Neuron 39, 409-421.
Perez Ortiz, J. M., and Swerdlow, R. H. (2019) Mitochondrial dysfunction in Alzheimer's disease: Role in pathogenesis and novel therapeutic opportunities. Br J Pharmacol. doi: 10.1111/bph.14585. [Epub ahead of print]
Perry, S. W., Norman, J. P., Barbieri, J., Brown, E. B., and Gelbard, H. A. (2011). Mitochondrial membrane potential probes and the proton gradient: a practical usage guide. Biotechniques. 50, 98-115.
Rogers, G. W., Brand, M. D., Petrosyan, S., Ashok, D., Elorza, A. A., Ferrick, D. A., Murphy, A. N. (2011). High throughput microplate respiratory measurements using minimal quantities of isolated mitochondria. PloS One. 6 (7):e21746.
Salabei, J. K., Gibb, A. A., and Hill, B. G. (2014). Comprehensive measurement of respiratory activity in permeabilized cells using extracellular flux analysis. Nat Protoc. 9, 421-438.
Sims, N. R., and Anderson, M. F. (2008). Isolation of mitochondria from rat brain using Percoll density gradient centrifugation. Nat Protoc. 3, 1228-1239.
Sherman, S. P., and Bang, A. G. (2018). High-throughput screen for compounds that modulate neurite growth of human induced pluripotent stem cell-derived neurons. Dis Model Mech. 11(2).
Tsvetkov, A. S., Miller, J., Arrasate, M., Wong, J. S., Pleiss, M. A., and Finkbeiner, S. (2010). A small-molecule scaffold induces autophagy in primary neurons and protects against toxicity in a Huntington disease model. Proc Natl Acad Sci USA. 107, 16982-16987.
Vafai, S. B., and V. K. Mootha. (2012). Mitochondrial disorders as windows into an ancient organelle. Nature. 491, 374-383.
Voccoli, V., and Colombaioni, L. (2009). Mitochondrial remodeling in differentiating neuroblasts. Brain Res. 1252, 15-29.
Varkuti, B. H., Kepiro, M, Liu, Z., Vick, K., Avchalumov, Y., Pacifico, R., McMullen, C. M., Kamenecka, T. M., Puthanveettil, S. V., and Davis, R. L. (2020). Neuron-based high-content assay and screen for CNS active mitotherapeutics. Science Advances 6, eaaw8702.
Vos, M., Lauwers, E., and Verstreken, P. (2010). Synaptic mitochondria in synaptic transmission and organization of vesicle pools in health and disease. Front Synaptic Neurosci. 2, 139.
Wagner, B. K., Kitami, T., Gilbert, T. J., Peck, D., Ramanathan, A., Schreiber, S. L., Golub, T. R., and Mootha, V. K. (2008). Large-scale chemical dissection of mitochondrial function. Nat Biotechnol. 26, 343-351.
Wang, X., Su, B., Lee, H., Li, X., Perry G., Smith, M. A., and Zhu, X. (2009). Impaired balance of mitochondria fission and fusion in Alzheimer disease. J Neurosci. 29, 9090-9103.
Zorov, D. B., Juhaszova, M., and Sollott, S. J. (2014). Mitochondrial reactive oxygen species (ROS) and ROS-induced ROS release. Physiol Rev. 94, 909-950.
Zorova, L. D. Popkov, V. A., Plotnikov, E. Y., Silachev, D. N., Pevzner, I B, Jankauskas, S. S., Babenko, V. A., Zorov, S. D., Balakireva, A. V., Juhaszova, M., et al. (2018). Mitochondrial membrane potential. Anal Biochem. 552, 50-59.

1. An in vitro method for determining whether a test agent could be useful as a mitotherapeutic in the treatment of a patient suffering from a neurological or psychiatric disorder, comprising:
(a) contacting a test population of brain cells with a mitochondrial reporter for a time sufficient to label mitochondria in live neurons;
(b) incubating the cells with the test agent;
(c) imaging the cells to obtain a visual image of labeled mitochondria;
(d) determining mitochondrial parameters by inspection of the visual image, in comparison to an image of a control population of brain cells not incubated with the test agent, wherein the mitochondrial parameters are selected from:
concentration of cellular mitochondria;
mitochondrial length; and
mitochondrial circularity; and
(e) correlating the presence of one or more results with a conclusion that the test agent is useful as a mitotherapeutic, wherein the results are selected from:
an increase in concentration of cellular mitochondria;
increase in mitochondrial length; and
decrease in mitochondrial circularity.

2. The method according to claim 1, wherein the brain cells are from the forebrain.

3. The method according to claim 1, wherein at least two of the results are present.

4. The method according to claim 1, wherein all of the results are present.

5. An in vitro method for determining whether a test agent is likely toxic to cellular mitochondria, comprising:
(a) contacting a test population of cells with a mitochondrial reporter for a time sufficient to label mitochondria in live cells;
(b) incubating the cells with the test agent;
(c) imaging the cells to obtain a visual image of labeled mitochondria;
(d) determining mitochondrial parameters by inspection of the visual image, in comparison to an image of a control population of cells not incubated with the test agent, wherein the mitochondrial parameters are selected from:
concentration of mitochondria;
mitochondrial length; and
mitochondrial circularity; and
(e) correlating the presence of one or more results with a conclusion that the test agent is likely toxic to cellular mitochondria, wherein the results are selected from:
a decrease in concentration of cellular mitochondria;
decrease in mitochondrial length; and
increase in mitochondrial circularity.

6. The method according to claim 5, wherein the cellular mitochondria are neuronal mitochondria, and the cells are brain cells.

7. The method according to claim 1, wherein the concentration of mitochondria is the concentration of dendritic mitochondria.

8. The method according to claim 1, wherein the mitochondrial length is dendritic mitochondrial length.

9. The method according to claim 1, wherein the mitochondrial circularity is axonal mitochondrial circularity.

10. The method according to claim 1, wherein the concentration of mitochondria is the concentration of dendritic mitochondria, the mitochondrial length is dendritic mitochondrial length, and the mitochondrial circularity is axonal mitochondrial circularity.

11. An in vitro method for determining whether a test agent modulates ATP generation from cellular mitochondria, comprising:
(a) contacting a test population of cells with a mitochondrial reporter for a time sufficient to label mitochondria in live cells;
(b) incubating the test population of cells with the test agent;
(c) measuring a reporter signal from labeled mitochondria in the test population of cells; and
(d) correlating an increase, no change, or decrease in reporter signal from (c), relative to a reporter signal from a control population of cells not incubated with the test agent, to a determination that the test agent enhances, exerts no effect upon, or impairs, respectively, ATP generation from mitochondria in the test population of cells.

12. A method for treating a patient suffering from a disorder characterized by dysfunction of neuronal mitostasis or dysfunction of ATP generation, comprising administering to the patient a therapeutically effective amount of a compound, or a pharmaceutically acceptable salt thereof, selected from the following table:
cyclizine
alverine citrate
nifedipine
haloperidol
apomorphine hydrochloride
dextromethorphan hydrobromide
orphenadrine citrate
canagliflozin
pargyline hydrochloride
dipyridamole
dyclonine hydrochloride
domperidone
pyrilamine maleate
nefopam
capecitabine
yohimbine hydrochloride
xylazine
budesonide
isotretinon
esomeprazole potassium
tolnaftate
probucol
memantine hydrochloride
lamotrigine
halothane
solifenacin succinate
pyronaridine tetraphosphate
oxelaidin citrate
cloperastine hydrochloride
triclabendazole
clemizole hydrochloride
carbaril
pridinol methanesulfonate
hydroquinidine
pimethixene maleate
genistein
drofenine hydrochloride
clorgiline hydrochloride
exalamide
sulbentine
naftopidil
cholest-5-en-3-one
resveratrol 4′-methyl ether
1r,2s-phenylpropylamine
7-hydroxy-2′-methoxyisoflavone
catechin tetramethylether
2′,4-dihydroxychalcone
2′,4′-dihydroxychalcone
avocatin a
daidzein
10-hydroxycamptothecin
3,4′-dihydroxyflavone
harmine
6-hydroxyflavone
3,5-dihydroxyflavone
1,3-dideacetyl-7-deacetoxy-7-oxokhivorin
phloretin
aleuretic acid
2′,4′-dihydroxychalcone 4′-glucoside
3,7-dihydroxyflavone
dihydrofissinolide
s-isocorydine (+)
levomilnacipran hydrochloride
isocotoin
rhamnetin
euparin
4′-hydroxychalcone

13. A method for treating a patient suffering from a disorder characterized by dysfunction of neuronal mitostasis or dysfunction of ATP generation, comprising administering to the patient a therapeutically effective amount of a compound, or a pharmaceutically acceptable salt thereof, selected from the following table:
azelastine hydrochloride
pizotyline malate
doxepin hydrochloride
trimipramine maleate
orphenadrine citrate
nortriptyline hydrochloride
cyclizine
ketotifen fumarate
chlorprothixene hydrochloride
pimethixene maleate
dimenhydrinate
cyclobenzaprine hydrochloride
clemizole hydrochloride
trimeprazine tartrate
promazine hydrochloride
clozapine
thonzylamine hydrochloride
chloropyramine hydrochloride
trihexyphenidyl hydrochloride
procyclidine hydrochloride
pridinol methanesulfonate
drofenine hydrochloride
clidinium bromide
piperidolate hydrochloride
pipenzolate bromide
adiphenine hydrochloride
dyclonine hydrochloride
dibucaine hydrochloride
benoxinate hydrochloride
hycanthone
tolperisone hydrochloride
proparacaine hydrochloride
5alpha-cholestan-3beta-ol-6-one
cyclopamine
androsterone
5,4′-dimethoxy-7-hydroxyisoflavone
phenyl aminosalicylate
genistein
apigenin dimethyl ether
ketanserin tartrate
ritanserin
domperidone
pimozide
carvedilol phosphate
naftopidil
carvedilol
acetophenazine maleate
piperacetazine
thiothixene
dihydrofissinolide
1,7-dideacetoxy-1,7-dioxo-3-deacetylkhivorin
carapin-8(9)-ene
yohimbine hydrochloride
rauwolscine hydrochloride
reserpine
dexpropranolol hydrochloride [R(+)]
propranolol hydrochloride (+/−)
lidocaine hydrochloride
bupivacaine hydrochloride
toremifene citrate
clomiphene citrate
estradiol methyl ether
estrone acetate
celecoxib
diperodon hydrochloride
vinpocetine
nefazodone hydrochloride
butacaine sulfate
nebivolol hydrochloride
lobeline hydrochloride
indole-3-carbinol
trimebutine maleate
tepoxalin
meprylcaine hydrochloride
nimodipine
penfluridol
bisphenol a
nafronyl oxalate
doxazosin mesylate
benzonatate
tigecycline
ajmaline
pyrimethamine
exalamide
hydroxyzine pamoate
mefloquine
tiletamine hydrochloride
ambroxol hydrochloride
colistin sulfate
heteropeucenin, methyl ether
propafenone hydrochloride
quinine ethyl carbonate
aripiprazole
fulvestrant
bussein
canagliflozin
alverine citrate
doxorubicin
sclareol
imidazol-4-ylacetic acid sodium salt
oxiconazole nitrate
naftifine hydrochloride
sertraline hydrochloride
1-hydroxy-3,6,7-trimethoxy-2,8-diprenylxanthone
dehydroabietamide
eugenol
penbutolol sulfate
paroxetine hydrochloride
butyl paraben
triclabendazole
clemastine fumarate
levomilnacipran hydrochloride
sodium nitroprusside
medroxyprogesterone acetate
estradiol cypionate
avanafil
terconazole
oxelaidin citrate
estriol benzyl ether
larixol acetate
amitriptyline hydrochloride
imipramine hydrochloride
benazepril hydrochloride
fluoxetine hydrochloride
orlistat
bifonazole
felodipine
ancitabine hydrochloride
phytol
4′-methoxychalcone
desoxycorticosterone acetate
sparteine sulfate
chlorpromazine
nicardipine hydrochloride
pramoxine hydrochloride

14. The method according to claim 12, wherein the disorder is a neurodegenerative or neuropsychiatric disorder.

15. The method according to claim 12, wherein the disorder is selected from the group consisting of Alzheimer's disease, Parkinson's disease, amyotrophic lateral sclerosis, mood disorders, and schizophrenia.

16. The method according to claim 12, wherein the disorder is Alzheimer's disease.
